SEQUENCE LISTING (2 sequences)

SEQ ID NO: 1 - protein, 15 aa, Human
MKKHHHHHHLVPRGS

SEQ ID NO: 2 - protein, 25 aa, Artificial Sequence
  Biotinylated peptide
CPSSHSSLTERHKILHRLLQEGSPS

(19) United States
(12) Patent Application Publication
(10) Pub. No.: US 2014/0155381 A1
(43) Pub. Date: Jun. 5, 2014

(54) COMPOUNDS AND METHODS

(71) Applicants: Erkan Baloglu, Stoneham, MA (US); Gary J. Bohnert, Cambridge, MA (US); Shomir Ghosh, Cambridge, MA (US); Mercedes Lobera, Cambridge, MA (US); Darby R. Schmidt, Arlington, MA (US); Leonard Sung, Cambridge, MA (US)

(72) Inventors: Erkan Baloglu, Stoneham, MA (US); Gary J. Bohnert, Cambridge, MA (US); Shomir Ghosh, Cambridge, MA (US); Mercedes Lobera, Cambridge, MA (US); Darby R. Schmidt, Arlington, MA (US); Leonard Sung, Cambridge, MA (US)

(21) Appl. No.: 14/235,512

(22) PCT Filed: Jul. 27, 2012
(86) PCT No.: PCT/US2012/048633

§ 371 Date: Jan. 28, 2014

Related U.S. Application Data
(60) Provisional application No. 61/533,939, filed on Sep. 13, 2011.
(60) Provisional application No. 61/513,155, filed on Jul. 29, 2011.

(51) Int. Cl. C07D 413/14; C07D 407/06; C07D 407/14; C07D 413/06

(52) U.S. Cl. CPC C07D 413/14; C07D 413/06; C07D 407/06; C07D 407/14
USPC 514/211.09; 548/247; 514/378; 546/272.1; 514/337; 544/333; 514/256; 546/144; 514/307; 546/284.1; 546/256; 514/333; 540/552; 540/569; 514/221

(57) ABSTRACT

The present invention relates to novel retinoid-related orphan receptor gamma (RORγ) modulators and their use in the treatment of diseases mediated by RORγ.

The present invention relates to novel retinoid-related orphan receptor gamma (RORγ) modulators and their use in the treatment of diseases mediated by RORγ.

### BACKGROUND OF THE INVENTION

Retinoid-related orphan receptors (RORs) are transcription factors which belong to the steroid hormone nuclear receptor superfamily (Jetten & Joo (2006) Adv. Dev. Biol. 16:313-355). The ROR family consists of three members, ROR alpha (RORα), ROR beta (RORβ), and ROR gamma (RORγ), each encoded by a separate gene (RORA, RORB, and RORC, respectively). RORs contain four principal domains shared by the majority of nuclear receptors: an N-terminal A/B domain, a DNA-binding domain, a hinge domain, and a ligand binding domain. Each ROR gene generates several isoforms which differ only in their N-terminal A/B domain. Two isoforms of RORγ have been identified: RORγ1 and RORγt (also known as RORγ2). RORγ is a term used to describe both RORγ1 and/or RORγt.

While RORγ1 is expressed in a variety of tissues including thymus, muscle, kidney and liver, RORγt is exclusively expressed in the cells of the immune system. RORγt has been identified as a key regulator of Th17 cell differentiation. Th17 cells are a subset of T helper cells which produce IL-17 and other proinflammatory cytokines. Th17 cells have been shown to have key functions in several mouse autoimmune disease models including experimental autoimmune encephalomyelitis (EAE) and collagen-induced arthritis (CIA). In addition, Th17 cells or their products have been shown to be associated with the pathology of a variety of human inflammatory and autoimmune disorders including multiple sclerosis, rheumatoid arthritis, psoriasis, Crohn's disease and asthma (Jetten (2009) Nucl. Recept. Signal. 7:e003; Manel et al. (2008) Nat. Immunol. 9:641-649). The pathogenesis of chronic autoimmune diseases including multiple sclerosis and rheumatoid arthritis arises from the break in tolerance towards self-antigens and the development of auto-aggressive effector T cells infiltrating the target tissues. Studies have shown that Th17 cells are one of the important drivers of the inflammatory process in tissue-specific autoimmunity (Steinman (2008) J. Exp. Med. 205:1517-1522; Leung et al. (2010) Cell. Mol. Immunol. 7:182-189). There is evidence that Th17 cells are activated during the disease process and are responsible for recruiting other inflammatory cells types, especially neutrophils, to mediate pathology in the target tissues (Korn et al. (2009) Annu. Rev. Immunol. 27:485-517).

RORγt plays a critical role in the pathogenic responses of Th17 cells (Ivanov et al. (2006) Cell 126:1121-1133). RORγt deficient mice produce few Th17 cells. In addition, RORγt deficiency resulted in amelioration of EAE. Further support for the role of RORγt in the pathogenesis of autoimmune or inflammatory diseases can be found in the following references: Jetten & Joo (2006) Adv. Dev. Biol. 16:313-355; Meier et al. (2007) Immunity 26:643-654; Aloisi & Pujol-Borrell (2006) Nat. Rev. Immunol. 6:205-217; Jager et al. (2009) J. Immunol. 183:7169-7177; Serafini et al. (2004) Brain Pathol. 14:164-174; Magliozzi et al. (2007) Brain 130:1089-1104; Barnes (2008) Nat. Rev. Immunol. 8:183-192.

In light of the role RORγ plays in the pathogenesis of diseases, it is desirable to prepare compounds that modulate RORγ activity, which can be used in the treatment of diseases mediated by RORγ.

### SUMMARY OF THE INVENTION

The invention is directed to novel RORγ modulators and their use in the treatment of diseases mediated by RORγ. Specifically, the invention is directed to a compound according to Formula (I):

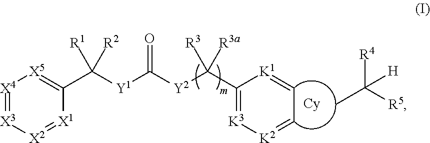

wherein: m is 0, 1, or 2;

X¹, X², X³, X⁴, and X⁵ are each independently selected from N, N⁺—O⁻, CH, and CR⁶, wherein 0-3 of X¹, X², X³, X⁴, and X⁵ are N or N⁺—O⁻ and 0-3 of X¹, X², X³, X⁴, and X⁵ are CR⁶;

one of Y¹ and Y² is O or NR⁸ and the other is a bond;

or X¹ is CR⁶, Y¹ is NR⁸, Y² is a bond, and R⁶ and R⁸ taken together with the atoms to which they are attached form a five to seven membered ring, optionally containing an additional heteroatom selected from oxygen, nitrogen, and sulfur, which ring is optionally substituted by (C₁-C₄)alkyl;

K¹, K², and K³ are each independently selected from N, CH, and CR⁶, wherein 0-2 of K¹, K², and K³ are N and 0-2 of K¹, K², and K³ are CR⁶;

R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;

R² is hydrogen, (C₁-C₆)alkyl, or (C₁-C₆)haloalkyl;

or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;

R³ and R^{3a} are each independently hydrogen, hydroxyl, (C₁-C₆)alkyl, (C₁-C₆)haloalkyl, halogen, (C₁-C₆)alkoxy, amino, (C₁-C₄)alkylamino, or ((C₁-C₄)alkyl)((C₁-C₄)alkyl)amino;

R⁴ is hydroxyl or amino;

R⁵ is phenyl or 5- or 6-membered heteroaryl, wherein said phenyl or heteroaryl is optionally substituted one, two, or three times, independently, by (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)CO₂R⁷, —((C₀-C₃)alkyl)CONR⁷R⁸, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;

each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl;

or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;

R⁹ is (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl; and

Cy taken together with the two carbon atoms of the phenyl or heteroaryl group to which it is fused comprises a five or six membered ring, optionally containing one, two, or three heteroatoms independently selected from oxygen, nitrogen, and sulfur, which ring is optionally substituted one or two times, independently, by R⁹;

provided that the compound of Formula (I) is not:

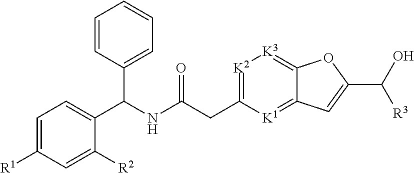

wherein:

K¹, K², and K³ are each independently selected from N and CH, wherein 0-2 of K¹, K², and K³ are N;

R¹ is F, Cl, —CH₃, or —OCH₃;

R² is —CH₃, —CN, —N(CH₃)₂, or —OCH₃; and

R³ is phenyl or 5- or 6-membered heteroaryl, wherein said phenyl or heteroaryl is optionally substituted one, two or three times, independently, by (C₁-C₄)alkyl, (C₁-C₄)haloalkyl, (C₃-C₆)cycloalkyl, halogen, cyano, hydroxyl, hydroxy(C₁-C₆)alkyl, (C₁-C₄)alkoxy, —((C₀-C₃)alkyl)CO₂(C₁-C₄)alkyl, —((C₀-C₃)alkyl)CONH₂, —((C₀-C₃)alkyl)CONH(C₁-C₄)alkyl, —((C₀-C₃)alkyl)CON((C₁-C₄)alkyl)((C₁-C₄)alkyl), or (C₁-C₄)alkoxy(C₁-C₆)alkyl;

or a salt thereof, particularly, a pharmaceutically acceptable salt thereof.

In another aspect, this invention provides a pharmaceutical composition comprising a compound of Formula (I), or a pharmaceutically acceptable salt thereof, and a pharmaceutically acceptable excipient.

In another aspect, this invention provides for the use of a compound of Formula (I) or a pharmaceutically acceptable salt thereof for the treatment of diseases mediated by RORγ. The invention further provides for the use of a compound of of Formula (I) or a pharmaceutically acceptable salt thereof as an active therapeutic substance in the treatment of a disease mediated by RORγ.

In another aspect, the invention provides a compound of Formula (I) or a pharmaceutically acceptable salt thereof for use in therapy.

In another aspect, the invention provides the use of a compound of Formula (I) or a pharmaceutically acceptable salt thereof in the manufacture of a medicament for the treatment of diseases mediated by RORγ.

Examples of such diseases for which Compounds of Formula (I) may be used include autoimmune or inflammatory diseases such as multiple sclerosis, rheumatoid arthritis, psoriasis, uveitis, dry eye, glomerulonephritis, Crohn's disease and asthma, especially psoriasis

In yet another aspect, the invention is directed to methods of treating such diseases for example by administering to a patient (e.g. human) in need thereof an effective amount of a compound of Formula (I) or a pharmaceutically acceptable salt thereof.

### DETAILED DESCRIPTION OF THE INVENTION

As used herein, the term “alkyl” represents a saturated, straight, or branched hydrocarbon moiety. The term “(C₁-C₆)alkyl” refers to an alkyl moiety containing from 1 to 6 carbon atoms. Exemplary alkyls include, but are not limited to methyl, ethyl, n-propyl, isopropyl, n-butyl, isobutyl, s-butyl, t-butyl, pentyl, and hexyl. C₀alkyl means that no alkyl group is present in the moiety. Thus, —((C₀)alkyl)CONH₂ is equivalent to —CONH₂.

When the term “alkyl” is used in combination with other substituent groups, such as “haloalkyl”, “hydroxyalkyl”, “alkoxyalkyl”, “arylalkyl”, or “heteroarylalkyl”, the term “alkyl” is intended to encompass a divalent straight or branched-chain hydrocarbon radical. For example, “arylalkyl” is intended to mean the radical-alkylaryl, wherein the alkyl moiety thereof is a divalent straight or branched-chain carbon radical and the aryl moiety thereof is as defined herein, and is represented by, for example, the bonding arrangement present in a benzyl group (—CH₂-phenyl); “halo(C₁-C₄)alkyl” is intended to mean a radical having one or more halogen atoms, which may be the same or different, at one or more carbon atoms of an alkyl moiety containing from 1 to 4 carbon atoms, which is a straight or branched-chain carbon radical, and is represented by, for example, a trifluoromethyl group (—CF₃).

As used herein, the term “cycloalkyl” refers to a non-aromatic, saturated, cyclic hydrocarbon ring. The term “(C₃-C₈)cycloalkyl” refers to a non-aromatic cyclic hydrocarbon ring having from three to eight ring carbon atoms. Exemplary “(C₃-C₈)cycloalkyl” groups useful in the present invention include cyclopropyl, cyclobutyl, cyclopentyl, cyclohexyl, cycloheptyl, and cyclooctyl.

“Alkoxy” means an alkyl radical containing the specified number of carbon atoms attached through an oxygen linking atom. The term “(C₁-C₄)alkoxy” refers to a straight- or branched-chain hydrocarbon radical having at least 1 and up to 4 carbon atoms attached through an oxygen linking atom. Exemplary “(C₁-C₄)alkoxy” groups useful in the present invention include, but are not limited to, methoxy, ethoxy, n-propoxy, isopropoxy, n-butoxy, s-butoxy, and t-butoxy.

“Aryl” represents a group or moiety comprising an aromatic, monovalent monocyclic or bicyclic hydrocarbon radical containing from 6 to 10 carbon ring atoms, to which may be fused one or more cycloalkyl rings.

Generally, in the compounds of this invention, aryl is phenyl.

Heterocyclic groups may be heteroaryl or heterocycloalkyl groups.

“Heteroaryl” represents a group or moiety comprising an aromatic monovalent monocyclic or bicyclic radical, containing 5 to 10 ring atoms, including 1 to 4 heteroatoms independently selected from nitrogen, oxygen and sulfur. This term also encompasses bicyclic heterocyclic-aryl compounds containing an aryl ring moiety fused to a heterocycloalkyl ring moiety, containing 5 to 10 ring atoms, including 1 to 4 heteroatoms independently selected from nitrogen, oxygen and sulfur. Illustrative examples of heteroaryls useful in the present invention include, but are not limited to, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, triazinyl, benzofuranyl, isobenzofuryl, 2,3-dihydrobenzofuryl, 1,3-benzodioxolyl, dihydrobenzodioxinyl, benzothienyl, indolizinyl, indolyl, isoindolyl, dihydroindolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, imidazopyridinyl, pyrazolopyridinyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl.

Generally, the heteroaryl groups present in the compounds of this invention are 5-membered and/or 6-membered monocyclic heteroaryl groups. Selected 5-membered heteroaryl groups contain one nitrogen, oxygen, or sulfur ring heteroatom, and optionally contain 1, 2, or 3 additional nitrogen ring atoms. Selected 6-membered heteroaryl groups contain 1, 2, or 3 nitrogen ring heteroatoms. Illustrative examples of 5- or 6-membered heteroaryl groups useful in the present invention include, but are not limited to furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl.

“Heterocycloalkyl” represents a group or moiety comprising a non-aromatic, monovalent monocyclic or bicyclic radical, which is saturated or partially unsaturated, containing 3 to 10 ring atoms, which includes 1 to 3 heteroatoms independently selected from nitrogen, oxygen and sulfur. Illustrative examples of heterocycloalkyls useful in the present invention include, but are not limited to, azetidinyl, pyrrolidinyl, pyrazolidinyl, pyrazolinyl, imidazolidinyl, imidazolinyl, oxazolinyl, thiazolinyl, tetrahydrofuranyl, dihydrofuranyl, 1,3-dioxolanyl, piperidinyl, piperazinyl, morpholinyl, thiomorpholinyl, tetrahydropyranyl, dihydropyranyl, 1,3-dioxanyl, 1,4-dioxanyl, 1,3-oxathiolanyl, 1,3-oxathianyl, 1,3-dithianyl, hexahydro-1H-1,4-diazepinyl, azabicylo[3.2.1]octyl, azabicylo[3.3.1]nonyl, azabicylo[4.3.0]nonyl, oxabicylo[2.2.1]heptyl and 1,5,9-triazacyclododecyl.

Generally, in the compounds of this invention, heterocycloalkyl groups are 5-7 membered heterocycloalkyl groups, such as pyrrolidinyl, pyrazolidinyl, pyrazolinyl, imidazolidinyl, imidazolinyl, oxazolinyl, thiazolinyl, tetrahydrofuranyl, dihydrofuranyl, 1,3-dioxolanyl, piperidinyl, piperazinyl, morpholinyl, thiomorpholinyl, tetrahydropyranyl, dihydropyranyl, and hexahydro-1H-1,4-diazepinyl.

It is to be understood that the terms heterocyclic, heteroaryl, and heterocycloalkyl are intended to encompass stable heterocyclic, heteroaryl, or heterocycloalkyl groups where a ring nitrogen heteroatom is optionally oxidized (e.g., heteroaryl groups containing an N-oxide, such as pyridinyl-N-oxide) or where a ring sulfur heteroatom is optionally oxidized (e.g., heterocycloalkyl groups containing sulfones or sulfoxide moieties, such as tetrahydrothienyl-1-oxide (a tetrahydrothienyl sulfoxide) or tetrahydrothienyl-1,1-dioxide (a tetrahydrothienyl sulfone)).

“Oxo” represents a double-bonded oxygen moiety; for example, if attached directly to a carbon atom forms a carbonyl moiety (C═O).

The terms “halogen” and “halo” represent chloro, fluoro, bromo, or iodo substituents. “Hydroxy” or “hydroxyl” is intended to mean the radical —OH.

“RORγ” refers to all isoforms encoded by the RORC gene which include RORγ1 and RORγt.

“RORγ modulator” refers to a chemical compound that inhibits, either directly or indirectly, the activity of RORγ. RORγ modulators include antagonists and inverse agonists of RORγ.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively.

As used herein, the term “compound(s) of the invention” means a compound of Formula (I) (as defined above) in any form, i.e., any salt or non-salt form (e.g., as a free acid or base form, or as a pharmaceutically acceptable salt thereof) and any physical form thereof (e.g., including non-solid forms (e.g., liquid or semi-solid forms), and solid forms (e.g., amorphous or crystalline forms, specific polymorphic forms, solvates, including hydrates (e.g., mono-, di- and hemi-hydrates)), and mixtures of various forms.

As used herein, the term “optionally substituted” indicates that a group, such as alkyl, cycloalkyl, alkoxy, heterocycloalkyl, aryl, or heteroaryl, may be unsubstituted, or the group may be substituted with one or more substituent(s) as defined. In the case where groups may be selected from a number of alternative groups the selected groups may be the same or different.

The term “independently” means that where more than one substituent is selected from a number of possible substituents, those substituents may be the same or different. The alternative definitions for the various groups and substituent groups of Formula (I) provided throughout the specification are intended to particularly describe each compound species disclosed herein, individually, as well as groups of one or more compound species. The scope of this invention includes any combination of these group and substituent group definitions.

Suitably, m is 0, 1, or 2. In another embodiment of this invention, m is 1.

Suitably, X¹, X², X³, X⁴, and X⁵ are each independently selected from N, N⁺—O⁻ (i.e. N-oxide), CH, and CR⁶, wherein 0-3 of) X¹, X², X³, X⁴, and X⁵ are N or N⁺—O⁻ and 0-3 of X¹, X², X³, X⁴, and X⁵ are CR⁶. In another embodiment of this invention, X¹, X², X³, X⁴, and X⁵ are each independently selected from N, N⁺—O⁻, CH, and CR⁶, wherein 0-2 of X¹, X², X³, X⁴, and X⁵ are N or N⁺—O⁻ and 0-3 of X¹, X², X³, X⁴, and X⁵ are CR⁶. In another embodiment of this invention, X¹ and X⁵ are each independently selected from N, N⁺—O⁻, CH, and CR⁶, and X², X³, and X⁴ are each independently selected from CH and CR⁶, wherein at least one of X¹ and X⁵ is N or N⁺—O⁻ and 0-3 of X¹, X², X³, X⁴, and X⁵ are CR⁶. In another embodiment of this invention, X¹ and X⁵ are each independently selected from N, N⁺—O⁻, and a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino (i.e. N, N⁺—O⁻, CH, and CR⁶, wherein R⁶ is halogen, cyano, (C₁-C₄)alkyl, (C₁-C₄)haloalkyl, (C₁-C₄)alkoxy, or ((C₁-C₄)alkyl)((C₁-C₄)alkyl)amino), and X², X³, and X⁴ are each independently a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino (i.e. CH or CR⁶, wherein R⁶ is halogen, cyano, (C₁-C₄)alkyl, (C₁-C₄)haloalkyl, (C₁-C₄)alkoxy, or ((C₁-C₄)alkyl)((C₁-C₄)alkyl)amino), wherein at least one of X¹ and X⁵ is N or N⁺—O⁻ and 2-4 of X¹, X², X³, X⁴, and X⁵ are a carbon atom substituted by hydrogen (i.e. CH). In another embodiment of this invention, X² is N or N⁺—O⁻, and X¹, X³, X⁴, and X⁵ are each independently a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino, wherein 2-4 of X¹, X³, X⁴, and X⁵ are a carbon atom substituted by hydrogen. In another embodiment of this invention, X¹, X², X³, X⁴, and X⁵ are each independently selected from CH and CR⁶, wherein 0-3 of X¹, X², X³, X⁴, and X⁵ are CR⁶. In another embodiment of this invention, X¹, X², X³, X⁴, and X⁵ are each independently a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino, wherein 2-5 of X¹, X², X³, X⁴, and X⁵ are a carbon atom substituted by hydrogen. In another embodiment of this invention, X¹ is a carbon atom substituted by halogen, (C₁-C₄)alkyl, (C₁-C₄)haloalkyl, cyano, (C₁-C₄)alkoxy, or ((C₁-C₄)alkyl)((C₁-C₄)alkyl)amino, and X², X³, X⁴, and X⁵ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino, wherein 2-4 of X², X³, X⁴, and X⁵ are a carbon atom substituted by hydrogen.

Suitably, one of Y¹ and Y² is O or NR⁸ and the other is a bond. In another embodiment of this invention, one of Y¹ and Y² is O, NH, or N((C₁-C₄)alkyl) and the other is a bond. In a specific embodiment of this invention, Y¹ is NH or NCH₃ and Y² is a bond. In another specific embodiment of this invention, Y¹ is NH and Y² is a bond. In another specific embodiment of this invention, Y¹ is a bond and Y² is NH.

In another embodiment of this invention, X¹ is CR⁶, Y¹ is NR⁸, Y² is a bond, and R⁶ and R⁸ taken together with the atoms to which they are attached form a five to seven membered ring, optionally containing an additional heteroatom selected from oxygen, nitrogen, and sulfur, which ring is optionally substituted by (C₁-C₄)alkyl. In another embodiment of this invention, X¹ is CR⁶, Y¹ is NR⁸, Y² is a bond, and R⁶ and R⁸ taken together represent —CH₂—, —CH₂CH₂—, or —CH₂CH₂CH₂—.

Suitably, K¹, K², and K³ are each independently selected from N, CH, and CR⁶, wherein 0-2 of K¹, K², and K³ are N and 0-2 of K¹, K², and K³ are CR⁶. In another embodiment of this invention, K¹, K², and K³ are each independently selected from CH and CR⁶, wherein 0-2 of K¹, K², and K³ are CR⁶. In another embodiment of this invention, K¹, K², and K³ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino (i.e. CH or CR⁶, wherein R⁶ is halogen, (C₁-C₄)alkyl, (C₁-C₄)haloalkyl, cyano, (C₁-C₄)alkoxy, or ((C₁-C₄)alkyl)((C₁-C₄)alkyl)amino), wherein 1-3 of K¹, K², and K³ are a carbon atom substituted by hydrogen (i.e. CH). In a specific embodiment of this invention, K¹, K², and K³ are each independently CH.

Suitably, R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶. In another embodiment of this invention, R¹ is (C₃-C₆)alkyl, (C₃-C₈)cycloalkyl, (C₁-C₆)alkoxy(C₁-C₂)alkyl, aryl, or heteroaryl, each of which is optionally substituted one, two, or three times, independently, by R⁶. In another embodiment of this invention, R¹ is (C₃-C₆)alkyl, (C₃-C₆)cycloalkyl, (C₁-C₆)alkoxy(C₁-C₂)alkyl, phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein said phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino (i.e. wherein R⁶ is halogen, (C₁-C₄)alkyl, (C₁-C₄)haloalkyl, cyano, (C₁-C₄)alkoxy, or ((C₁-C₄)alkyl)((C₁-C₄)alkyl)amino). In another embodiment of this invention, R¹ is (C₃-C₆)alkyl. In another embodiment of this invention, R¹ is (C₅-C₆)alkyl. In another embodiment of this invention, R¹ is phenyl or pyridinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino. In a specific embodiment of this invention, R¹ is phenyl or pyridinyl. In another specific embodiment of this invention, R¹ is phenyl.

Suitably, R² is hydrogen, (C₁-C₄)alkyl, or (C₁-C₄)haloalkyl. In another embodiment of this invention, R² is hydrogen or (C₁-C₄)alkyl. In another embodiment of this invention, R² is hydrogen or methyl. In a specific embodiment of this invention, R² is hydrogen.

In another embodiment of this invention, R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶. In another embodiment of this invention, R¹ and R² taken together represent —CH₂CH₂CH₂—, —CH₂CH₂CH₂CH₂—, or —CH₂CH₂CH₂CH₂CH₂—.

Suitably, R³ and R^{3a} are each independently hydrogen, hydroxyl, (C₁-C₆)alkyl, (C₁-C₆)haloalkyl, halogen, (C₁-C₆)alkoxy, amino, (C₁-C₄)alkylamino, or ((C₁-C₄)alkyl)((C₁-C₄)alkyl)amino. In another embodiment of this invention, R³ and R^{3a} are each independently hydrogen or methyl. In a specific embodiment of this invention, R³ and R^{3a} are each independently hydrogen.

Suitably, R⁴ is hydroxyl or amino. In a specific embodiment of this invention, R⁴ is hydroxyl. In another specific embodiment of this invention, R⁴ is amino.

Suitably, R⁵ is phenyl or 5- or 6-membered heteroaryl, wherein said phenyl or heteroaryl is optionally substituted one, two, or three times, independently, by (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)CO₂R⁷, —((C₀-C₃)alkyl)CONR⁷R⁸, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl. In another embodiment of this invention, R⁵ is 5- or 6-membered heteroaryl which is optionally substituted one, two, or three times, independently, by (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)CO₂R⁷, —((C₀-C₃)alkyl)CONR⁷R⁸, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl. In another embodiment of this invention, R⁵ is 5- or 6-membered heteroaryl which is optionally substituted one, two, or three times, independently, by (C₁-C₄)alkyl, (C₁-C₄)haloalkyl, (C₃-C₆)cycloalkyl, halogen, cyano, hydroxyl, hydroxy(C₁-C₆)alkyl, (C₁-C₄)alkoxy, —((C₀-C₃)alkyl)CO₂H, —((C₀-C₃)alkyl)CO₂(C₁-C₄)alkyl, —((C₀-C₃)alkyl)CONH₂, —((C₀-C₃)alkyl)CONH(C₁-C₄)alkyl, —((C₀-C₃)alkyl)CON((C₁-C₄)alkyl)((C₁-C₄)alkyl), (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, or ((C₁-C₄)alkyl)((C₁-C₄)alkyl)amino. In another embodiment of this invention, R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridinyl N-oxide, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino. In another embodiment of this invention, R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, each of which is optionally substituted one or two times, independently, by (C₁-C₄)alkyl. In another embodiment of this invention, R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, or isothiazolyl, each of which is optionally substituted one or two times, independently, by (C₁-C₄)alkyl. In another embodiment of this invention, R⁵ is isoxazolyl which is optionally substituted one or two times, independently, by (C₁-C₄)alkyl. In another embodiment of this invention, R⁵ is pyridinyl, pyridazinyl, pyrazinyl, pyrimidinyl, or triazinyl, each of which is optionally substituted one or two times, independently, by (C₁-C₄)alkyl. In another embodiment of this invention, R⁵ is pyridinyl which is optionally substituted one or two times, independently, by (C₁-C₄)alkyl.

Suitably, Cy taken together with the two carbon atoms of the phenyl or heteroaryl group to which it is fused comprises a five or six membered ring, optionally containing one, two, or three heteroatoms independently selected from oxygen, nitrogen, and sulfur, which ring is optionally substituted one or two times, independently, by R⁹. In another embodiment of this invention, Cy taken together with the two carbon atoms of the phenyl or heteroaryl group to which it is fused comprises a five membered aromatic ring, containing a heteroatom selected from oxygen, nitrogen, and sulfur and optionally containing an additional nitrogen atom, which ring is optionally substituted by (C₁-C₄)alkyl. In another embodiment of this invention, Cy taken together with the two carbon atoms of the phenyl or heteroaryl group to which it is fused comprises a six membered aromatic ring, optionally containing one or two nitrogen atoms, which ring is optionally substituted by (C₁-C₄)alkyl, (C₁-C₄)haloalkyl, (C₁-C₄)alkoxy, halogen, or hydroxyl.

One particular embodiment of the invention is a compound of Formula (Ia):

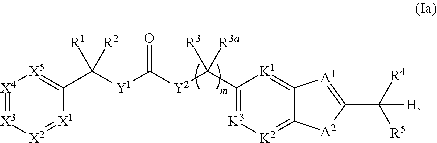

wherein:

m is 1;

X¹, X², X³, X⁴, and X⁵ are each independently selected from N, N⁺—O⁻, CH, and CR⁶, wherein 0-2 of X¹, X², X³, X⁴, and X⁵ are N or N⁺—O⁻ and 0-3 of X¹, X², X³, X⁴, and X⁵ are CR⁶;

Y¹ is NH or NCH₃ and Y² is a bond;

K¹, K², and K³ are each independently selected from N, CH, and CR⁶, wherein 0-1 of K¹, K², and K³ are N and 0-1 of K¹, K², and K³ are CR⁶;

A¹ is N, CH, or CR⁹;

A² is O, S, NH, NR⁷, NC(O)R⁷, NCO₂R⁷, or NC(O)NR⁷R⁸;

R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;

R² is hydrogen, (C₁-C₆)alkyl, or (C₁-C₆)haloalkyl;

or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;

R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;

R⁴ is hydroxyl or amino;

R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;

each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl;

or R⁷ and R⁸ taken together with the nitrogen atom to which they are attached form a four to eight membered ring, optionally containing an additional heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂H, —CO₂(C₁-C₄)alkyl, CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷; and

R⁹ is (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;

or a pharmaceutically acceptable salt thereof.

Another particular embodiment of the invention is a compound of Formula (Ia) wherein:

m is 1;

X¹ is a carbon atom substituted by halogen, (C₁-C₄)alkyl, (C₁-C₄)haloalkyl, cyano, (C₁-C₄)alkoxy, or ((C₁-C₄)alkyl)((C₁-C₄)alkyl)amino, and X², X³, X⁴, and X⁵ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino, wherein 2-4 of X², X³, X⁴, and X⁵ are a carbon atom substituted by hydrogen;

Y¹ is NH and Y² is a bond;

K¹, K², and K³ are each independently CH;

A¹ is N or CH;

A² is O, S, NH, or N((C₁-C₄)alkyl);

R¹ is phenyl optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;

R² is hydrogen;

R³ and R^{3a} are each independently hydrogen or methyl;

R⁴ is hydroxyl;

R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, each of which is optionally substituted one or two times, independently, by (C₁-C₄)alkyl;

or a pharmaceutically acceptable salt thereof.

Another particular embodiment of the invention is a compound of Formula (Ib):

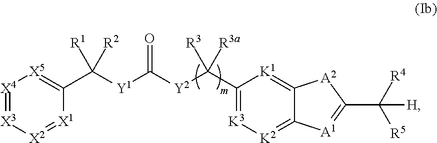

wherein:

m is 1;

X¹, X², X³, X⁴, and X⁵ are each independently selected from N, N⁺—O⁻, CH, and CR⁶, wherein 0-2 of X¹, X², X³, X⁴, and X⁵ are N or N⁺—O⁻ and 0-3 of X¹, X², X³, X⁴, and X⁵ are CR⁶;

Y¹ is NH or NCH₃ and Y² is a bond;

K¹, K², and K³ are each independently selected from N, CH, and CR⁶, wherein 0-1 of K¹, K², and K³ are N and 0-1 of K¹, K², and K³ are CR⁶;

A¹ is N, CH, or CR⁹;

A² is O, S, NH, NR⁷, NC(O)R⁷, NCO₂R⁷, or NC(O)NR⁷R⁸;

R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;

R² is hydrogen, (C₁-C₆)alkyl, or (C₁-C₆)haloalkyl;

or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;

R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;

R⁴ is hydroxyl or amino;

R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;

each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl;

or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino; and

R⁹ is (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;

or a pharmaceutically acceptable salt thereof.

Another particular embodiment of the invention is a compound of Formula (Ib) wherein:

m is 1;

X¹ is a carbon atom substituted by halogen, (C₁-C₄)alkyl, (C₁-C₄)haloalkyl, cyano, (C₁-C₄)alkoxy, or ((C₁-C₄)alkyl)((C₁-C₄)alkyl)amino, and X², X³, X⁴, and X⁵ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino, wherein 2-4 of X², X³, X⁴, and X⁵ are a carbon atom substituted by hydrogen;

Y¹ is NH and Y² is a bond;

K¹, K², and K³ are each independently CH;

A¹ is N or CH;

A² is O, S, NH, or N((C₁-C₄)alkyl);

R¹ is phenyl optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;

R² is hydrogen;

R³ and R^{3a} are each independently hydrogen or methyl;

R⁴ is hydroxyl;

R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, each of which is optionally substituted one or two times, independently, by (C₁-C₄)alkyl;

or a pharmaceutically acceptable salt thereof.

Another particular embodiment of the invention is a compound of Formula (Ic):

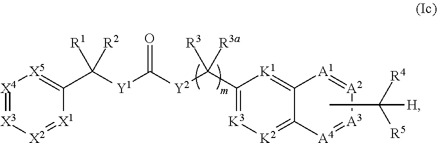

wherein:

m is 1;

X¹, X², X³, X⁴, and X⁵ are each independently selected from N, N⁺—O⁻, CH, and CR⁶, wherein 0-2 of X¹, X², X³, X⁴, and X⁵ are N or N⁺—O⁻ and 0-3 of X¹, X², X³, X⁴, and X⁵ are CR⁶;

Y¹ is NH or NCH₃ and Y² is a bond;

K¹, K², and K³ are each independently selected from N, CH, and CR⁶, wherein 0-1 of K¹, K², and K³ are N and 0-1 of K¹, K², and K³ are CR⁶;

A¹, A², A³, and A⁴ are each independently selected from N, C, CH, and CR⁹, wherein 0-2 of A¹, A², A³, and A⁴ are N, 0-1 of A¹, A², A³, and A⁴ are CR⁹, and 1 of A¹, A², A³, and A⁴ is C to which CHR⁴R⁵ is attached;

R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;

R² is hydrogen, (C₁-C₆)alkyl, or (C₁-C₆)haloalkyl;

or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;

R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;

R⁴ is hydroxyl or amino;

R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or (C₁-C₄)alkyl)((C₁-C₄)alkyl)amino;

each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl; and

R⁹ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;

or a pharmaceutically acceptable salt thereof.

Another particular embodiment of the invention is a compound of Formula (Id):

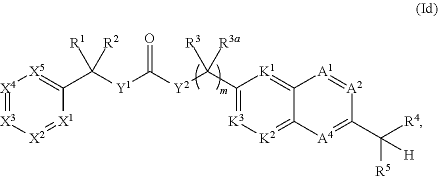

wherein:

m is 1;

X¹, X², X³, X⁴, and X⁵ are each independently selected from N, N⁺—O⁻, CH, and CR⁶, wherein 0-2 of X¹, X², X³, X⁴, and X⁵ are N or N⁺—O⁻ and 0-3 of X¹, X², X³, X⁴, and X⁵ are CR⁶;

Y¹ is NH or NCH₃ and Y² is a bond;

K¹, K², and K³ are each independently selected from N, CH, and CR⁶, wherein 0-1 of K¹, K², and K³ are N and 0-1 of K¹, K², and K³ are CR⁶;

A¹, A², and A⁴ are each independently selected from N, CH, and CR⁹, wherein 0-2 of A¹,

A², and A⁴ are N, and 0-1 of A¹, A², and A⁴ are CR⁹;

R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;

R² is hydrogen, (C₁-C₆)alkyl, or (C₁-C₆)haloalkyl;

or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;

R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;

R⁴ is hydroxyl or amino;

R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;

each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl; and

R⁹ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;

or a pharmaceutically acceptable salt thereof.

Another particular embodiment of the invention is a compound of Formula (Id) wherein:

m is 1; X¹ is a carbon atom substituted by halogen, (C₁-C₄)alkyl, (C₁-C₄)haloalkyl, cyano, (C₁-C₄)alkoxy, or ((C₁-C₄)alkyl)((C₁-C₄)alkyl)amino, and X², X³, X⁴, and X⁵ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino, wherein 2-4 of X², X³, X⁴, and X⁵ are a carbon atom substituted by hydrogen;

Y¹ is NH and Y² is a bond;

K¹, K², and K³ are each independently CH;

A¹, A², and A⁴ are each independently selected from N and CH, wherein 1-2 of A¹, A², and A⁴ are N;

R¹ is phenyl optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;

R² is hydrogen;

R³ and R^{3a} are each independently hydrogen or methyl;

R⁴ is hydroxyl; and

R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, each of which is optionally substituted one or two times, independently, by (C₁-C₄)alkyl;

or a pharmaceutically acceptable salt thereof.

Specific compounds of this invention include:
2-(2-((3,5-dimethylisoxazol-4-yl)(hydroxy)methyl)benzofuran-5-yl)-N-(1-(2-methoxy-4-methylphenyl)-4-methylpentyl)acetamide;
N-((4-chlorophenyl)(phenyl)methyl)-2-(2-((3,5-dimethylisoxazol-4-yl)(hydroxy)methyl)benzofuran-5-yl)-2-methylpropanamide;
N-((2-((3,5-dimethylisoxazol-4-yl)(hydroxy)methyl)benzofuran-5-yl)methyl)-2-(2,4-dimethylphenyl)-2-phenylacetamide;
2-(2-(amino(3,5-dimethylisoxazol-4-yl)methyl)benzofuran-5-yl)-N-((2,4-dimethylphenyl)(phenyl)methyl)acetamide;
2-(2-((3,5-dimethylisoxazol-4-yl)(hydroxy)methyl)benzo-furan-5-yl)-N-((2,6-dimethylpyridin-3-yl)(phenyl)methyl) acetamide;
2-(2-((3,5-dimethylisoxazol-4-yl)(hydroxy)methyl)benzofuran-5-yl)-N-((2,4-dimethylphenyl)(pyrimidin-2-yl)methyl)acetamide;
2-(2-((3,5-dimethylisoxazol-4-yl)(hydroxy)methyl)benzo-furan-5-yl)-N-(1-(2,4-dimethylphenyl)-4-methylpentyl)acetamide;
2-(2-((3,5-dimethylisoxazol-4-yl)(hydroxy)methyl)benzo-furan-5-yl)-N-(phenyl(p-toly)methyl)acetamide;
N-(di-o-tolylmethyl)-2-(2-((3,5-dimethylisoxazol-4-yl)(hydroxy)methyl)benzo-furan-5-yl)acetamide;
2-(2-((3,5-dimethylisoxazol-4-yl)(hydroxy)methyl)benzo-furan-5-yl)-N-(phenyl(p-toly)methyl)acetamide;
2-(2-((3,5-dimethylisoxazol-4-yl)(hydroxy)methyl)benzo-furan-5-yl)-N-((5-methylpyridin-2-yl)(phenyl)methyl)acetamide;
N-((4-chlorophenyl) (phenyl)methyl)-2-(2-((3,5-dimethylisoxazol-4-yl)(hydroxy)methyl)benzo-furan-5-yl)acetamide;
N-((2,4-dichlorophenyl) (phenyl)methyl)-2-(2-((3,5-dimethylisoxazol-4-yl)(hydroxy)methyl)benzo-furan-5-yl)acetamide;
2-(2-((3,5-dimethylisoxazol-4-yl)(hydroxy)methyl)benzo-furan-5-yl)-N-(2,4-dimethylphenyl)(pyridin-2-yl)methyl)acetamide;
2-(2-((3,5-dimethylisoxazol-4-yl)(hydroxy)methyl)benzo-furan-5-yl)-N-(3,5-dimethylpyridin-2-yl)(phenyl)methyl)acetamide;
N-(1-(2,4-dichlorophenyl)-4-methylpentyl)-2-(2-((3,5-dimethylisoxazol-4-yl)(hydroxy)methyl)benzo-furan-5-yl)acetamide;
2-(2-((3,5-dimethylisoxazol-4-yl)(hydroxy)methyl)benzofuran-5-yl)-N-(1-(2,4-dimethylphenyl)-3-isopropoxypropyl)acetamide;
2-(2-((3,5-dimethylisoxazol-4-yl)(hydroxy)methyl)benzofuran-5-yl)-N-(phenyl(1,3,5-trimethyl-1H-pyrazol-4-yl)methyl)acetamide;
N-((4-chloro-2-methylphenyl)(phenyl)methyl)-2-(2-((3,5-dimethylisoxazol-4-yl)(hydroxy)methyl)benzofuran-5-yl)acetamide;
N-((4-chlorophenyl)(phenyl)methyl)-2-(2-((3,5-dimethylisoxazol-4-yl)(hydroxy)methyl)benzofuran-5-yl)acetamide;
N-((2,4-dichlorophenyl)(phenyl)methyl)-2-(2-((3,5-dimethylisoxazol-4-yl)(hydroxy)methyl)benzofuran-5-yl)acetamide;
2-(2-((3,5-dimethylisoxazol-4-yl)(hydroxy)methyl)benzofuran-5-yl)-N-((2,4-dimethylphenyl)(pyridin-2-yl)methyl)acetamide;
N-(di-p-tolylmethyl)-2-(2-((3,5-dimethylisoxazol-4-yl)(hydroxy)methyl)benzofuran-5-yl)acetamide;
2-(2-((3,5-dimethylisoxazol-4-yl)(hydroxy)methyl)benzofuran-5-yl)-N-(1-(2,6-dimethylpyridin-3-yl)-4-methylpentyl)acetamide;
2-(2-((3,5-dimethylisoxazol-4-yl)(hydroxy)methyl)benzofuran-5-yl)-N-(4-methyl-1-(p-tolyl)pentyl)acetamide;
2-(2-((3,5-dimethylisoxazol-4-yl)(hydroxy)methyl)benzofuran-5-yl)-N-(1-(3,5-dimethylpyridin-2-yl)-4-methylpentyl)acetamide;
2-(2-((3,5-dimethylisoxazol-4-yl)(hydroxy)methyl)benzofuran-5-yl)-N-(4-methyl-1-(4-methyl-2-(trifluoromethyl)phenyl)pentyl)acetamide;
N-(1-(2,4-dichlorophenyl)-4-methylpentyl)-2-(2-((3,5-dimethylisoxazol-4-yl)(hydroxy)methyl)benzofuran-5-yl)acetamide;
N-(1-(4-chloro-2,6-difluorophenyl)-4-methylpentyl)-2-(2-((3,5-dimethylisoxazol-4-yl)(hydroxy)methyl)benzofuran-5-yl)acetamide;
N-((4-chloro-2,6-difluorophenyl)(phenyl)methyl)-2-(2-((3,5-dimethylisoxazol-4-yl)(hydroxy)methyl)benzofuran-5-yl)acetamide;
2-(2-((S)-(3,5-dimethylisoxazol-4-yl)(hydroxy)methyl)benzofuran-5-yl)-1-(6-methyl-1-phenyl-3,4-dihydroisoquinolin-2(1H)-yl)ethanone;
N-((4-chloro-2-methylphenyl)(phenyl)methyl)-2-(2-((S)-(3,5-dimethylisoxazol-4-yl)(hydroxy)methyl)benzofuran-5-yl)acetamide;
2-(2-((3,5-dimethylisoxazol-4-yl)(hydroxy)methyl)benzo-furan-5-yl)-N-((2,4-dimethylphenyl)(phenyl) methyl)-2-methylpropanamide;
2-(2-((3,5-dimethylisoxazol-4-yl)(hydroxy)methyl)benzo-furan-5-yl)-N-((2,4-dimethylphenyl)(pyridin-2-yl)methyl)-2-methylpropanamide;
2-(2-((3,5-dimethylisoxazol-4-yl)(hydroxy)methyl)benzofuran-5-yl)-N-(1-(2,4-dimethylphenyl)-2-isopropoxyethyl)acetamide;
2-(2-((3,5-dimethylisoxazol-4-yl)(hydroxy)methyl)benzofuran-5-yl)-N-((4-hydroxy-2-methylphenyl)(phenyl)methyl)acetamide;
2-(2-((3,5-dimethylisoxazol-4-yl)(hydroxy)methyl)benzofuran-5-yl)-N-((2,4-dimethylphenyl)(4-(methylsulfonamido)phenyl)methyl)acetamide;
N-((2-chloro-4-methylphenyl)(phenyl)methyl)-2-(2-(hydroxy(pyridin-4-yl)methyl)benzofuran-5-yl)acetamide;
2-(2-((3,5-dimethylisoxazol-4-yl)(hydroxy)methyl)benzofuran-5-yl)-N-(1-(2,6-dimethylpyridin-3-yl)-4-methylpentyl)acetamide;
N-(1-(2-(dimethylamino)-4-methylphenyl)-4-methylpentyl)-2-(2-((3,5-dimethylisoxazol-4-yl)(hydroxy)methyl)benzofuran-5-yl)acetamide;
2-(2-((S)-(3,5-dimethylisoxazol-4-yl)(hydroxy)methyl)benzofuran-5-yl)-N-((S)-1-(3,5-dimethylpyridin-2-yl)-4-methylpentyl)acetamide;
2-(2-((S)-(3,5-dimethylisoxazol-4-yl)(hydroxy)methyl)benzofuran-5-yl)-N-((R)-1-(3,5-dimethylpyridin-2-yl)-4-methylpentyl)acetamide;
2-(2-((R)-(3,5-dimethylisoxazol-4-yl)(hydroxy)methyl)benzofuran-5-yl)-N—((R)-1-(3,5-dimethylpyridin-2-yl)-4-methylpentyl)acetamide;
2-(2-((R)-(3,5-dimethylisoxazol-4-yl)(hydroxy)methyl)benzofuran-5-yl)-N-((S)-1-(3,5-dimethylpyridin-2-yl)-4-methylpentyl)acetamide;
2-(2-((3,5-dimethylisoxazol-4-yl)(hydroxy)methyl)benzofuran-5-yl)-N-(1-(3,5-dimethylpyridin-2-yl)-1-deutero-4-methylpentyl)acetamide;
2-(2-((3,5-dimethylisoxazol-4-yl)(hydroxy)methyl)benzofuran-5-yl)-N-((2,5-dimethyloxazol-4-yl)(phenyl)methyl)acetamide;
N-(1-(3,5-dimethylpyridin-2-yl)-4-methylpentyl)-2-(2-(hydroxy(pyridin-4-yl)methyl)benzofuran-5-yl)acetamide;
2-(2-((3,5-dimethylisoxazol-4-yl)(hydroxy)methyl)benzofuran-5-yl)-N-(1-(2,5-dimethyloxazol-4-yl)-4-methylpentyl)acetamide;
2-(4-((2-(2-(3,5-dimethylisoxazol-4-yl)(hydroxy)methyl)benzofuran-5-yl)acetamido)(phenyl)methyl)-3-methylphenoxy)acetic acid;
2-(2-((3,5-dimethylisoxazol-4-yl)(hydroxy)methyl)benzofuran-5-yl)-N-((2-hydroxy-4,6-dimethoxyphenyl)(phenyl)methyl)acetamide;
2-(2-((3,5-dimethylisoxazol-4-yl)(hydroxy)methyl)-6-fluorofuro[3,2-b]pyridin-5-yl)-N-((2,4-dimethylphenyl)(phenyl)methyl)acetamide;
2-(2-((3,5-dimethylisoxazol-4-yl)(hydroxy)methyl)benzofuran-5-yl)-1-(1-(4-fluorophenyl)-6-methyl-3,4-dihydroisoquinolin-2(1H)-yl)ethanone;
N—((S)-(2,4-dimethylphenyl)(phenyl)methyl)-2-(2-(hydroxy(pyridin-4-yl)methyl)furo[3,2-b]pyridin-5-yl)acetamide;
2-(2-((S)-(3,5-dimethylisoxazol-4-yl)(hydroxy)methyl)benzofuran-5-yl)-N-((4-isocyano-2-methylphenyl)(phenyl)methyl)acetamide;
2-(2-((S)-(3,5-dimethylisoxazol-4-yl)(hydroxy)methyl)benzofuran-5-yl)-N-((3-methylpyridin-4-yl)(phenyl)methyl)acetamide;
2-(2-((3,5-dimethylisoxazol-4-yl)(hydroxy)methyl)benzofuran-5-yl)-1-(5-phenyl-2,3-dihydrobenzo[f][1,4]oxazepin-4(5H)-yl)ethanone;
1-(5-(4-chlorophenyl)-2,3-dihydrobenzo[f][1,4]oxazepin-4(5H)-yl)-2-(2-((3,5-dimethylisoxazol-4-yl)(hydroxy)methyl)benzofuran-5-yl)ethanone;
2-(2-((3,5-dimethylisoxazol-4-yl)(hydroxy)methyl)benzofuran-5-yl)-N-((4-(hydroxymethyl)-2-methylphenyl)(phenyl)methyl)acetamide;
1-(6-chloro-1-phenyl-3,4-dihydroisoquinolin-2(1H)-yl)-2-(2-((3,5-dimethylisoxazol-4-yl)(hydroxy)methyl)benzofuran-5-yl)ethanone;
1-(8-chloro-1-phenyl-3,4-dihydroisoquinolin-2(1H)-yl)-2-(2-((3,5-dimethylisoxazol-4-yl)(hydroxy)methyl)benzofuran-5-yl)ethanone;
2-(2-((3,5-dimethylisoxazol-4-yl)(hydroxy)methyl)benzofuran-5-yl)-1-(6-methoxy-1-phenyl-3,4-dihydroisoquinolin-2(1H)-yl)ethanone;
2-(2-((3,5-dimethylisoxazol-4-yl)(hydroxy)methyl)benzofuran-5-yl)-N-((2,4-dimethylphenyl)(4-fluorophenyl)methyl)acetamide;
1-(1,8-dimethyl-5-phenyl-2,3-dihydro-1H-benzo[e][1,4]diazepin-4(5H)-yl)-2-(2-((3,5-dimethylisoxazol-4-yl)(hydroxyl)methyl)benzofuran-5-yl)ethanone;

and pharmaceutically acceptable salts thereof.

The meaning of any functional group or substituent thereon at any one occurrence in Formula (I), or any subformula thereof, is independent of its meaning, or any other functional group's or substituent's meaning, at any other occurrence, unless stated otherwise.

It is to be understood that in a compound of Formula (I) wherein m is 2, each instance of R³ is selected independently from the other R³. Similarly, in a compound of Formula (I) wherein m is 2, each instance of R^{3a} is selected independently from the other R^{3a}.

The compounds according to Formula (I) may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in Formula (I), or in any chemical structure illustrated herein, is not specified the structure is intended to encompass all individual stereoisomers and all mixtures thereof. Thus, compounds according to Formula (I) containing one or more chiral center may be used as racemic mixtures, enantiomerically enriched mixtures, or as enantiomerically pure individual stereoisomers.

Individual stereoisomers of a compound according to Formula (I) which contain one or more asymmetric centers may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

“Enantiomerically enriched” refers to products whose enantiomeric excess is greater than zero. For example, enantiomerically enriched refers to products whose enantiomeric excess is greater than 50% ee, greater than 75% ee, and greater than 90% ee.

“Enantiomeric excess” or “ee” is the excess of one enantiomer over the other expressed as a percentage. As a result, since both enantiomers are present in equal amounts in a racemic mixture, the enantiomeric excess is zero (0% ee). However, if one enantiomer was enriched such that it constitutes 95% of the product, then the enantiomeric excess would be 90% ee (the amount of the enriched enantiomer, 95%, minus the amount of the other enantiomer, 5%).

“Enantiomerically pure” means products whose enantiomeric excess is 99% ee or greater.

When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.” It is to be understood that when named or depicted by structure, the disclosed compound, or solvates (particularly, hydrates) thereof, also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

For solvates of the compounds of Formula (I), or salts thereof, that are in crystalline form, the skilled artisan will appreciate that pharmaceutically acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

Because of their potential use in medicine, the salts of the compounds of Formula (I) are preferably pharmaceutically acceptable. Suitable pharmaceutically acceptable salts include those described by Berge, Bighley and Monkhouse J. Pharm. Sci (1977) 66, pp 1-19. Salts encompassed within the term “pharmaceutically acceptable salts” refer to non-toxic salts of the compounds of Formula (I).

Salts of the compounds of Formula (I) containing a basic amine or other basic functional group may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates, γ-hydroxybutyrates, glycolates, tartrates mandelates, and sulfonates, such as xylenesulfonates, methanesulfonates, propanesulfonates, naphthalene-1-sulfonates and naphthalene-2-sulfonates.

Salts of the compounds of Formula (I) containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acid such as lysine and arginine.

Other non-pharmaceutically acceptable salts, e.g. trifluoroacetate, may be used, for example in the isolation of compounds of the invention, and are included within the scope of this invention.

The invention includes within its scope all possible stoichiometric and non-stoichiometric forms of the salts of the compounds of Formula (I).

If a compound of Formula (I) containing a basic amine or other basic functional group is isolated as a salt, the corresponding free base form of that compound may be prepared by any suitable method known to the art, including treatment of the salt with an inorganic or organic base, suitably an inorganic or organic base having a higher pKₐ than the free base form of the compound. Similarly, if a compound of Formula (I) containing a carboxylic acid or other acidic functional group is isolated as a salt, the corresponding free acid form of that compound may be prepared by any suitable method known to the art, including treatment of the salt with an inorganic or organic acid, suitably an inorganic or organic acid having a lower pKₐ than the free acid form of the compound.

The invention also includes various deuterated forms of the compounds of Formula (I). Each available hydrogen atom attached to a carbon atom may be independently replaced with a deuterium atom. A person of ordinary skill in the art will know how to synthesize deuterated forms of the compounds of Formula (I). Commercially available deuterated starting materials may be employed in the preparation of deuterated forms of the compounds of Formula (I), or they may be synthesized using conventional techniques employing deuterated reagents (e.g. lithium aluminum deuteride or sodium borodeuteride).

### Methods of Use

Modulators of RORγ can be useful in the treatment of diseases mediated by RORγ, particularly autoimmune or inflammatory diseases and cancer. Such inflammatory or autoimmune diseases include multiple sclerosis, rheumatoid arthritis, psoriasis, Crohn's disease, inflammatory bowel disease, graft-versus-host disease (GVHD), Sjorgen's syndrome, optic neuritis, chronic obstructive pulmonary disease, asthma, type I diabetes, neuromyelitis optica, myasthenia gravis, uveitis, Behcets disease, Guillain-Barre syndrome, psoriatic arthritis, Graves' disease, allergic contact dermatitis, systemic lupus erythematosus, cutaneous lupus erythematosus, ankylosing spondylitis, Hashimoto Thyroiditis, dry eye and glomerulonephritis, myocarditis, especially psoriasis Such cancers include multiple myeloma and lytic bone disease associated with multiple myeloma, acute myelogenous leukemia (AML), head and neck squamous cell carcinoma, bladder carcinoma, gastric cancer, hepatocellular carcinoma, melanoma, medulloblastoma and colon cancer. Accordingly, in another aspect the invention is directed to methods of treating such diseases using a compound of Formula (I) or a pharmaceutically acceptable salt thereof. The methods of treatment of the invention comprise administering an effective amount of a compound according to Formula (I) or a pharmaceutically acceptable salt thereof to a patient (particularly a human) in need thereof.

In a further aspect, the invention is directed to a compound of Formula (I) or a pharmaceutically acceptable salt thereof for use in therapy. In particular, for use in the treatment of diseases mediated by RORγ, particularly autoimmune or inflammatory diseases and cancer, such as those disclosed above.

In a further aspect, the invention is directed to the use of a compound of Formula (I) or a pharmaceutically acceptable salt thereof in the manufacture of a medicament for the treatment of diseases mediated by RORγ, particularly autoimmune or inflammatory diseases and cancer, such as those disclosed above.

As used herein, “treatment” in reference to a condition means: (1) the amelioration or prevention of the condition being treated or one or more of the biological manifestations of the condition being treated, (2) the interference with (a) one or more points in the biological cascade that leads to or is responsible for the condition being treated or (b) one or more of the biological manifestations of the condition being treated, or (3) the alleviation of one or more of the symptoms or effects associated with the condition being treated.

As indicated above, “treatment” of a condition includes prevention of the condition. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

An “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, “patient” refers to a human or a mammal, especially a human.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, otic, intravaginal, and intranasal administration.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the amount administered and the duration such regimens are administered, for a compound of the invention depend on the condition being treated, the severity of the condition being treated, the age and physical condition of the patient being treated, the medical history of the patient to be treated, the nature of concurrent therapy, the particular route of administration chosen, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change. Typical daily dosages range from 1 mg to 1000 mg.

It will be appreciated by those skilled in the art that certain protected derivatives of compounds of Formula (I), which may be made prior to a final deprotection stage, may not possess pharmacological activity as such, but may, in certain instances, be administered orally or parenterally and thereafter metabolized in the body to form compounds of the invention which are pharmacologically active. Such derivatives may therefore be described as “prodrugs”. Further, certain compounds of the invention may act as prodrugs of other compounds of the invention. All protected derivatives and prodrugs of compounds of the invention are included within the scope of the invention.

Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Preferred “pro-moieties” for compounds of the invention include: ester, carbonate ester, hemi-ester, phosphate ester, nitro ester, sulfate ester, sulfoxide, amide, carbamate, azo-, phosphamide, glycoside, ether, acetal, and ketal derivatives of the compounds of Formula (I).

Administration of a compound of the invention as a prodrug may enable the skilled artisan to do one or more of the following: (a) modify the onset of the compound in vivo; (b) modify the duration of action of the compound in vivo; (c) modify the transportation or distribution of the compound in vivo; (d) modify the solubility of the compound in vivo; and (e) overcome or overcome a side effect or other difficulty encountered with the compound.

The invention further includes the use of compounds of the invention as an active therapeutic substance, in particular in the treatment of diseases mediated by RORγ. In another embodiment, the invention relates to the use of compounds of the invention in the preparation of a medicament for the treatment of diseases mediated by RORγ.

Examples of such diseases include autoimmune or inflammatory diseases such as multiple sclerosis, rheumatoid arthritis, psoriasis, Crohn's disease, inflammatory bowel disease, Sjorgen's syndrome, optic neuritis, chronic obstructive pulmonary disease, asthma, type I diabetes, neuromyelitis optica, Myasthenia Gravis, uveitis, Guillain-Barre syndrome, psoriatic arthritis, Graves' disease, allergic contact dermatitis, systemic lupus erythematosus, cutaneous lupus erythematosus, ankylosing spondylitis, Hashimoto Thyroiditis, Dry Eye, glomerulonephritis, myocarditis and cancer diseases including multiple myeloma and lytic bone disease associated with multiple myeloma, acute myelogenous leukemia (AML), head and neck squamous cell carcinoma, bladder carcinoma, gastric cancer, hepatocellular carcinoma, melanoma, medulloblastoma and colon cancer.

The invention includes the use of compounds of the invention for the preparation of a composition for treating or ameliorating diseases mediated by RORγ in a subject in need thereof, wherein the composition comprises a mixture of one or more of the compounds of the invention and an optional pharmaceutically acceptable excipient.

The compounds of the invention may be used alone or in combination with one or more other therapeutic agents. Accordingly the present invention provides a combination comprising a compound of Formula (I) or a pharmaceutically acceptable salt thereof and one or more other therapeutic agents. Such combinations may be presented individually (wherein each active is in separate composition) or the actives are presented in a combined composition.

This invention provides a combination of a compound of Formula (I), or a pharmaceutically acceptable salt thereof, and one or more therapeutic agents for the treatment of an inflammatory disease and/or an autoimmune disease, for example, a TNF-a inhibitor; a non-selective COX-1/COX-2 inhibitor; a selective COX-2 inhibitor, such as celecoxib; agents including methotrexate, leflunomide, sulfasalazine, azathioprine, penicillamine, bucillamine, actarit, mizoribine, lobenzarit, hydroxychloroquine, d-penicillamine, aurothiomalate, auranofin, parenteral and/or oral gold, cyclophosphamide, a BAFF/APRIL inhibitor, CTLA-4-Ig, or a mimetic of CTLA-4-Ig; 5-lipoxygenase (5-LO) inhibitor, or a 5-lipoxygenase activating protein (FLAP) antagonist; a leukotriene modifier, including a leukotriene receptor antagonist, such as montelukast, zafirlukast, pranlukast; a phosphodiesterase type IV (PDE-IV) inhibitor, such as cilomilast (ariflo) or roflumilast; an antihistamine H1 receptor antagonist; anticholinergic agents such as muscarinic antagonists (ipratropium bromide and tiotropium bromide), as well as selective muscarinic M3 antagonists; β-adrenoceptor agonists such as salmeterol, formoterol, arformoterol, terbutaline, metaproterenol, albuterol and the like; a DP receptor antagonist, such as S-5751 and laropiprant; TP receptor antagonists such as seratrodast; neurokinin antagonists (1 NK2); VLA-4 antagonists; a corticosteroid, such as triamcinolone acetonide, budesonide, beclomethasone, fluticasone and mometasone; insulin-like growth factor type I (IGF-1) mimetic; kinase inhibitors including Janus Kinase inhibitors (e.g., JAK 1 and/or JAK2 and/or JAK 3 and/or TYK2), p38 MAPK, Syk or IKK2; rituximab; selective co-stimulation modulator such as abatacept; IL-1 inhibitor anakinra, IL-6 inhibitor tocilizumab, and IL12/IL-23 inhibitor ustekimumab; anti-IL17 antibody, anti-IL17R antibody, anti-IL21 antibody, or anti-IL22 antibody, S1P1 agonists including fingolimod; interferon beta 1; natalizumab; a mTOR inhibitor such as rapamycin, cyclosporine, tacrolimus; non-steroidal antiinflammatory agent (NSAID), including alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen, indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac, flufenamic acid, meclofenamic acid, mefenamic acid, niflumic acid, tolfenamic acid, diflunisal and flufenisal, isoxicam, piroxicam, sudoxicam, tenoxican, acetyl salicylic acid, apazone, bezpiperylon, feprazone, mofebutazone, oxyphenbutazone, phenylbutazone; fumaric acid derivative, BG-12; chemokine or chemokine receptor inhibitor, such as a CCR-1, CCR-2, CCR-3 and CCR-9 antagonist.

This invention further provides a combination of a compound of Formula (I), or a pharmaceutically acceptable salt thereof, and one or more therapeutic agents for the treatment of multiple myeloma, for example, Bortezomib-dexamethasone, Bortezomib-dexamethasone-cyclophosphamide, Bortezomib-dexamethasone-lenalidomide, Lenalidomide-dexamethasone, Melphalan-prednisone-thalidomide, Melphalan-prednisone-bortezomib, Melphalan-prednisone-lenalidomide, Lenalidomide-dexamethasone-clarithromycin and any of the above combinations plus agents used to treat bone disease in multiple myeloma including bisphosponates, RANK-L inhibitors such as Denusomab and anabolic bone building drugs such as parathyroid hormone (PTH).

This invention also provides a combination of a compound of Formula (I), or a pharmaceutically acceptable salt thereof, and one or more therapeutic agents for the treatment of colon and/or rectal cancer, for example FOLFOX® (leucovorin [folinic acid], 5-Fluoruracil, and oxaliplatin), FOLFIRI® (leucovorin, 5-Fluoruracil, and irinotecan), CapeOX® (capecitabine and oxaliplatin), any of the above combinations plus either bevacizumab or cetuximab (but not both), 5-Fluoruracil and leucovorin, with or without bevacizumab, Capecitabine, with or without bevacizumab, FOLFOXIRI® (leucovorin, 5-Fluoruracil, oxaliplatin, and irinotecan), Irinotecan, with or without cetuximab, Cetuximab alone, and Panitumumab alone.

### Compositions

The compounds of the invention will normally, but not necessarily, be formulated into pharmaceutical compositions prior to administration to a patient. Accordingly, in another aspect the invention is directed to pharmaceutical compositions comprising a compound of the invention and one or more pharmaceutically acceptable excipient(s).

The pharmaceutical compositions of the invention may be prepared and packaged in bulk form wherein an effective amount of a compound of the invention can be extracted and then given to the patient such as with powders, syrups, and solutions for injection. Alternatively, the pharmaceutical compositions of the invention may be prepared and packaged in unit dosage form. For oral application, for example, one or more tablets or capsules may be administered. A dose of the pharmaceutical composition contains at least a therapeutically effective amount of a compound of this invention (i.e., a compound of Formula I or a salt, particularly a pharmaceutically acceptable salt, thereof). When prepared in unit dosage form, the pharmaceutical compositions may contain from 1 mg to 1000 mg of a compound of this invention.

The pharmaceutical compositions of the invention typically contain one compound of the invention. However, in certain embodiments, the pharmaceutical compositions of the invention contain more than one compound of the invention. For example, in certain embodiments the pharmaceutical compositions of the invention contain two compounds of the invention. In addition, the pharmaceutical compositions of the invention may optionally further comprise one or more additional therapeutically active compounds.

As used herein, “pharmaceutically acceptable excipient” means a pharmaceutically acceptable material, composition, or vehicle involved in giving form or consistency to the pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically acceptable.

The compounds of the invention and the pharmaceutically acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as dry powders, aerosols, suspensions, and solutions; and (6) topical administration such as creams, ointments, lotions, solutions, pastes, sprays, foams, and gels.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of the compound or compounds of the invention once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

### Compound Preparation

The compounds of Formula (I) may be obtained by using synthetic procedures illustrated in the Schemes below or by drawing on the knowledge of a skilled organic chemist. The reaction sequences provided in Scheme 1 are applicable for producing compounds of the invention having a variety of different X¹-X⁵, R¹, R³, R^{3a}, and R⁵ groups, as defined herein, employing appropriate precursors. The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999). In some instances, a substituent may be specifically selected to be reactive under the reaction conditions used. Under these circumstances, the reaction conditions convert the selected substituent into another substituent that is either useful as an intermediate compound or is a desired substituent in a target compound.

The compounds of Formula (I) containing a benzofuran moiety may be prepared from commercially available phenol derivatives according to the following scheme s. Substituted aryl methyl amines of formula (II) may be prepared from commercially available aryl nitrile starting materials according to Scheme 1 and Scheme 2.

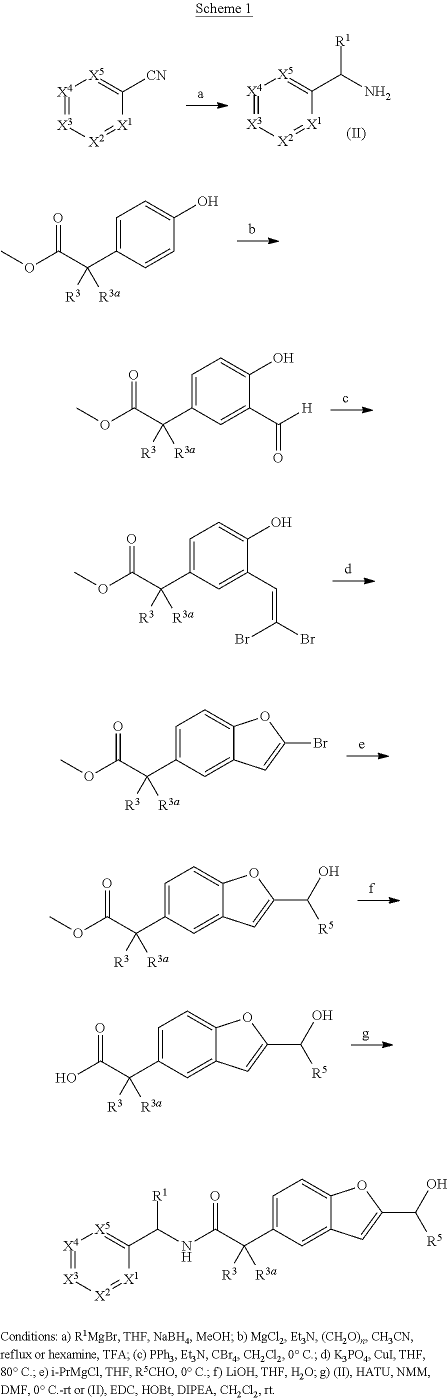

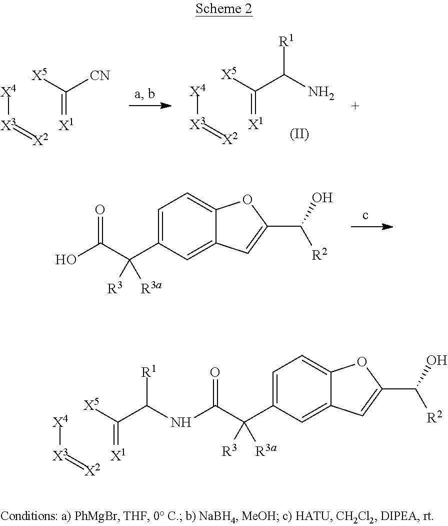

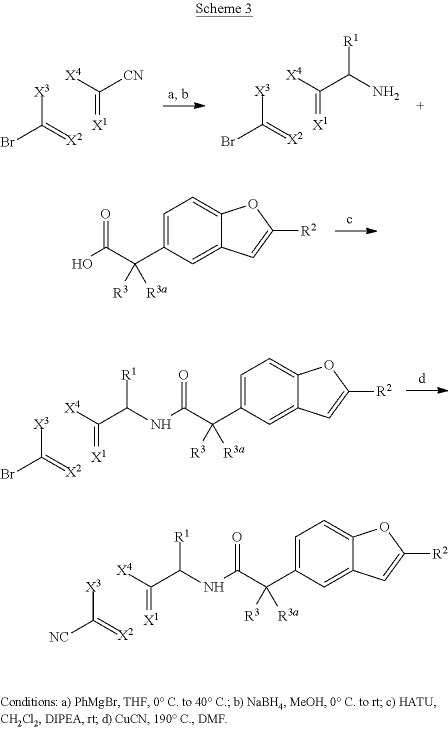

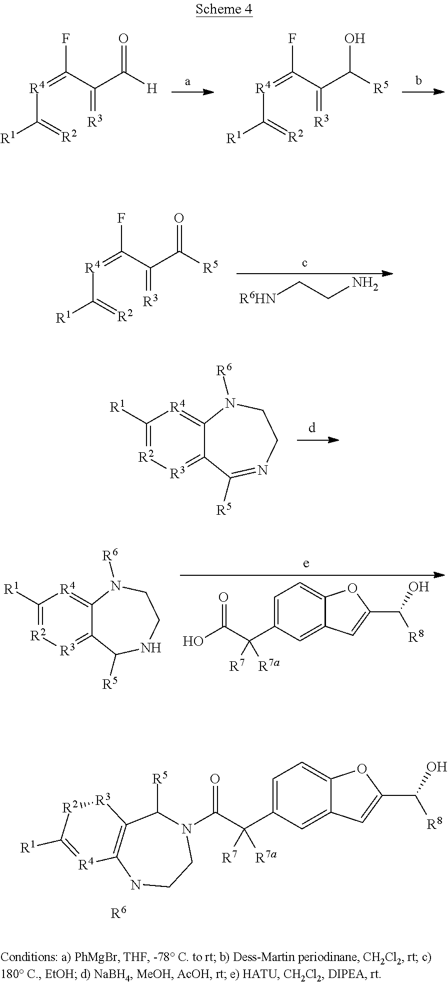

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

Compounds names were generated using the software program ChemBioDraw Ultra V 12.0 available from CambridgeSoft Corporation, 100 CambridgePark Drive, Cambridge, Mass. 02140 USA (http://www.cambridgesoft.com).

### ABBREVIATIONS

AcOH acetic acid
AlCl₃ aluminum trichloride
aq. aqueous
CBr₄ carbon tetrabromide
CH₂Cl₂ dichloromethane
CH₃CN acetonitrile
(CH₂O)ₙ paraformaldehyde
CH₃SO₃H methanesulfonic acid
conc. concentrated
CuCN copper(I) cyanide
CuI copper(I) iodide
(COCl)₂ oxalyl chloride

### DIPEA N,N-diisopropylethylamine

DMAP 4-(dimethylamino)pyridine
DME 1,2-dimethoxyethane

### DMF N,N-dimethylformamide

DMSO dimethylsulfoxide
Et₂O diethyl ether
EtOAc ethyl acetate
EDC N-(3-dimethylaminopropyl)-N′-ethylcarbodiimide hydrochloride
Et₃N triethylamine
EtOH ethanol
FeSO₄ iron (II) sulfate
h hour(s)
HATU O-(7-azabenzotriazol-1-yl)-N,N,N′,N′-tetramethyluronium hexafluorophosphate
HCl hydrochloric acid
H₂O water
HNO₃ nitric acid
HOBt hydroxybenzotriazole
H₂SO₄ sulfuric acid
i-PrMgCl isopropylmagnesium chloride
K₂CO₃ potassium carbonate
K₃Fe(CN)₆ potassium ferricyanide
K₃PO₄ potassium phosphate tribasic
LCMS liquid chromatography mass spectrometry
LiAlH₄ lithium aluminum hydride
LiOH lithium hydroxide
m-CPBA meta-chloroperbenzoic acid
MeOH methanol
MgCl₂ magnesium chloride
min minute(s)
NaBH₄ sodium borohydride
n-BuLi n-butyllithium
NaCN sodium cyanide
Na₂CO₃ sodium carbonate
NaHCO₃ sodium bicarbonate
NaHSO₃ sodium bisulfite
NaN₃ sodium azide
NaOH sodium hydroxide
Na₂SO₄ sodium sulfate
NH₄Cl ammonium chloride

### NMM N-methylmorpholine

Pd/C palladium on carbon
Pd(dppf)Cl₂ [1,1′-bis(diphenylphosphino)ferrocene]dichloropalladium(II)
Pd(PPh₃)₄ tetrakis(triphenylphosphine)palladium(0)
PhNO₂ nitrobenzene
POCl₃ phosphoryl chloride
PPh₃ triphenylphosphine
p-TsOH para-toluene sulfonic acid
rt room temperature
SnCl₄ tin (IV) chloride
SOCl₂ thionyl chloride
TFA trifluoroacetic acid
THF tetrahydrofuran
®T3P 2,4,6-tripropyl-1,3,5,2,4,6-trioxatriphosphorinane 2,4,6-trioxide
Zn zinc

### LCMS Conditions

LCMS-TFA: Column: Zorbax XDB C18, 3.5 μm, 50×4.6 mm; Temperature: 35° C.; Mobile Phase: water (0.05% TFA) B: acetonitrile (0.05% TFA); Gradient: 5% B for 0.1 min, increase to 100% B within 7 min, return to 5% B within 0.1 min, 5% B for 3 min.; Flow Rate: 1.0 mL/min; Detection: PDA 190-400 nm, (analyze at 220, 254, 280 nm)

LCMS-AMF: Column: Zorbax XDB C18, 3.5 μm, 50×4.6 mm; Temperature: 35° C.; Mobile Phase: water (10 mM ammonium formate) B: acetonitrile; Gradient: 5% B for 0.1 min, increase to 100% B within 7 min, 100%, return to 5% B within 0.1 min, 5% B for 3 min.; Flow Rate: 1.0 mL/min; Detection: PDA 190-400 nm, (analyze at 220, 254, 280 nm)

### Preparation 1

### 1-(3,5-dimethylisoxazol-4-yl)prop-2-yn-1-ol

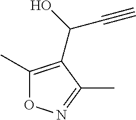

### a) 1-(3,5-dimethylisoxazol-4-yl)-3-(triisopropylsilyl)prop-2-yn-1-ol

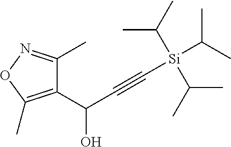 [SMILES: Cc1noc(C)c1C(O)C#C[Si](C(C)C)(C(C)C)C(C)C]

n-BuLi (48 mmol, 19 mL, 2.5 N in THF) was added to a solution of ethynyltriisopropylsilane (8 g, 44 mmol) in THF (30 mL) at 0° C. After stirring at 0° C. for 30 min, a solution of 3,5-dimethylisoxazole-4-carbaldehyde (2, 5 g, 40 mmol) in THF (20 mL) was added. The reaction mixture was further stirred for 2 h at 0° C., quenched with 2N HCl (24 mL), and extracted with EtOAc. The combined extracts were concentrated under reduced pressure, and the resulting residue was purified by flash column (petroleum ether/EtOAc=5/1) to give 1-(3,5-dimethylisoxazol-4-yl)-3-(triisopropylsilyl)prop-2-yn-1-ol (11 g, yield: 92%) as an oil. LCMSA039: 308[M+H]+; Rt: 1.98 min

### b) 1-(3,5-dimethylisoxazol-4-yl)prop-2-yn-1-ol

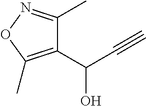

TBAF (19.7 mmol, 19.7 mL, 1N in THF) was added to a solution of 1-(3,5-dimethylisoxazol-4-yl)-3-(triisopropylsilyl)prop-2-yn-1-ol (5.5 g, 179 mmol) in THF (50 mL) at 0° C. After stirring at 0° C. for 2 h, the reaction mixture was extracted with EtOAc. The combined extracts were dried over Na₂SO₄, and concentrated under reduced pressure. The resulting residue was purified by flash column (petroleum ether/EtOAc=2/1) to give 1-(3,5-dimethylisoxazol-4-yl)prop-2-yn-1-ol (2.45 g, yield: 90%) as an oil. LCMSA036: 152.1[M+H]+; Rt: 1.21 min.

### Preparation 2

### (S)-2-(2-((3,5-dimethylisoxazol-4-yl)(hydroxy)methyl)benzofuran-5-yl)acetic acid

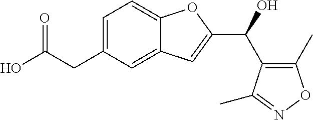 [SMILES: Cc1noc(C)c1[C@H](O)c1cc2cc(CC(=O)O)ccc2o1]

### a) (S)-1-(3,5-dimethylisoxazol-4-yl)-3-(triisopropylsilyl)prop-2-yn-1-ol

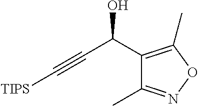

(Triisopropylsilyl) acetylene (219 g, 1.2 mol) was added to a diethylzinc solution (1.1 L, 1.02 Kg, 1.0M in toluene, 1.1 mol.) at ˜20-25° C. and then refluxed for 5 h. The reaction was cooled to ˜20-25° C. and (R)-Binol (55.2 g, 192 mmol) in CH₂Cl₂ (3.6 L) was added. The reaction mixture was stirred for 1 h, and then Ti(OⁱPr)₄ (158 g, 0.48 mol) was added. Then the mixture was stirred for another 1 h and a solution of 3,5-dimethylisoxazole-4-carbaldehyde (60 g, 0.48 mol, 1.0 eq) in CH₂Cl₂ (1.1 L) was added. The solution was stirred for 10 min, and then at rt an additional 16 h. Sat. NH4Cl solution (3 L) was added to the reaction and the mixture was filtered through a pad of Celite (500 g). The filter cake was washed with CH₂Cl₂ (2.1 L). The combined organic layers were washed with water (1 L), and brine (1 L), and concentrated under reduced pressure at ˜60-70° C. to give about 500 mL of a pale yellow liquid, which was treated with 1 L of n-heptane. A solid precipitated, which was filtered, and the filtrate was concentrated under reduced pressure at ˜60-70° C. to give about 300 mL of a pale yellow liquid (297 g, crude).

### b) (R)-1-(3,5-dimethylisoxazol-4-yl)prop-2-yn-1-ol

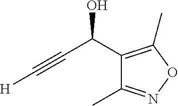

This intermediate was synthesized from (S)-1-(3,5-dimethylisoxazol-4-yl)-3-(triisopropylsilyl)prop-2-yn-1-ol essentially as described in preparation 22 (b) (45 g, yield: 62% for 2 steps).

### c) methyl 2-(4-hydroxy-3-iodophenyl)acetate

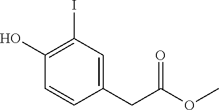

Methyl 2-(4-hydroxyphenyl)acetate (10.0 g, 0.060 mol) and NaI (10.0 g, 0.066 mol) were dissolved in DMF (50.0 mL), and the solution was cooled to −10° C. NaClO aqueous (60 mL) was added drop wise to the reaction mixture while keeping the temperature below 5° C. The mixture was stirred at −5˜5° C. for 10-25 min, and then quenched with a solution of 10% NaHSO3 aq. (100 mL, aq.) with stirring while keeping the temperature below 5° C., The yellow solution was stirred for 10 min at ˜10° C. and then the pH was adjusted to ˜2-3 with 2N HCl aqueous (40 mL). The mixture was extracted with EtOAc (100 mL, 50 mL, 50 mL), The combined extracts were washed with 10% NaHSO3 aq. (20 mL×2) and brine (20 mL*2), dried over Na2SO4 overnight, and concentrated under reduced pressure to give methyl 2-(4-hydroxy-3-iodophenyl)acetate (15.4 g, yield: 87.5%)

### d) (S)-methyl 2-(2-((3,5-dimethylisoxazol-4-yl)(hydroxy)methyl)benzofuran-5-yl)acetate

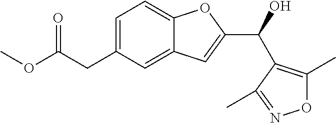

Methyl 2-(4-hydroxy-3-iodophenyl)acetate (78 g, 0.27 mol), Pd(PPh₃)₂Cl₂(1.85 g, 2.7 mmol), CuI (1 g, 5.3 mmol), and K₂CO₃ (68.5 g, 0.49 mmol) were added to 800 mL of EtOAc and the reaction was degassed. The mixture was heated to 50-60° C. and (R)-1-(3,5-dimethylisoxazol-4-yl)prop-2-yn-1-ol (50 g, 0.33 mol) in EtOAc (200 mL) was dropped by syringe over 1 h. Then the mixture was stirring at 50-60° C. for 3 h and LCMS showed compound 7-I was 3%. Then the reaction was continued to stir at 50-60° C. for another 1 h. The reaction was then filtered through a pad of Celite (50 g) and the filter cake was washed with EtOAc (500 mL). The combined filtrates were washed with water (500 mL) and brine (500 mL), and concentrated under reduced pressure to give (S)-methyl 2-(2-((3,5-dimethylisoxazol-4-yl)(hydroxy)methyl)benzofuran-5-yl)acetate, which was carried through without further purification.

### e) (S)-2-(2-((3,5-dimethylisoxazol-4-yl)(hydroxy)methyl)benzofuran-5-yl)acetic acid

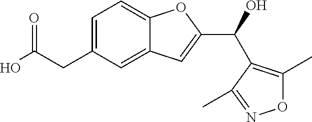 [SMILES: Cc1noc(C)c1[C@H](O)c1cc2cc(CC(=O)O)ccc2o1]

(S)-methyl 2-(2-((3,5-dimethylisoxazol-4-yl)(hydroxy)methyl)benzofuran-5-yl)acetate from step (d) was dissolved in 200 mL of THF at 15˜25° C. A solution of LiOH—H2O (17 g, 0.41 mol) in 50 mL of water was added and the mixture was stirred at 30±5° C. for 2 h. The mixture was then washed with EtOAc (30 mL*3) after 30.0 mL of water was added at rt. The organics were washed with water (30 mL*2). The combined aqueous layers were neutralized to pH˜2-4 with 1N HCl aq. (˜30 mL) while a lot of pale yellow solid appeared. The aqueous was extracted with EtOAc (3*40 mL) and the organics were washed with water (20 mL) and brine (20 mL). The organics was concentrated under reduced pressure (<40° C.) to give yellow solid which was slurried in 25.0 mL of MTBE overnight at rt (10˜15° C.). and then filtered, washed with MTBE (10 mL×2) and dried to give (S)-2-(2-((3,5-dimethylisoxazol-4-yl)(hydroxy)methyl)benzofuran-5-yl)acetic acid as a pale yellow solid (6.2 g, yield: 65%).

### Example 1

### 2-(2-((3,5-dimethylisoxazol-4-yl)(hydroxy)methyl)benzofuran-5-yl)-N-(1-(2-methoxy-4-methylphenyl)-4-methylpentyl)acetamide

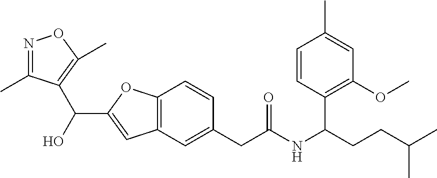 [SMILES: COc1cc(C)ccc1C(CCC(C)C)NC(=O)Cc1ccc2oc(C(O)c3c(C)noc3C)cc2c1]

### (a) methyl 2-(4-hydroxyphenyl)acetate

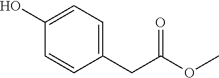

To a solution of 2-(4-hydroxyphenyl)acetic acid (100 g, 658 mmol) in MeOH (1000 mL) was added dropwise conc. aq. H₂SO₄ (40 mL) at 0° C. and the reaction was heated at 80° C. overnight. After completion of the reaction, the reaction mixture was cooled to rt and MeOH was removed under reduced pressure. The residue obtained was dissolved in water (500 mL) and neutralized (pH=7) using saturated aq. NaHCO₃. The aqueous layer was extracted with EtOAc (3×500 mL). The combined organic layers were dried over Na₂SO₄ and removal of solvent provided the title compound (101 g, 92%) as pale yellow solid. LCMS-P1: 167.2 [M+H]⁺; Rₜ=1.276 min.

### (b) methyl 2-(3-formyl-4-hydroxyphenyl)acetate

To a solution of methyl 2-(4-hydroxyphenyl)acetate (30 g, 180 mmol) in CH₃CN (150 mL) was added MgCl₂ (33.8 g, 360 mmol) and Et₃N (72.6 g, 720 mmol) under nitrogen and the mixture was refluxed for 1 h. Then (CH₂O)ₙ was added and the reaction was refluxed overnight. After cooling to rt, Et₂O (200 mL) and 1M HCl (300 mL) were added. The organic layer was separated and washed with 1M aq. HCl (3×300 mL), and dried over Na₂SO₄. After removal of solvent, the residue was purified by silica gel column chromotography (petroleum ether/EtOAc=5/1) to obtain the title compound (21.6 g, yield=62%) as a yellow oil. LCMS-P1: 195 [M+H]⁺; Rₜ=1.352 min.

### (c) methyl 2-(3-(2,2-dibromovinyl)-4-hydroxyphenyl)acetate

To a solution of CBr₄ (23 g, 70 mmol) in CH₂Cl₂ (100 mL) was added a solution of PPh₃ (27.5 g, 105 mmol) in CH₂Cl₂ (50 mL) at 0° C. and reaction was stirred for 15 min at the same temperature. Then a solution of methyl 2-(3-formyl-4-hydroxyphenyl)acetate (7 g, 35 mmol) in CH₂Cl₂ (25 mL) and Et₃N (10.6 g, 105 mmol) was added at 0° C. during 1 h. After the addition, the reaction mixture was stirred for an additional 2 h. Water (150 mL) was added to the reaction mixture slowly, followed by extraction with CH₂Cl₂ (3×200 mL). The organic layer was dried over Na₂SO₄ and concentrated to obtain a crude product. The residue was purified by silica gel column chromotography (petroleum ether/EtOAc=10/1) to obtain the title compound (4.6 g, yield: 38%) as a yellow oil. LCMS-P1: 349 [M+H]⁺; Rₜ=1.625 min.

### (d) methyl 2-(2-bromobenzofuran-5-yl)acetate

To a solution of methyl 2-(3-(2,2-dibromovinyl)-4-hydroxyphenyl)acetate (2.7 g, 7.75 mmol) in THF (200 mL) were added K₃PO₄ (3.28 g, 15.5 mmol) and CuI (59 mg, 0.31 mmol) under nitrogen. The reaction mixture was stirred at 80° C. for 12 h. After completion of the reaction, water (100 mL) was added to the reaction mixture, followed by extraction with CH₂Cl₂ (3×100 mL). The combined organic layers were dried over Na₂SO₄ and concentrated to obtain a crude product. The residue was purified by silica gel column chromotography (petroleum ether/EtOAc=50/1) to obtain the title compound (1.525 g, yield: 73%) as a yellow solid. LCMS-P1: 269 [M+H]⁺; Rₜ=1.670 min. ¹H NMR (400 MHz, DMSO-d₆) δ ppm 7.53-7.55 (d, 1H), 7.497-7.499 (d, 1H), 7.19-7.22 (d, 1H), 7.11 (s, 1H), 3.77 (s, 2H), 3.61 (s, 3H).

### (e) 1-(2-methoxy-4-methylphenyl)-4-methylpentan-1-amine

A catalytic amount of I₂ and around 0.2 gram of 1-bromo-3-methylbutane were added to a suspension of Mg (0.36 g, 15 mmol) in THF (30 mL). The reaction was initiated by heating, and the remained 1-bromo-3-methylbutane (1.8 g, 12 mmol) was added drop-wise. The mixture was stirred at rt for 4 h under nitrogen. 2-Methoxy-4-methylbenzonitrile (100 mg, 0.68 mmol) was added dropwise into the solution. After the addition, the reaction mixture was refluxed for 12 h. The reaction mixture was then cooled to rt and quenched by addition 5 mL of MeOH. NaBH₄ (31 mg, 0.82 mmol) and MeOH (10 mL) were then added to the reaction mixture. After stirring at rt for 2 h, the reaction was quenched by the addition of water (20 mL). Solvent was removed under reduced pressure. The residue was extracted with EtOAc (200 mL×3). The combined extracts were concentrated under reduced pressure and the resulting residue was purified by pre-TLC using 10% MeOH in CH₂Cl₂ to give 1-(2-methoxy-4-methylphenyl)-4-methylpentan-1-amine as a yellow solid (60 mg, Yield: 40%). LC-MSA024: 205.2 [M−NH2]⁺; Rt=1.33 min, Purity 57.62%(254 nm).

### (f) methyl 3,5-dimethylisoxazole-4-carboxylate

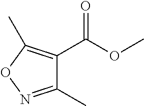

To a solution of 3,5-dimethylisoxazole-4-carboxylic acid (9.2 g, 65.2 mmol) in MeOH (50 mL) was added SOCl₂ (15.3 g, 130.4 mmol) slowly. The resulting mixture was heated to 70° C. overnight. When the reaction was complete, the reaction was cooled, concentrated, and purified by silica gel column chromotography (petroleum ether/EtOAc=10/1) to afford the title compound (9.0 g, yield: 89%). LCMS-P1: 156 [M+H]⁺; Rₜ=1.404 min.

### (g) (3,5-dimethylisoxazol-4-yl)methanol

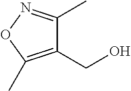

To a stirred solution of methyl 3,5-dimethylisoxazole-4-carboxylate (9.0 g, 58 mmol) in THF (200 mL) at 0° C. was added LiAlH₄ (2.42 g, 63.8 mmol) in portions. The reaction mixture was allowed to warm to rt and stirred overnight. The reaction was quenched with 2.5 mL water, 5 mL 10% aqueous NaOH solution, and 7.5 mL water successively. After filtration, the mixture was concentrated to afford the title compound (5.0 g, yield: 68%). LCMS-P1: 128 [M+H]⁺; Rₜ=0.963 min. ¹H NMR (400 MHz, CDCl₃,) δ ppm 4.37 (s, 2H), 2.30 (s, 3H), 2.20 (s, 3H).

### (h) 3,5-dimethylisoxazole-4-carbaldehyde

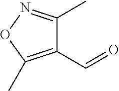

To a solution of (3,5-dimethylisoxazol-4-yl)methanol (1.00 g, 7.86 mmol) in CH₂Cl₂ (20 mL) at 0° C. was added Dess-Martin periodinane (4.17 g, 9.83 mmol) slowly within 10 min and the resulting mixture was warmed to rt. The reaction mixture was stirred at rt for 60 min. After completion of the reaction, the reaction mixture was filtered through Celite® and washed with CH₂Cl₂. The organic layer was dried over Na₂SO₄, concentrated, and purified by silica gel column chromatography (15% EtOAc/Hexanes) to provide the title compound (0.450 g, 45.73%). ¹H NMR (400 MHz, DMSO-d₆) δ ppm 9.92 (s, 1H), 2.68 (s, 3H), 2.37 (s, 3H).

### (i) methyl 2-(2-((3,5-dimethylisoxazol-4-yl)(hydroxy)methyl)benzofuran-5-yl)acetate

To a solution of methyl 2-(2-bromobenzofuran-5-yl)acetate (2.0 mg, 7.46 mmol) in 50 mL THF at 0° C. was added i-PrMgCl (5.6 mL, 11.2 mmol, 2N in THF). The mixture was stirred at 0° C. for 30 min. Then 3,5-dimethylisoxazole-4-carbaldehyde (1.5 g, 12 mmol) was added to the mixture. The resulting mixture was stirred for 2 h. Saturated aq. NH₄Cl (10 mL) was added to the mixture and the mixture was extracted with EtOAc (3×50 mL). The combined organic layers were washed with brine (3×15 mL) and dried over Na₂SO₄. The solvent was evaporated to a residue which was purified by silica gel column chromatography (30% EtOAc/petroleum ether) to afford the title compound (900 mg, yield 38%) as a yellow oil. LCMS-P1: 316 [M+H]⁺; Rₜ=1.481 min.

### (j) 2-(2-((3,5-dimethylisoxazol-4-yl)(hydroxy)methyl)benzofuran-5-yl)acetic acid

To a solution of methyl 2-(2-((3,5-dimethylisoxazol-4-yl)(hydroxy)methyl)benzofuran-5-yl)acetate (900 mg, 2.86 mmol) in THF (20 mL) and water (10 mL) was added LiOH (600.6 mg, 14.3 mmol). The mixture was stirred at rt for 1 h; then heated to 40° C. for 2 h. Then water (10 mL) was added to the mixture, and AcOH was used to adjust the aqueous phase to pH=6-7. The mixture was extracted with EtOAc (3×50 mL). The combined organic layers were washed with brine (3×15 mL), and dried over Na₂SO₄. The solvent was evaporated to give the title compound (650 mg, yield 75.6%) as a yellowish solid. The title compound was used in the next step without further purification. LCMS-P1: 302 [M+H]⁺; Rₜ=1.277 min.

### (k) 2-(2-((3,5-dimethylisoxazol-4-yl)(hydroxy)methyl)benzofuran-5-yl)-N-(1-(2-methoxy-4-methylphenyl)-4-methylpentyl)acetamide

1-(2-Methoxy-4-methylphenyl)-4-methylpentan-1-amine (25 mg, 0.11 mmol), EDCI (42 mg, 0.22 mmol), HOBt (30 mg, 0.22 mmol), and DIPEA (43 mg, 0.33 mmol) were added to a solution of 2-(2-((3,5-dimethylisoxazol-4-yl)(hydroxy)methyl)benzofuran-5-yl)acetic acid (36 mg, 0.12 mmol) in CH₂Cl₂ (10 mL). The reaction mixture was stirred at rt overnight. The reaction mixture was then diluted with water (30 mL) and extracted with EtOAc (30 mL×3). The combined organic solvents were washed with 1% HCl aqueous solution, dried over Na₂SO₄, and concentrated under reduced pressure. The resulting residue was purified by pre-TLC using 50% EtOAc in petroleum ether to give 2-(2-((3,5-dimethylisoxazol-4-yl)(hydroxy)methyl)benzofuran-5-yl)-N-(1-(2-methoxy-4-methylphenyl)-4-methylpentyl)acetamide as a white solid (7 mg, Yield: 12.7%). LC-MSA024: m/z 505.3 [M+H]⁺; Rt=1.81 min. Purity: 98.92%. ¹H NMR (MeOD, 400 MHz): δ 7.49 (s, 1H), 7.39 (d, J=8.4 Hz, 1H), 7.20 (dd, J=8.0, 4.0 Hz 1H), 7.03 (d, J=8.0 Hz, 1H), 6.72-6.67 (m, 3H), 5.88 (s, 1H), 5.05-5.01 (m, 1H), 3.66 (s, 2H), 3.56 (s, 3H), 2.38 (s, 3H), 2.30 (s, 3H), 2.21 (s, 3H), 1.72-1.64 (m, 2H), 153-1.51 (m, 1H), 1.10-1.08 (m, 2H), 0.90-0.84 (m, 6H).

### Example 2

### N-((4-chlorophenyl)(phenyl)methyl)-2-(2-((3,5-dimethylisoxazol-4-yl)(hydroxy)methyl)benzofuran-5-yl)-2-methylpropanamide

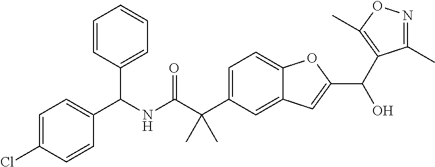 [SMILES: Cc1noc(C)c1C(O)c1cc2cc(C(C)(C)C(=O)NC(c3ccccc3)c3ccc(Cl)cc3)ccc2o1]

### (a) methyl 2-(2-bromobenzofuran-5-yl)-2-methylpropanoate

To a stirred solution of methyl 2-(2-bromobenzofuran-5-yl)acetate (1.0 g, 3.7 mmol) in 100 mL of THF was added potassium tert-butoxide (1.25 g, 11.2 mmol) and 18-Crown-6 (50 mg) under nitrogen. The resulting mixture was stirred at rt for 30 min. Then iodomethane (1.3 g, 9.25 mmol) was added and the mixture was stirred at rt for 3 h. EtOAc (100 mL) was added. The organic phase was washed with saturated NH₄Cl (3×100 mL) and brine (3×100 mL) and dried over Na₂SO₄. After removal of solvent, the residue was purified by silica gel column chromotography (petroleum ether/EtOAc=10/1) to obtain the title compound (376 mg, yield: 34%) as a white solid. LCMS-P1: 297 [M+H]⁺; Rₜ=1.250 min. ¹H NMR (400 MHz, CDCl₃) δ ppm 7.48 (d, J=3.2 Hz, 1H), 7.40 (d, J=8.8 Hz, 1H), 7.27-7.26 (m, 1H), 6.71 (d, J=0.4 Hz, 1H), 3.66 (s, 3H), 1.63 (s, 6H).

### (b) methyl 2-(2-((3,5-dimethylisoxazol-4-yl)(hydroxy)methyl)benzofuran-5-yl)-2-methylpropanoate

To a solution of methyl 2-(2-bromobenzofuran-5-yl)-2-methylpropanoate (310 mg, 1.05 mmol) in THF (10 mL) at 0° C. was added i-PrMgCl (0.79 mL, 1.57 mmol, 2N in THF). The mixture was stirred at 0° C. for 30 min. Then 3,5-dimethylisoxazole-4-carbaldehyde (196 mg, 1.57 mmol) was added to the mixture and the resulting mixture was stirred for 2 h. Saturated aq. NH₄Cl (10 mL) was added to the mixture, and the mixture was extracted with EtOAc (3×20 mL). The combined organic layers were washed with brine (15 mL), and dried over Na₂SO₄. The solvent was evaporated to residue which was purified by silica gel column chromatography (30% EtOAc/petroleum ether) to afford the title compound (190 mg, yield 53%) as a colorless oil. LCMS-P1: 344 [M+H]⁺; Rₜ=1.525 min.

### (c) 2-(2-((3,5-dimethylisoxazol-4-yl)(hydroxy)methylbenzofuran-5-yl)-2-methylpropanoic acid

To a solution of methyl 2-(2-((3,5-dimethylisoxazol-4-yl)(hydroxy)methyl)benzofuran-5-yl)-2-methylpropanoate (240 mg, 0.7 mmol) in THF (5 mL) and water (1 mL) was added LiOH (50 mg, 2 mmol). The mixture was stirred at rt overnight. Then water (10 mL) was added to the mixture, and AcOH was used to adjust the aqueous phase to pH=6-7. The mixture was extracted with EtOAc (3×15 mL). The combined organic layers were washed with brine (15 mL) and dried over Na₂SO₄. The solvent was evaporated to give the title compound (200 mg, yield 86%) as a yellowish solid. The acid was used in the next step without further purification. LCMS-P1: 330 [M+H]⁺; Rₜ=1.400 min.

### (d) N-((4-chlorophenyl)(phenyl)methyl)-2-(2-((3,5-dimethylisoxazol-4-yl)(hydroxy)methyl)benzofuran-5-yl)-2-methylpropanamide

To a stirred solution of 2-(2-((3,5-dimethylisoxazol-4-yl)(hydroxy)methyl)benzofuran-5-yl)-2-methylpropanoic acid (33 mg, 0.1 mmol) in CH₂Cl₂ (10 mL) was added HOBt (18 mg, 0.13 mmol), EDC (25 mg, 0.13 mmol), DIPEA (52 mg, 0.4 mmol) and (4-chlorophenyl)(phenyl)methanamine (33 mg, 0.13 mmol). The resulting mixture was stirred at rt overnight. The mixture was washed with diluted HCl (3×20 mL) and brine (3×20 mL) and then dried over Na₂SO₄. After removal of solvent, the residue was purified by preparatory TLC on silica gel (CH₂Cl₂/EtOAc=2/1) to obtain the title compound (11 mg, yield: 21%) as a white solid. LCMS-P1: 529 [M+H]⁺; Rₜ=1.763 min. ¹H NMR (400 MHz, DMSO-d₆) δ ppm 8.11 (d, J=8.8 Hz, 1H), 7.48 (s, 1H), 7.35 (d, J=8.4 Hz, 1H), 7.31-7.08 (m, 10H), 6.72 (s, 1H), 6.20-6.18 (m, 2H), 5.82 (d, J=3.6 Hz, 1H), 2.33 (s, 3H), 2.19 (s, 3H), 2.10 (s, 3H), 1.51 (s, 3H).

### Example 3

### N-((2-((3,5-dimethylisoxazol-4-yl)(hydroxy)methyl)benzofuran-5-yl)methyl)-2-(2,4-dimethylphenyl)-2-phenylacetamide

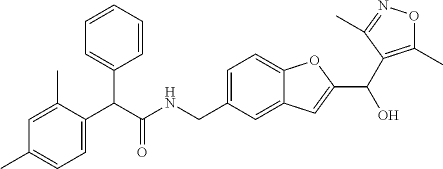

### (a) 2-(2,4-dimethylphenyl)-2-phenylacetic acid

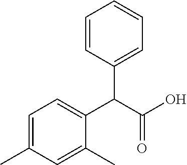 [SMILES: Cc1ccc(C(C(=O)O)c2ccccc2)c(C)c1]

A solution of mandelic acid (10.0 g, 65.7 mmol) in m-xylene (56.79 mL, 460 mmol) was heated to 60-70° C. followed by addition of SnCl₄ (11.5 mL, 98.6 mmol) over 2 h. After addition, the reaction mixture was cooled to rt and then stirred for 6 h at rt. The completion of the reaction was monitored by TLC on silica gel using hexanes:EtOAc (1:1) as the mobile phase. After completion of the reaction, ice-water (100 mL) was added into the reaction mixture, and the mixture was extracted with Et₂O (3×250 mL). The combined Et₂O layers were discarded. The aqueous layer was treated with 8% aq. Na₂CO₃ (10×50 mL) and the combined aqueous layers was then acidified using 6 N HCl (20 mL) and the solid obtained was filtered and dried. The crude solid product was purified using silica gel column chromatography using 20% EtOAc:hexanes to obtain the title compound (6.25 g, 39.63%) as a solid. ¹H NMR (400 MHz, DMSO-d₆) δ ppm 12.6 (s, 1H), 7.21-7.33 (m, 6H), 7.09-7.12 (m, 1H), 6.97-7.06 (m, 3H), 5.12 (s, 1H), 2.23 (s, 3H), 2.17-2.18 (d, 4H).

### (b) tert-butyl((2-((3,5-dimethylisoxazol-4-yl)(hydroxy)methyl)benzofuran-5-yl)methyl)carbamate

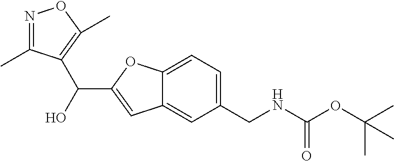 [SMILES: Cc1noc(C)c1C(O)c1cc2cc(CNC(=O)OC(C)(C)C)ccc2o1]

To a solution of 2-(2-((3,5-dimethylisoxazol-4-yl)(hydroxy)methyl)benzofuran-5-yl)acetic acid (301 mg, 1 mmol) in tert-butanol (10 mL) was added Et₃N (202 mg, 2 mmol) and diphenyl phosphorazidate (330 mg, 1.2 mmol). The mixture was heated to 80° C. under argon overnight. After cooling to rt, the solvent was removed under reduced pressure. The residue was purified by silica gel column chromatography with petroleum ether:EtOAc=3:1 to afford the title compound as a colorless oil (70 mg, yield 18.8%). LCMS-P1: 373.2 [M+H]⁺;

### (c) (5-(aminomethyl)benzofuran-2-yl)(3,5-dimethylisoxazol-4-yl)methanol

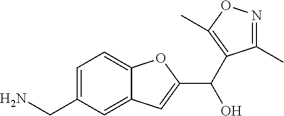 [SMILES: Cc1noc(C)c1C(O)c1cc2cc(CN)ccc2o1]

To a solution of tert-butyl ((2-((3,5-dimethylisoxazol-4-yl)(hydroxy)methyl)benzofuran-5-yl)methyl)carbamate (70 mg, 0.18 mmol) in MeOH (5 mL) was added HCl (sat.) in MeOH (0.5 mL). After stirring at rt for 30 min, the solvent was removed under reduced pressure to afford crude the title compound (35 mg), which was used directly for the next step without further purification.

### (d) N-((2-((3,5-dimethylisoxazol-4-yl)(hydroxy)methyl)benzofuran-5-yl)methyl)-2-(2,4-dimethylphenyl)-2-phenylacetamide

This compound was synthesized from (5-(aminomethyl)benzofuran-2-yl)(3,5-dimethylisoxazol-4-yl)methanol and 2-(2,4-dimethylphenyl)-2-phenylacetic acid as example 1(k) and the compound was purified by preparatory TLC on silica gel to provide the title compound (10 mg, Yield: 11%). LCMS-P1: 495.0 [M+H]⁺; Rₜ=1.78 min. ¹H NMR (400 MHz, DMSO-d₆) δ ppm 8.68 (t, J=5.6 Hz, 1H), 7.47-7.42 (m, 2H), 7.30-7.11 (m, 7H), 6.70-6.93 (m, 2H), 6.72 (s, 1H), 6.18 (d, J=4.4 Hz, 1H), 5.83 (d, J=4.4 Hz, 1H), 5.10 (s, 1H), 4.40-4.34 (m, 2H), 2.33 (s, 3H), 2.22 (s, 3H), 2.15 (s, 3H), 2.10 (s, 3H).

### Example 4

### 2-(2-(amino(3,5-dimethylisoxazol-4-yl)methyl)benzofuran-5-yl)-N-((2,4-dimethylphenyl)(phenyl)methyl)acetamide

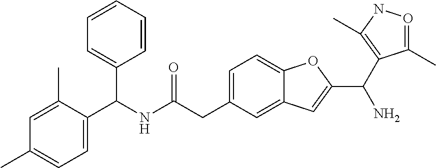

### (a) 2-(2-(chloro(3,5-dimethylisoxazol-4-yl)methyl)benzofuran-5-yl)-N-((2,4-dimethylphenyl)(phenyl)methyl)acetamide

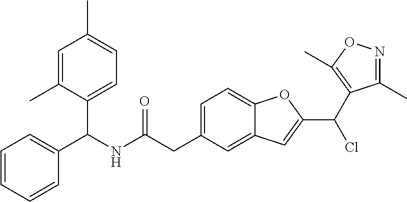 [SMILES: Cc1ccc(C(NC(=O)Cc2ccc3oc(C(Cl)c4c(C)noc4C)cc3c2)c2ccccc2)c(C)c1]

To a solution of 2-(2-((3,5-dimethylisoxazol-4-yl)(hydroxy)methyl)benzofuran-5-yl)-N-((2,4-dimethylphenyl)(phenyl)methyl)acetamide (70 mg, 0.14 mmol) in CH₂Cl₂ (10 mL) at 0° C. was added SOCl₂ (0.02 mL). The reaction mixture was stirred at rt for 2 h followed by concentration under reduced pressure to afford the title compound (70 mg, 72%).

### (b) 2-(2-(amino(3,5-dimethylisoxazol-4-yl)methyl)benzofuran-5-yl)-N-((2,4-dimethylphenyl)(phenyl)methyl)acetamide

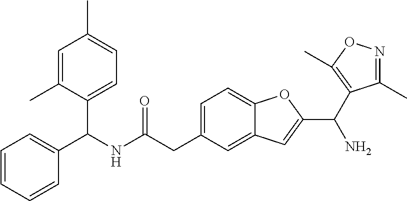 [SMILES: Cc1ccc(C(NC(=O)Cc2ccc3oc(C(N)c4c(C)noc4C)cc3c2)c2ccccc2)c(C)c1]

A mixture of 2-(2-(chloro(3,5-dimethylisoxazol-4-yl)methyl)benzofuran-5-yl)-N-((2,4-dimethylphenyl)(phenyl)methyl)acetamide (20 mg, 0.04 mmol) in an aqueous ammonia solution (5 mL) was stirred at rt for 3 h. The mixture was extracted with CH₂Cl₂ (3×20 mL). The combined organic layers were washed with water and dried over Na₂SO₄. The organic solvent was concentrated to residue which was purified by preparatory HPLC using 10-100% water/acetonitrile with 0.1% TFA to obtain the title compound (8 mg, 41%). LCMS-P1: 494 [M+H]⁺; Rₜ=1.462 min. ¹H NMR (500 MHz, CDCl₃) δ ppm 7.25-6.98 (m, 8H), 6.87-6.15 (m, 6H), 5.31 (s, 1H), 3.47 (s, 2H), 2.17 (s, 6H), 2.05 (s, 3H), 1.99 (s, 3H).

Using essentially the same procedure as described in example 1, the following compounds in table 1 were made.

TABLE 1

| Ex. | Structure/Name | NMR | LCMS |
|---|---|---|---|
| 5 | 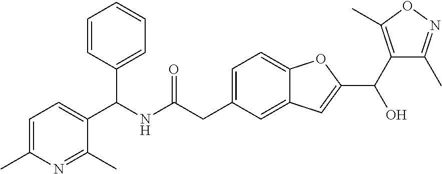 | ¹H NMR (400 MHz, CDCl₃) δ ppm 7.42 (d, J = 9.2 Hz, 2H), 7.26 (t, J = 4.4 Hz, 4H), 7.18 (d, J = 8.4 Hz, 1H), 7.11 (d, J = 7.6 Hz, 1H), 7.03 (t, J = 2.4 Hz, 2H), 6.90 (d, J = 7.6 Hz, 1H), 6.55 (s, 1H), 6.31 (d, J = 7.6 Hz, 1H), 5.87 (s, 2H), 3.71 (s, 2H), 2.49 (s, 3H), 2.39 (s, 6H), 2.25 (d, J = 0.4 Hz, 3H). | LCMS-P1: 496.0 [M + H]⁺; Rₜ = 1.31 min |
| | 2-(2-((3,5-dimethylisoxazol-4- | | |
| | yl)(hydroxy)methyl)benzo- | | |
| | furan-5-yl)-N-((2,6- | | |
| | dimethylpyridin-3- | | |
| | yl)(phenyl)methyl) acetamide | | |
| 6 | 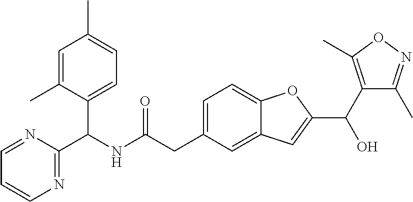 [SMILES: Cc1ccc(C(NC(=O)Cc2ccc3oc(C(O)c4c(C)noc4C)cc3c2)c2ncccn2)c(C)c1] | ¹H NMR (400 MHz, CDCl₃) δ ppm 8.61 (d, J = 4.8 Hz, 1H), 7.46 (s, 1H), 7.40 (d, J = 8.8 Hz, 1H), 7.29 (d, J = 7.2 Hz, 1H), 7.21 (dd, J1 = 2 Hz, J2 = 8.8 Hz, 1H), 7.12 (t, J = 4.8 Hz, 1H), 6.96 (s, 1H), 6.85-6. 88 (m, 2H), 6.54 (s, 1H), 6.45 (d, J = 7.6 Hz, 1H), 5.77 (s, 1H), 5.87 (s, 2H), 3.70 (s, 2H), 2.55 (s, 3H), 2.39 (s, 3H), 2.26 (s, 3H), 2.23 (s, 3H). | LCMS-P1: 497.0 [M + H]⁺; Rₜ = 1.61 min |
| | 2-(2-((3,5-dimethylisoxazol-4- | | |
| | yl)(hydroxy)methyl)benzo- | | |
| | furan-5-yl)-N-((2,4- | | |
| | dimethylphenyl)(pyrimidin-2- | | |
| | yl)methyl)acetamide | | |
| 7 | 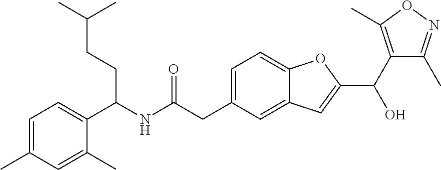 | ¹H NMR (400 MHz, MeOH-d₄) δ ppm 7.47 (s, 1H), 7.35 (m, 1H), 7.15 (m, 2H), 6.94 (m, 2H), 6.68 (s, 1H), 5.87 (s, 1H), 4.88 (t, 1H), 3.57 (d, J = 5.6 Hz, 2H), 2.37 (s, 3H), 2.29 (s, 3H), 2.26 (s, 3H), 2.20 (s, 3H), 1.70 (m, 2H), 1.51 (m, 1H), 1.29 (m, 2H), 0.83 (m, 6H). | LCMS-P1: 489.3 [M + H]⁺; Rₜ = 1.78 min |
| | 2-(2-((3,5-dimethylisoxazol-4- | | |
| | yl)(hydroxy)methyl)benzo- | | |
| | furan-5-yl)-N-(1-(2,4- | | |
| | dimethylphenyl)-4- | | |
| | methylpentyl)acetamide | | |
| 8 | 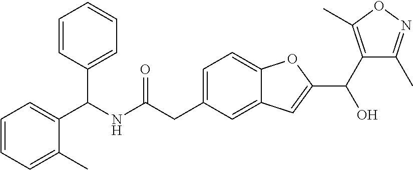 [SMILES: Cc1ccccc1C(NC(=O)Cc1ccc2oc(C(O)c3c(C)noc3C)cc2c1)c1ccccc1] | ¹H NMR (400 MHz, DMSO-d₆) δ ppm 8.954 (d, J = 6.8 Hz, 1H), 7.477 (s, 1H), 7.434 (d, J = 6.8 Hz, 1H), 7.320 (t, J = 6.0 Hz, 1H), 7.260 (d, J = 6.0 Hz, 1H), 7.125-7.192 (m, 6H), 7.122 (s, 1H), 6.746 (s, 1H), 6.199- 6.237 (m, 2H), 5.835 (d, J = 3.2 Hz, 1H), 3.594 (s, 2H), 2.346 (s, 3H), 2.190 (s, 3H), 2.121 (s, 3H). | LCMS-P1: 481.0 [M + H]⁺; Rₜ = 1.70 min |
| | 2-(2-((3,5-dimethylisoxazol-4- | | |
| | yl)(hydroxy)methyl)benzo- | | |
| | furan-5-yl)-N-(phenyl(o- | | |
| | tolyl)methyl)acetamide | | |
| 9 | 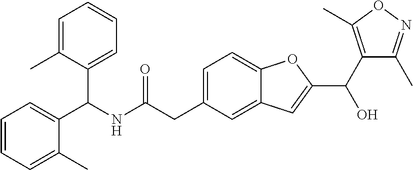 [SMILES: Cc1ccccc1C(NC(=O)Cc1ccc2oc(C(O)c3c(C)noc3C)cc2c1)c1ccccc1C] | ¹H NMR (400 MHz, MeOH-d₄) δ ppm 7.52 (d, J = 1.2 Hz, 1H), 7.37 (d, J = 8.4 Hz, 1H), 7.37-6.98 (m, 9H), 6.70 (s, 1H), 6.36 (s, 1H), 5.88 (s, 1H), 3.66 (s, 2H), 2.37 (s, 3H), 2.21 (s, 3H), 2.15 (s, 3H). | LCMS-P1: 495 [M + H]⁺; Rₜ = 1.98 min |
| | N-(di-o-tolylmethyl)-2-(2-((3,5- | | |
| | dimethylisoxazol-4- | | |
| | yl)(hydroxy)methyl)benzo- | | |
| | furan-5-yl)acetamide | | |
| 10 | 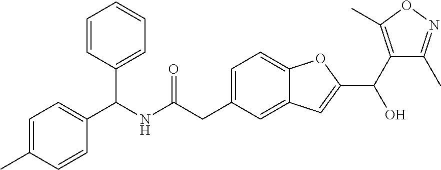 | ¹H NMR (400 MHz, MeOH-d₄) δ ppm 7.50 (d, J = 1.2 Hz, 1H), 7.37 (d, J = 8.4 Hz, 1H), 7.20-7.36 (m, 6H), 7.07-7.19 (m, 4H), 6.69 (s, 1H), 6.13 (s, 1H), 5.88 (s, 1H), 3.68 (s, 2H), 2.37 (s, 3H), 2.30 (s, 3H), 2.20 (s, 3H). | LCMS-P1: 481 [M + H]⁺; Rₜ = 1.95 min |
| | 2-(2-((3,5-dimethylisoxazol-4- | | |
| | yl)(hydroxy)methyl)benzo- | | |
| | furan-5-yl)-N-(phenyl(p- | | |
| | tolyl)methyl)acetamide | | |
| 11 | 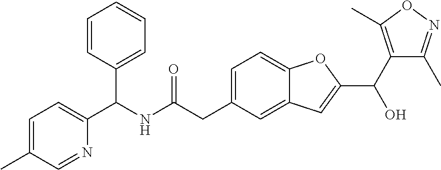 | ¹H NMR (400 MHz, MeOH-d₄) δ ppm 8.31 (d, J = 1.6 Hz, 1H), 7.58 (d, J = 2.0 Hz, 1H), 7.57 (d, J = 2.0 Hz, 1H), 7.51 (d, J = 1.2Hz, 1H), 7.28-7.21 (m, 7H), 6.70 (s, 1H), 6.14 (s, 1H), 5.88 (s, 1H), 5.50 (s, 1H), 3.71 (s, 2H), 2.35 (s, 3H), 2.20 (s, 3H), 2.05 (s, 3H). | LCMS-P1: 482 [M + H]⁺; Rₜ = 1.42 min |
| | 2-(2-((3,5-dimethylisoxazol-4- | | |
| | yl)(hydroxy)methyl)benzo- | | |
| | furan-5-yl)-N-((5-methylpyridin- | | |
| | 2-yl)(phenyl)methyl)acetamide | | |
| 12 | 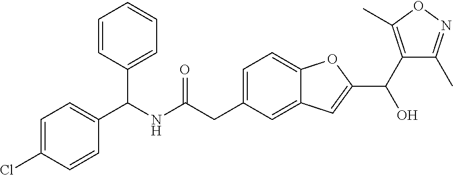 [SMILES: Cc1noc(C)c1C(O)c1cc2cc(CC(=O)NC(c3ccccc3)c3ccc(Cl)cc3)ccc2o1] | ¹H NMR (400 MHz, DMSO-d₆) δ ppm 9.04 (d, J = 8.8 Hz, 1H), 7.45- 7.15 (m, 12H), 6.73 (s, 1H), 6.19 (d, J = 4.8 Hz, 1H), 6.08 (d, J = 8.4 Hz, 1H), 5.81 (d, J = 4.0 Hz, 1H), 3.60 (s, 2H), 2.33 (s, 3H), 2.10 (s, 3H). | LCMS-P1: 501 [M + H]⁺; Rₜ = 1.664 min |
| | N-((4-chlorophenyl) | | |
| | (phenyl)methyl)2-(2-((3,5- | | |
| | dimethylisoxazol-4- | | |
| | yl)(hydroxy)methyl)benzo- | | |
| | furan-5-yl)acetamide | | |
| 13 | 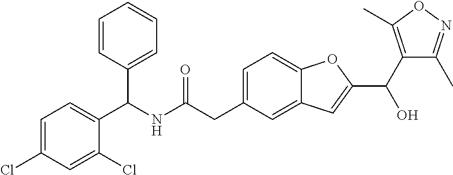 [SMILES: Cc1noc(C)c1C(O)c1cc2cc(CC(=O)NC(c3ccccc3)c3ccc(Cl)cc3Cl)ccc2o1] | ¹H NMR (400 MHz, DMSO-d₆) δ ppm 9.09 (d, J = 8.0 Hz, 1H), 7.60 (d, J = 1.6 Hz, 1H), 7.45-7.41 (m, 4H), 7.34-7.27 (m, 3H), 7.15 (d, J = 7.2 Hz, 3H), 6.74 (s, 1H), 6.32 (d, J = 8.0 Hz, 1H), 6.19 (d, J = 4.4 Hz, 1H), 5.82 (d, J = 4.0 Hz, 1H), 3.58 (s, 2H), 2.33 (s, 3H), 2.10 (s, 3H). | LCMS-P1: 535 [M + H]⁺; Rₜ = 1.719 min |
| | N-((2,4-dichlorophenyl) | | |
| | (phenyl)methyl)2-(2-((3,5- | | |
| | dimethylisoxazol-4- | | |
| | yl)(hydroxy)methyl)benzo- | | |
| | furan-5-yl)acetamide | | |
| 14 | 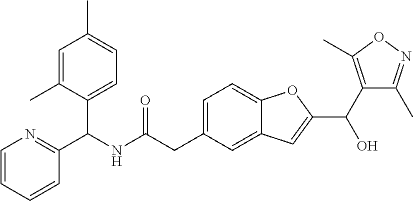 [SMILES: Cc1ccc(C(NC(=O)Cc2ccc3oc(C(O)c4c(C)noc4C)cc3c2)c2ccccn2)c(C)c1] | ¹H NMR (400 MHz, DMSO-d₆) δ ppm 8.90 (d, J = 8.0 Hz, 1H), 8.49 (d, J = 4.0 Hz, 1H), 7.75-7.14 (m, 6H), 6.95 (s, 1H), 6.90 (s, 2H), 6.72 (s, 1H), 6.22-6.18 (m, 2H), 5.81 (d, J = 3.6 Hz, 1H), 3.59 (s, 2H), 2.33 (s, 3H), 2.21 (s, 3H), 2.19 (s, 3H), 2.10 (s, 3H). | LCMS-P1: 496 [M + H]⁺; Rₜ = 1.469 min |
| | 2-(2-((3,5-dimethylisoxazol-4- | | |
| | yl)(hydroxy)methyl)benzo- | | |
| | furan-5-yl)-N-((2,4- | | |
| | dimethylphenyl)(pyridin-2- | | |
| | yl)methyl)acetamide | | |
| 15 | 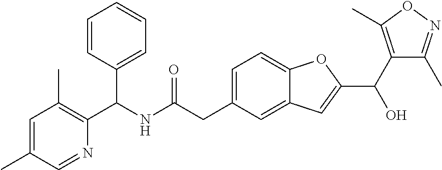 | ¹H NMR (400 MHz, DMSO-d₆) δ ppm 8.90 (d, J = 8.0 Hz, 1H), 7.45 (s, 1H), 7.41 (d, J = 8.4 Hz, 1H), 7.17 (d, J = 8.4 Hz, 1H), 7.11-7.07 (m, 7H), 6.72 (s, 1H), 6.18 (d, J = 4.4 Hz, 1H), 5.98 (d, J = 8.4 Hz, 1H), 5.81 (d, J = 4.0 Hz, 1H), 3.57 (s, 2H), 2.33 (s, 3H), 2.24 (s, 6H), 2.10 (s, 3H). | LCMS-P1: 495 [M + H]⁺; Rₜ = 1.695 min |
| | 2-(2-((3,5-dimethylisoxazol-4- | | |
| | yl)(hydroxy)methyl)benzo- | | |
| | furan-5-yl)-N-((3,5- | | |
| | dimethylpyridin-2- | | |
| | yl)(phenyl)methyl)acetamide | | |
| 16 | 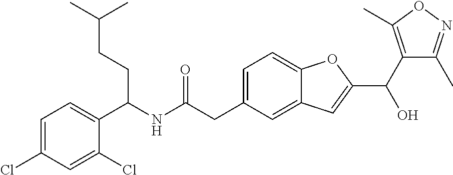 [SMILES: Cc1noc(C)c1C(O)c1cc2cc(CC(=O)NC(CCC(C)C)c3ccc(Cl)cc3Cl)ccc2o1] | ¹H NMR (400 MHz, DMSO-d₆) δ ppm 8.66 (d, J = 6 Hz, 1H), 7.54 (m, 5H), 7.16 (d, J = 6.4 Hz, 1H), 6.73 (s, 1H), 6.21 (s, 1H), 5.83 (s, 1H), 5.02 (s, 1H), 3.56 (m, 2H), 2.33 (s, 3H), 2.16 (s, 3H), 1.58 (s, 1H), 1.47 (s, 1H), 1.30 (m, 3H), 0.81 (m, 6H). | LCMS-P1: 529.0 [M + H]⁺; Rₜ = 1.90 min |
| | N-(1-(2,4-dichlorophenyl)-4- | | |
| | methylpentyl)-2-(2-((3,5- | | |
| | dimethylisoxazol-4- | | |
| | yl)(hydroxy)methyl)benzo- | | |
| | furan-5-yl)acetamide | | |
| 17 | 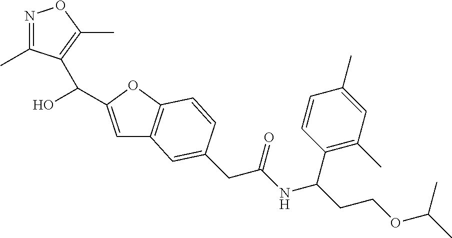 [SMILES: Cc1ccc(C(CCOC(C)C)NC(=O)Cc2ccc3oc(C(O)c4c(C)noc4C)cc3c2)c(C)c1] | 1H NMR (CDCl₃, 400 MHz): δ 7.36- 7.33 (m, 2H), 7.10 (d, J = 8.0 Hz, 1H), 6.86 (s, 1H), 6.83 (s, 2H), 6.75- 6.69 (m, 1H), 6.47 (s, 1H), 5.81 (s, 1H), 5.26-5.21 (m, 1H), 3.53 (s, 1H), 3.35-3.30 (m, 1H), 3.29-3.16 (m, 2H), 2.33 (s, 3H), 2.25 (s, 3H), 2.19 (s, 6H), 1.93-1.86 (m, 1H), 1.76-1.70 (m, 1H), 0.94-0.91 (m, 6H). | LCMS-A012: 505.2 [M + H]⁺; Rt = 1.46 min. |
| | 2-(2-((3,5-dimethylisoxazol-4- | | |
| | yl)(hydroxy)methyl)benzo- | | |
| | furan-5-yl)-N-(1-(2,4- | | |
| | dimethylphenyl)-3- | | |
| | isopropoxypropyl)acetamide | | |
| 18 | 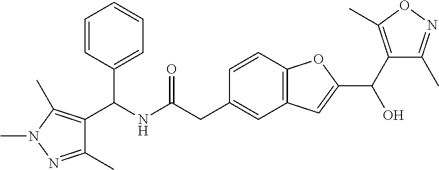 | ¹H NMR (MeOH-d₄, 400 MHz): δ 7.53 (s, 1H), 7.40 (d, J = 8.8 Hz, 1H), 7.29-7.26 (m, 4H), 7.17 (d, J = 7.6 Hz, 2H), 6.72 (s, 1H), 6.15 (s, 1H), 5.89 (s, 1H), 3.69 (s, 2H), 3.67 (s, 3H), 2.38 (s, 3H), 2.22 (s, 3H), 2.06 (s, 3H), 1.89 (s, 3H). | LCMS-A026: 499.1 [M + H]⁺; Rt = 1.58 min. |
| | 2-(2-((3,5-dimethylisoxazol-4- | | |
| | yl)(hydroxy)methyl)benzofuran- | | |
| | 5-yl)-N-(phenyl(1,3,5-trimethyl- | | |
| | 1H-pyrazol-4- | | |
| | yl)methyl)acetamide | | |
| 19 | 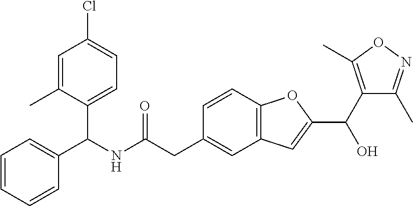 [SMILES: Cc1cc(Cl)ccc1C(NC(=O)Cc1ccc2oc(C(O)c3c(C)noc3C)cc2c1)c1ccccc1] | ¹H NMR (d₆-DMSO, 400 MHz): δ 8.96 (d, J = 8.0 Hz, 1H), 7.43 (t, J = 8.8 Hz, 2H), 7.33-7.09 (m, 9H), 6.73 (s, 1H), 6.20-6.16 (m, 2H), 5.82 (d, J = 4.0 Hz, 1H), 3.57 (s, 2H), 2.33 (s, 3H), 2.16 (s, 3H), 2.10 (s, 3H). | LC-MS: 515 [M + H]⁺; Rt = 1.699 min |
| | N-((4-chloro-2- | | |
| | methylphenyl)(phenyl)methyl)- | | |
| | 2-(2-((3,5-dimethylisoxazol-4- | | |
| | yl)(hydroxy)methyl)benzofuran- | | |
| | 5-yl)acetamide | | |
| 20 | 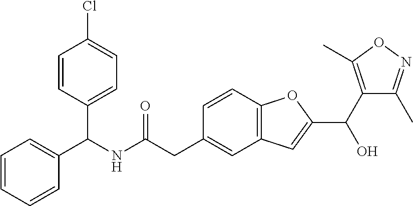 [SMILES: Cc1noc(C)c1C(O)c1cc2cc(CC(=O)NC(c3ccccc3)c3ccc(Cl)cc3)ccc2o1] | ¹H NMR (d₆-DMSO, 400 MHz): δ 9.04 (d, J = 8.8 Hz, 1H), 7.45-7.15 (m, 12H), 6.73 (s, 1H), 6.19 (d, J = 4.8 Hz, 1H), 6.08 (d, J = 8.4 Hz, 1H), 5.81 (d, J = 4.0 Hz, 1H), 3.60 (s, 2H), 2.33 (s, 3H), 2.10 (s, 3H). | LC-MS: 501 [M + H]⁺; Rt = 1.664 min. |
| | N-((4- | | |
| | chlorophenyl)(phenyl)methyl)- | | |
| | 2-(2-((3,5-dimethylisoxazol-4- | | |
| | yl)(hydroxy)methyl)benzofuran- | | |
| | 5-yl)acetamide | | |
| 21 | 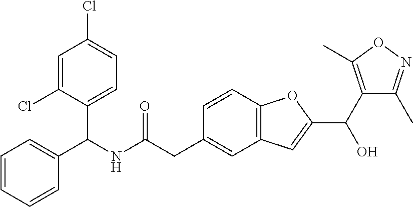 [SMILES: Cc1noc(C)c1C(O)c1cc2cc(CC(=O)NC(c3ccccc3)c3ccc(Cl)cc3Cl)ccc2o1] | ¹H NMR (d₆-DMSO, 400 MHz): δ 9.09 (d, J = 8.0 Hz, 1H), 7.60 (d, J = 1.6 Hz, 1H), 7.45-7.41 (m, 4H), 7.34-7.27 (m, 3H), 7.15 (d, J = 7.2 Hz, 3H), 6.74 (s, 1H), 6.32 (d, J = 8.0 Hz, 1H), 6.19 (d, J = 4.4 Hz, 1H), 5.82 (d, J = 4.0 Hz, 1H), 3.58 (s, 2H), 2.33 (s, 3H), 2.10 (s, 3H). | LC-MS: 535 [M + H]⁺; Rt = 1.719 min. |
| | N-((4- | | |
| | dichlorophenyl)(phenyl)methyl)- | | |
| | 2-(2-((3,5-dimethylisoxazol-4- | | |
| | yl)(hydroxy)methyl)benzofuran- | | |
| | 5-yl)acetamide | | |
| 22 | 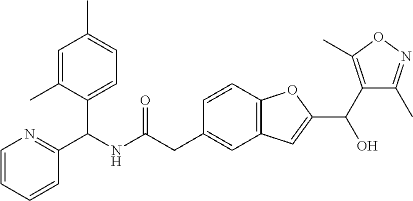 [SMILES: Cc1ccc(C(NC(=O)Cc2ccc3oc(C(O)c4c(C)noc4C)cc3c2)c2ccccn2)c(C)c1] | ¹H NMR (d₆-DMSO, 400 MHz): δ 8.90 (d, J = 8.0 Hz, 1H), 8.49 (d, J = 4.0 Hz, 1H), 7.75-7.14 (m, 6H), 6.95 (s, 1H), 6.90 (s, 2H), 6.72 (s, 1H), 6.22-6.18 (m, 2H), 5.81 (d, J = 3.6 Hz, 1H), 3.59 (s, 2H), 2.33 (s, 3H), 2.21 (s, 3H), 2.19 (s, 3H), 2.10 (s, 3H). | LC-MS: 496 [M + H]⁺; Rt = 1.469 min. |
| | 2-(2-((3,5-dimethylisoxazol-4- | | |
| | yl)(hydroxy)methyl)benzofuran- | | |
| | 5-yl)-N-((2,4- | | |
| | dimethylphenyl)(pyridin-2- | | |
| | yl)methyl)acetamide | | |
| 23 | 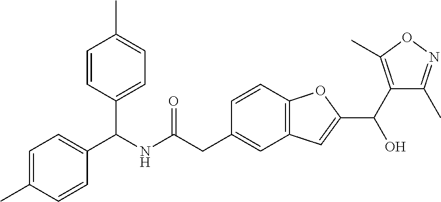 | ¹H NMR (d₆-DMSO, 400 MHz): δ 8.90 (d, J = 8.0 Hz, 1H), 7.45 (s, 1H), 7.41 (d, J = 8.4 Hz, 1H), 7.17 (d, J = 8.4 Hz, 1H), 7.11-7.07 (m, 8H), 6.72 (s, 1H), 6.18 (d, J = 4.4 Hz, 1H), 5.98 (d, J = 8.4 Hz, 1H), 5.81 (d, J = 4.0 Hz, 1H), 3.57 (s, 2H), 2.33 (s, 3H), 2.24 (s, 6H), 2.10 (s, 3H). | LC-MS: 495 [M + H]⁺; Rt = 1.695 min. |
| | N-(di-p-tolylmethyl)-2-(2-((3,5- | | |
| | dimethylisoxazol-4- | | |
| | yl)(hydroxy)methyl)benzofuran- | | |
| | 5-yl)acetamide | | |
| 24 | 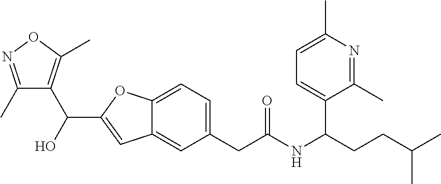 [SMILES: Cc1ccc(C(CCC(C)C)NC(=O)Cc2ccc3oc(C(O)c4c(C)noc4C)cc3c2)c(C)n1] | ¹H NMR (DMSO, 400 MHz): δ 8.53 (d, J = 6.4 Hz, 1H), 7.53 (d, J = 6.4 Hz, 1H), 7.44 (m, 3H), 7.14 (d, J = 6.8 Hz, 1H), 7.04 (d, J = 6.4 Hz, 1H), 6.72 (s, 1H), 6.24 (d, J = 3.6 Hz, 1H), 5.83 (d, J = 3.2 Hz, 1H), 4.85 (m, 1H), 2.41 (s, 3H), 2.36 (s, 3H), 2.33 (s, 3H), 2.11 (s, 3H), 1.60 (m, 2H), 1.46 (m, 1H), 1.25 (m, 2H), 0.86 (m, 6H). | LC-MS: m/z 490.1 [M + H]⁺; Rt = 1.27 min, purity 91.8%. |
| | 2-(2-((3,5-dimethylisoxazol-4- | | |
| | yl)(hydroxy)methyl)benzofuran- | | |
| | 5-yl)-N-(1-(2,6-dimethylpyridin- | | |
| | 3-yl)-4-methylpentyl)acetamide | | |
| 25 | 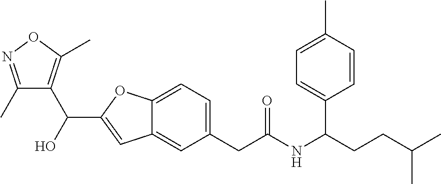 [SMILES: Cc1ccc(C(CCC(C)C)NC(=O)Cc2ccc3oc(C(O)c4c(C)noc4C)cc3c2)cc1] | ¹H NMR (DMSO, 400 MHz): δ 8.40 (d, J = 6.8 Hz, 1H), 7.44 (s, 1H), 7.41 (d, J = 6.8 Hz, 1H), 7.16-7.09 (m, 3H), 7.08 (d, J = 6.4 Hz, 2H), 6.72 (s, 1H), 6.18 (d, J = 3.6 Hz, 1H), 5.82 (d, J = 3.6 Hz, 1H), 4.70-4.68 (m, 1H), 3.50-3.33 (m, 2H), 2.33 (s, 3H), 2.25 (s, 3H), 2.11 (s, 3H), 1.62 (d, J = 6.0 Hz, 2H), 1.49-1.47 (m, 1H), 1.13-1.00 (m, 2H), 0.79-0.76 (m, 6H). | LC-MS024: m/z 475.7; [M + H]⁺; Rt = 1.785 min, purity 94.9%. |
| | 2-(2-((3,5-dimethylisoxazol-4- | | |
| | yl)(hydroxy)methyl)benzofuran- | | |
| | 5-yl)-N-(4-methyl-1-(p- | | |
| | tolyl)pentyl)acetamide | | |
| 26 | 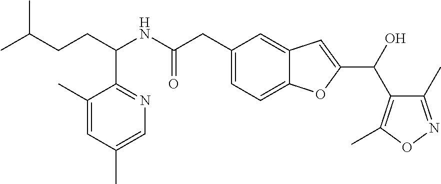 [SMILES: Cc1cnc(C(CCC(C)C)NC(=O)Cc2ccc3oc(C(O)c4c(C)noc4C)cc3c2)c(C)c1] | 1H NMR (DMSO-d6, 400 MHz): δ 8.33 (d, J = 8.4 Hz, 1H), 8.22 (s, 1H), 7.44-7.12 (m, 3H), 7.13 (d, J = 8.4 Hz, 1H), 6.71 (s, 1H), 6.18 (d, J = 4.4 Hz ,1H), 5.82 (t, J = 3.6 Hz, 1H), 5.02 (m, 1H), 3.55-3.41 (m, 2H), 2.50 (s, 3H), 2.25 (s, 3H), 2.23 (s, 3H), 2.11 (s, 3H), 1.76-1.64 (m, 2H), 1.46-1.40 (m, 1H), 1.12-0.92 (m, 2H), 0.78-0.74 (m, 6H). | LC-MS (026): 490.10 [M + H]⁺; Rt = 2.01 min, purity 99%. |
| | 2-(2-((3,5-dimethylisoxazol-4- | | |
| | yl)(hydroxy)methyl)benzofuran- | | |
| | 5-yl)-N-(1-(3,5-dimethylpyridin- | | |
| | 2-yl)-4-methylpentyl)acetamide | | |
| 27 | 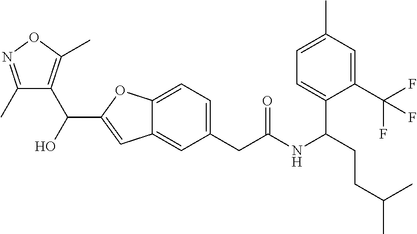 [SMILES: Cc1ccc(C(CCC(C)C)NC(=O)Cc2ccc3oc(C(O)c4c(C)noc4C)cc3c2)c(C(F)(F)F)c1] | ¹H NMR (DMSO, 400 MHz): 8.53 (d, J = 8 Hz, 1H), 7.56 (d, J = 8 Hz, 1H), 7.45-7.43 (m, 3H), 7.39 (s, 1H), 7.12 (d, J = 6.8 Hz, 1H), 6.71 (s, 1H), 6.18 (s, 1H), 5.81 (s, 1H), 5.03 (s, 1H), 3.54-3.43 (m, 2H), 2.33 (s, 6H), 2.10 (s, 3H), 1.56-1.53 (m, 2H), 1.45-1.41 (m, 1H), 1.10-1.04 (m, 1H), 0.85-0.83 (m, 1H), 0.79-0.74 (m, 6H). | LC-MS024: m/z 543.7 [M + H]⁺; Rt = 1.87 min, purity 100%. |
| | 2-(2-((3,5-dimethylisoxazol-4- | | |
| | yl)(hydroxy)methyl)benzofuran- | | |
| | 5-yl)-N-(4-methyl-1-(4-methyl- | | |
| | 2-(trifluoromethyl)phenyl) | | |
| | pentyl)acetamide | | |
| 28 | 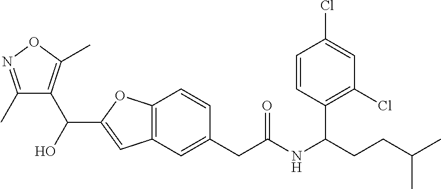 [SMILES: Cc1noc(C)c1C(O)c1cc2cc(CC(=O)NC(CCC(C)C)c3ccc(Cl)cc3Cl)ccc2o1] | ¹H NMR (DMSO, 400 MHz): δ 8.66 (d, J = 6 Hz, 1H), 7.54 (m, 5H), 7.16 (d, J = 6.4 Hz, 1H), 6.73 (s, 1H), 6.21 (s, 1H), 5.83 (s, 1H), 5.02 (s, 1H), 3.56 (m, 2H), 2.33 (s, 3H), 2.16 (s, 3H), 1.58-1.47 (m, 2H), 1.30- 1.12 (m, 3H), 0.81 (m, 6H). | LC-MS(022): m/z 529.0 [M + H]⁺; Rt = 1.90 min, purity 91.99%. |
| | N-(1-(2,4-dichlorophenyl)-4- | | |
| | methylpentyl)-2-(2-((3,5- | | |
| | dimethylisoxazol-4- | | |
| | yl)(hydroxy)methyl)benzofuran- | | |
| | 5-yl)acetamide | | |
| 29 | 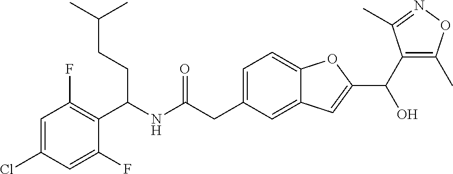 [SMILES: Cc1noc(C)c1C(O)c1cc2cc(CC(=O)NC(CCC(C)C)c3c(F)cc(Cl)cc3F)ccc2o1] | ¹H NMR (DMSO, 400 MHz): 8.60 (d, J = 7.2 Hz, 1H), 7.40 (d, J = 8.4 Hz, 2H), 7.28 (d, J = 7.6 Hz, 2H), 7.10 (d, J = 9.6 Hz, 1H), 6.72 (s, 1H), 6.19 (d, J = 4.4 Hz, 1H), 5.83 (d, J = 4.0 Hz, 1H), 4.97 (d, J = 8 Hz, 1H), 3.54-3.36 (m, 2H), 2.34 (s, 3H), 2.12 (s, 3H), 1.87-1.73 (m 2H), 1.51-1.47 (m, 1H), 1.24 (s, 1H), 0.99 (s, 1H), 0.83-0.79 (m, 6H). | LC-MS024: m/z 531.7, [M + H]⁺; Rt = 1.87 min, purity 100%. |
| | N-(1-(4-chloro-2,6- | | |
| | difluorophenyl)-4- | | |
| | methylpentyl)-2-(2-((3,5- | | |
| | dimethylisoxazol-4- | | |
| | yl)(hydroxy)methyl)benzofuran- | | |
| | 5-yl)acetamide | | |
| 30 | 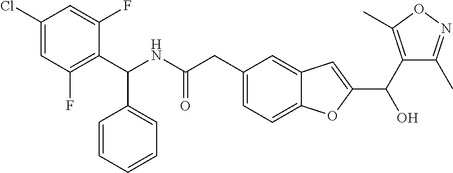 [SMILES: Cc1noc(C)c1C(O)c1cc2cc(CC(=O)NC(c3ccccc3)c3c(F)cc(Cl)cc3F)ccc2o1] | ¹H NMR (DMSO, 400 MHz): 9.01 (d, J = 8.0 Hz, 1H), 7.46-7.14 (m, 10H), 6.73 (s, 1H), 6.41 (d, J = 8 Hz, 1H), 6.18 (d, J = 4.4 Hz, 1H), 5.82 (t, J = 3.6 Hz, 1H), 3.68-3.60 (m, 2H), 2.33 (s, 3H), 2.11 (t, 3H). | LC-MS026: m/z 537.7, [M + H]⁺; Rt = 1.96 min, purity 100%. |
| | N-((4-chloro-2,6- | | |
| | difluorophenyl)(phenyl)methyl)- | | |
| | 2-(2-((3,5-dimethylisoxazol-4- | | |
| | yl)(hydroxy)methyl)benzofuran- | | |
| | 5-yl)acetamide | | |
| 31 | 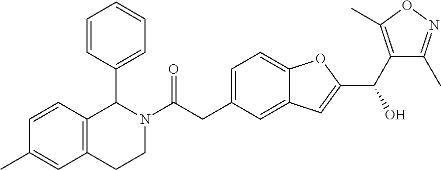 | ¹H NMR (MeOD, 400 MHz): δ 7.29- 6.79 (m, 11H), 6.69 (s, 1H),6.53 (s, 1H), 5.74 (s, 1H), 3.82-3.76 (m, 3H), 3.28-3.24 (m, 1H), 2.50-2.46 (m, 2H), 2.24 (s, 3H), 2.17 (s, 3H), 2.08 (s, 3H). | LC-MS (020): 507.2 [M + H]⁺; Rt: 1.70 min |
| | 2-(2-((S)-(3,5-dimethylisoxazol- | | |
| | 4-yl)(hydroxy)methyl) | | |
| | benzofuran-5-yl)-1-(6-methyl-1- | | |
| | phenyl-3,4-dihydroisoquinolin- | | |
| | 2(1H)-yl)ethanone | | |
| 32 | 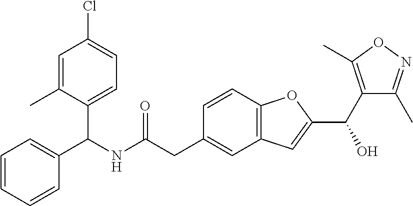 [SMILES: Cc1cc(Cl)ccc1C(NC(=O)Cc1ccc2oc([C@@H](O)c3c(C)noc3C)cc2c1)c1ccccc1] | ¹H NMR (400 MHz, CDCL₃) δ: 7.42 (m, J = 8.0 Hz, 2H), 7.26-7.23 (m, 2H), 7.19-7.13 (m, 2H), 7.07-7.99 (m, 3H), 6.82 (d, J = 8.0 Hz, 1H), 6.55 (s, 1H), 6.32 (d, J = 8.0 Hz, 1H), 5.88-5.85 (m, 2H), 3.71 (s, 2H), 2.39-2.37 (m, 4H), 2.26 (s, 3H), 2.18 (s, 3H). | LC-MS (044): 515.2 [M + H]⁺; Rt: 1.75 min, Purity: 99.41% (214). |
| | N-((4-chloro-2- | | |
| | methylphenyl)(phenyl)methyl)- | | |
| | 2-(2-((S)-(3,5-dimethylisoxazol- | | |
| | 4- | | |
| | yl)(hydroxy)methyl)benzofuran- | | |
| | 5-yl)acetamide | | |

Using essentially the same procedure as described in example 2, the following compounds in table 2 were made.

TABLE 2

| Ex. | Structure/Name | NMR | LCMS |
|---|---|---|---|
| 33 | 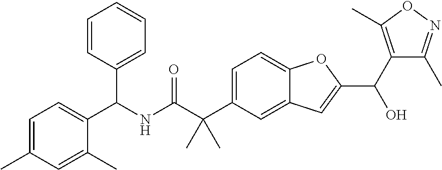 | ¹H NMR (400 MHz, DMSO-d₆) δ ppm 7.94 (d, J = 8.0 Hz, 1H), 7.49 (s, 1H), 7.42 (d, J = 8.4 Hz, 1H), 7.21-7.15 (m, 4H), 7.01 (d, J = 6.8 Hz, 2H), 6.92 (s, 1H), 6.81-6.72 (m, 3H), 6.24 (d, J = 8.0 Hz, 1H), 6.20 (d, J = 4.4 Hz, 1H), 5.81 (d, J = 4.0 Hz, 1H), 2.33 (s, 3H), 2.19 (s, 3H), 2.12 (s, 3H), 2.10 (s, 3H), 1.51 (s, 3H), 1.50 (s, 3H). | LCMS-P1: 523 [M + H]⁺; Rₜ = 1.791 min |
| | 2-(2-((3,5- | | |
| | dimethylisoxazol-4- | | |
| | yl)(hydroxy)methyl)benzo- | | |
| | furan-5-yl)-N-((2,4- | | |
| | dimethylphenyl)(phenyl) | | |
| | methyl)-2- | | |
| | methylpropanamide | | |
| 34 | 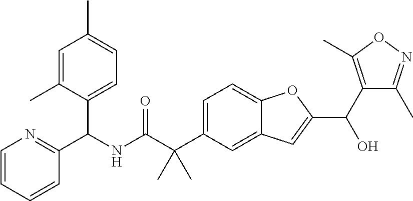 [SMILES: Cc1ccc(C(NC(=O)C(C)(C)c2ccc3oc(C(O)c4c(C)noc4C)cc3c2)c2ccccn2)c(C)c1] | ¹H NMR (400 MHz, DMSO-d₆,) δ ppm 8.40 (d, J = 3.6 Hz, 1H), 7.78-7.84 (m, 1H), 7.68 (t, J = 6.4 Hz, 1H), 7.52 (s, 1H), 7.43 (d, J = 8.4 Hz, 1H), 7.23-7.15 (m, 3H), 6.91 (s, 1H), 6.78 (d, J = 7.6 Hz, 1H), 6.73 (s, 1H), 6.63-6.61 (m, 1H), 6.21-6.20 (m, 2H), 5.83 (d, J = 3.6 Hz, 1H), 2.33 (s, 3H), 2.21 (s, 3H), 2.16 (s, 3H), 2.10 (s, 3H), 1.54 (s, 3H), 1.48 (s, 3H). | LCMS-P1: 524 [M + H]⁺; Rₜ = 1.584 min |
| | 2-(2-((3,5- | | |
| | dimethylisoxazol-4- | | |
| | yl)(hydroxy)methyl)benzo- | | |
| | furan-5-yl)-N-((2,4- | | |
| | dimethylphenyl)(pyridin-2- | | |
| | yl)methyl)-2- | | |
| | methylpropanamide | | |

### Example 35

### 2-(2-((3,5-dimethylisoxazol-4-yl)(hydroxy)methyl)benzofuran-5-yl)-N-(1-(2,4-dimethylphenyl)-2-isopropoxyethyl)acetamide

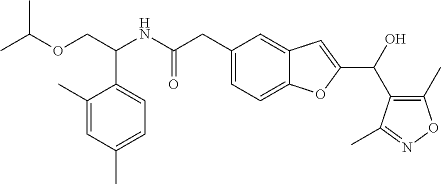 [SMILES: Cc1ccc(C(COC(C)C)NC(=O)Cc2ccc3oc(C(O)c4c(C)noc4C)cc3c2)c(C)c1]

### (a) 2-(chloromethoxy)propane

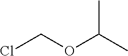

Dry hydrogen chloride was bubbled into a solution of (CH₂O)ₙ (11 g, 0.34 mol) in propan-2-ol (20 g, 0.34 mol) at 15-20° C. until all the solid was dissolved. The organic phase was separated and dried with CaCl₂. The title compound (15.6 g, yield: 49.6%) was purified by distillation. ¹H NMR (CDCl₃, 500 MHz): δ 5.54 (s, 2H), 4.10-4.00 (m, 1H), 1.23 (s, 3H), 1.22 (S, 3H).

### (b) 2-isopropoxyacetonitrile

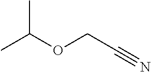

A well-stirred mixture of CuCN (2.31 g, 0.26 mol) in diethyl ether (30 mL) was heated to reflux and 2-(chloromethoxy)propane (10.8 g, 0.1 mol) in diethyl ether (30 mL) was added slowly over 1 h. The solid was removed by filtration. Title compound was obtained by distillation (3.3 g, yield: 34%). ¹H NMR (CDCl₃, 500 MHz): δ 4.24 (s, 2H), 3.86-3.77 (m, 1H), 1.23 (s, 3H), 1.21 (S, 3H).

### (c) 1-(2,4-dimethylphenyl)-2-isopropoxyethanamine

To a solution of 2-isopropoxyacetonitrile (500 mg, 0.005 mol) in THF (100 mL) was added (2,4-dimethylphenyl)magnesium bromide (5.225 g, 0.025 mol) at 0° C. under nitrogen. To the mixture was added MeOH (5 mL) followed by addition of NaBH₄ (0.028 g, 0.75 mol) at 0° C. To the mixture was added water (5 mL) and the mixture was extracted with EtOAc (10 mL×3). The organic layers were dried over Na₂SO₄, filtered, and concentrated under reduced pressure. Title compound was obtained by flash column (269 mg, yield: 26%). LCMS-A024: 208.7 [M+14]^{±}; Rt=1.30 min.

### (d) 2-(2-((3,5-dimethylisoxazol-4-yl)(hydroxy)methyl)benzofuran-5-yl)-N-(1-(2,4-dimethylphenyl)-2-isopropoxyethyl)acetamide

This compound was synthesized from 2-(2-((3,5-dimethylisoxazol-4-yl)(hydroxy)methyl)benzofuran-5-yl)acetic acid and 1-(2,4-dimethylphenyl)-2-isopropoxyethanamine essentially as described in example 1(k) to give title compound (40 mg, yield: 20.4%). LCMS-A024: 490.7 [M+H]⁺; Rt=1.72 min. ¹H NMR (DMSO-d₆, 400 MHz): δ 8.45 (d, J=8.0 Hz, 1H), 7.44-7.38 (m, 2H), 7.19-7.12 (m, 2H), 6.94-6.91 (m, 2H), 6.70 (s, 1H), 6.17 (s, 1H), 5.82-5.80 (d, J=8.0 Hz, 2H), 5.08-5.03 (m, 1H), 3.55-3.42 (m, 4H), 2.32 (s, 3H), 2.21 (d, J=8.0 Hz, 6H), 2.09 (s, 3H), 1.03-1.00 (m, 6H)

### Example 36

### 2-(2-((3,5-dimethylisoxazol-4-yl)(hydroxy)methyl)benzofuran-5-yl)-N-((4-hydroxy-2-methylphenyl)(phenyl)methyl)acetamide

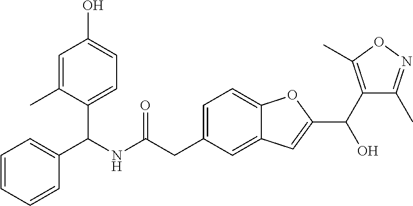 [SMILES: Cc1cc(O)ccc1C(NC(=O)Cc1ccc2oc(C(O)c3c(C)noc3C)cc2c1)c1ccccc1]

### (a) 4-hydroxy-2-methylbenzonitrile

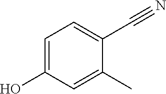

Decanethiol (261 mg, 1.5 mmol) and t-BuOK (168 mg, 1.5 mmol) were added to a solution of 4-methoxy-2-methylbenzonitrile (147 mg, 1 mmol) in DMF (5 mL). The reaction mixture was stirred at 110° C. for 3 h. The mixture was then diluted with water (30 mL) and extracted with EtOAc (10 mL×3). The extracts were washed with brine (10 mL×3), dried over Na₂SO₄, and concentrated under reduced pressure. The resulting residue was purified by silica gel column with (petroleum ether:EtOAc=10:1) to provide 4-hydroxy-2-methylbenzonitrile (70 mg, yield: 52.6%). LC-MS (011): 134.70 [M+H]⁺; Rt: 1.44 min, Purity: 80% (254 nm).

### (b) 4-(benzyloxy)-2-methylbenzonitrile

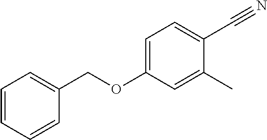 [SMILES: Cc1cc(OCc2ccccc2)ccc1C#N]

BnCl (806.5 mg, 4.69 mmol) and K₂CO₃ (1175.7 mg, 8.52 mmol) were added to a solution of 4-hydroxy-2-methylbenzonitrile (567 mg, 4.26 mmol) in acetonitrile (10 mL). The resulting mixture was stirred overnight at rt. The mixture was then diluted with water (30 mL) and extracted with EtOAc. The combined extracts were washed with brine (10 mL×3), dried over Na₂SO₄, and concentrated under reduced pressure to provide crude 4-(benzyloxy)-2-methylbenzonitrile (500 mg, yield: 52.6%), which used in the next step without further purification. LC-MS (026): 224.7 [M+H]+; Rt=1.63 min, purity 58%

### (c) (4-(benzyloxy)-2-methylphenyl)(phenyl)methanamine

This compound was synthesized from 4-(benzyloxy)-2-methylbenzonitrile and phenylmagnesium bromide essentially as described in example 1 (e) (400 mg, yield: 37%.). LC-MS (010): 288.7 [M−NH2]⁺; Rt=1.593 min, purity: 100% 214.

### (d) N-((4-(benzyloxy)-2-methylphenyl)(phenyl)methyl)-2-(2-((3,5-dimethylisoxazol-4-yl)(hydroxy)methyl)benzofuran-5-yl)acetamide

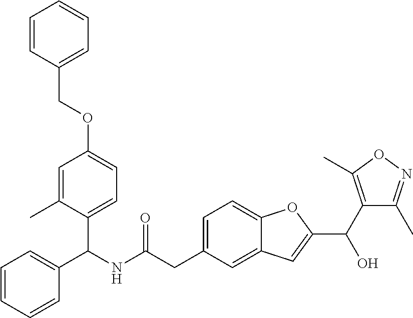 [SMILES: Cc1cc(OCc2ccccc2)ccc1C(NC(=O)Cc1ccc2oc(C(O)c3c(C)noc3C)cc2c1)c1ccccc1]

HOBt (62.3 mg, 0.462 mmol), EDCI (88.2 mg, 0.462 mmol), DIPEA (59.6 mg, 0.462 mmol) and 4-(benzyloxy)-2-methylphenyl)(phenyl)methanamine (70 mg, 0.231 mmol) were added to a solution of 2-(2-((3,5-dimethylisoxazol-4-yl)(hydroxy)methyl)benzofuran-5-yl)acetic acid (69.5 mg, 0.231 mmol) in CH₂Cl₂ (5 mL). The resulting mixture was stirred at rt overnight, then additional CH₂Cl₂ (5 mL) was added to the mixture. The mixture was then washed with water (10 mL×3), brine (10 mL×3), dried over Na₂SO₄, and concentrated under reduced pressure to yield a residue that was used directly in the next step without further purification. LC-MS (024): 569.7 [M−OH]+; Rt=1.538 min, purity 89%.

### (e) 2-(2-((3,5-dimethylisoxazol-4-yl)(hydroxy)methyl)benzofuran-5-yl)-N-((4-hydroxy-2-methylphenyl)(phenyl)methyl)acetamide

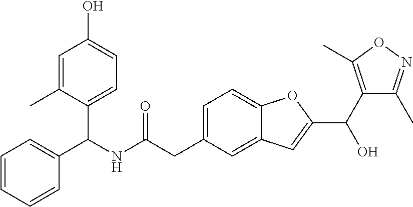 [SMILES: Cc1cc(O)ccc1C(NC(=O)Cc1ccc2oc(C(O)c3c(C)noc3C)cc2c1)c1ccccc1]

A mixture of N-((4-(benzyloxy)-2-methylphenyl)(phenyl)methyl)-2-(2-((3,5-dimethylisoxazol-4-yl)(hydroxy)methyl)benzofuran-5-yl)acetamide (300 mg, 0.512 mmol) and 10% Pd/C (30 mg) in methanol (8 mL) was stirred under hydrogen atmosphere (1 atm) for 3 h at rt. Then the catalyst was filtered off, the filtrate was concentrated to dryness to give crude product, which was purified by reverse phase HPLC using water/acetonitrile with 0.05% TFA to obtain title compound (120 mg, 40%). LCMS-A020): 479.70 [M−OH]⁺; Rt=1.93 min. 1H NMR (DMSO-d6, 400 MHz): δ 9.22 (s, 1H), 8.79 (d, J=8.4 Hz, 1H), 7.47 (s, 1H), 7.43 (d, J=8.8 Hz, 1H), 7.31-7.28 (m, 2H), 7.23 (d, J=7.2 Hz, 1H), 7.16 (d, J=6.8 Hz, 3H), 6.82 (d, J=8.4 Hz, 1H), 6.74 (s, 1H), 6.57 (s, 1H), 6.52 (d, J=8.4 Hz, 1H), 6.18 (d, J=4.8 Hz, 1H), 6.12 (d, J=8 Hz, 1H), 5.83 (d, J=4.4 Hz, 1H), 3.57 (br, 2H), 2.34 (br, 3H), 2.12 (br, 3H), 2.11 (br, 3H).

### Example 37

### 2-(2-((3,5-dimethylisoxazol-4-yl)(hydroxy)methyl)benzofuran-5-yl)-N-((2,4-dimethylphenyl)(4-(methylsulfonamido)phenyl)methyl)acetamide

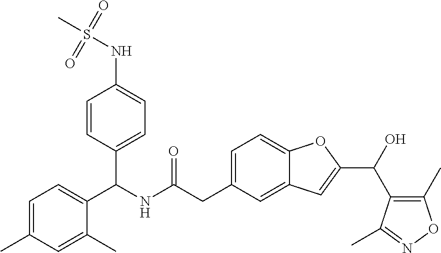

### (a) N-(3-((2,4-dimethylphenyl)(hydroxy)methyl)phenyl)methanesulfonamide

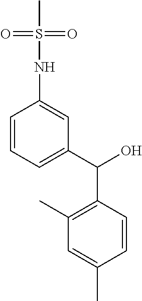 [SMILES: Cc1ccc(C(O)c2cccc(NS(C)(=O)=O)c2)c(C)c1]

n-Butyllithium solution (1.6 in hexanes, 2.2 mL, 3.52 mmol) was added drop-wise to a solution of 1-bromo-2,4-dimethylbenzene (542.3 mg, 2.93 mmol) in THF (15 mL) at −78° C. The mixture was then stirred for 1 h at −78° C. Then N-(3-formylphenyl)methanesulfonamide was injected to the mixture and the mixture was allowed to warm up to rt slowly and stirred at rt overnight. THF/H₂O (10 mL/2 mL) was then added to the mixture. The mixture was then extracted with EtOAc (3×40 mL). The combined extracts were concentrated under reduced pressure and purified by gel-silica-column (petroleum ether:EtOAc=4:1) to get the title compound (536 mg, 60%). LCMS-A024: 328.7 [M+Na]⁺; Rt: 1.267 min.

### (b) N-(3-(azido(2,4-dimethylphenyl)methyl)phenyl)methanesulfonamide

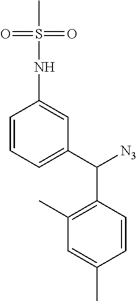 [SMILES: Cc1ccc(C(N=[N+]=[N-])c2cccc(NS(C)(=O)=O)c2)c(C)c1]

TFA (1 mL) was added to a solution of N-(3-((2,4-dimethylphenyl)(hydroxy)methyl)phenyl) methanesulfonamide (200 mg, 0.65 mmol) in CH₂Cl₂ (7 mL), followed by addition of NaN₃ (128 mg 1.96 mmol). Then the mixture was stirred at rt overnight. NaHCO₃ (sat.) solution (5 mL) was added and the pH of reaction mixture was adjusted to ˜7. The mixture was extracted with EtOAc (3×20 mL). The organic solvent was collected and removed under reduced pressure. The residue was purified by silica gel Prep-TLC to obtained title compound (130 mg, yield 59.1%).

### (c) N-(3-(amino(2,4-dimethylphenyl)methyl)phenyl)methanesulfonamide

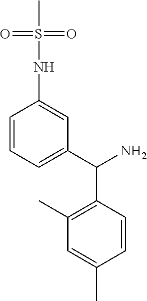 [SMILES: Cc1ccc(C(N)c2cccc(NS(C)(=O)=O)c2)c(C)c1]

PPh₃ (113.5 mg, 0.433 mmol) was added to a solution of N-(3-(azido(2,4-dimethylphenyl)methyl)phenyl)methanesulfonamide (130 mg, 0.394 mmol) in THF (6 mL) under nitrogen. The reaction mixture was stirred at 30° C. overnight, then water (10.6 mg, 0.591 mmol) was added. After stirring at rt for 2 h, the reaction mixture was extracted with ethyl acetate (3×20 mL), and the combined organic layers were dried over anhydrous sodium sulfate, and concentrated under reduced pressure. The resulting residue was purified by silica gel Prep-TLC (petroleum ether:EtOAc=2:1) to get title compound (43 mg, yield 40%). LCMS-A024: 288.7 [M−NH₂]; Rt: 1.33 min

### (d) 2-(2-((3,5-dimethylisoxazol-4-yl)(hydroxy)methyl)benzofuran-5-yl)-N-((2,4-dimethylphenyl)(4-(methylsulfonamido)phenyl)methyl)acetamide

This compound was synthesized from 2-(2-((3,5-dimethylisoxazol-4-yl)(hydroxy)methyl)benzofuran-5-yl)acetic acid and N-(3-(amino(2,4-dimethylphenyl)methyl)phenyl)methanesulfonamide essentially as described in example 1(k) to give the title compound (20 mg, 24%). LCMS-A024: 570.7 [M−OH]; Rt: 1.60 min. ¹H NMR (CDCl₃, 400 MHz): δ 7.49 (s, 1H), 7.40 (d, J=8.4 Hz, 1H), 7.22-7.18 (m, 2H), 7.08 (d, J=8 Hz, 1H), 6.99 (s, 1H), 6.90-6.79 (m, 3H), 6.70-6.66 (m, 1H), 6.60-6.55 (m, 2H), 6.34 (d, J=8.4 Hz, 1H), 6.05-5.99 (m, 1H), 5.87 (s, 1H), 3.77-3.65 (m, 2H), 2.89 (s, 3H), 2.39 (s, 3H), 2.28 (s, 3H), 2.25 (s, 6H).

### Example 38

### N-((2-chloro-4-methylphenyl)(phenyl)methyl)-2-(2-(hydroxy(pyridin-4-yl)methyl)benzofuran-5-yl)acetamide

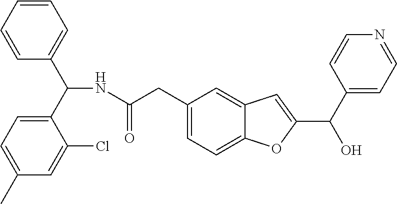 [SMILES: Cc1ccc(C(NC(=O)Cc2ccc3oc(C(O)c4ccncc4)cc3c2)c2ccccc2)c(Cl)c1]

### (a) 2-chloro-4-methylbenzonitrile

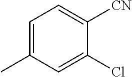

A solution of 1-bromo-2-chloro-4-methylbenzene (1.5 g, 7.4 mmol) and CuCN (1.3 g, 14.7 mmol) in DMF (20 mL) was heated to 150° C. for 5 h. After cooling to rt, the solid was removed by filtration. The filtrate was extracted with EtOAc (50 mL×3), the combined extracts were washed with brine (10 mL), dried over Na₂SO₄, and concentrated under reduced pressure. The resulting residue was purified by flash column (petroleum ether/EtOAc=20/1) to yield 2-chloro-4-methylbenzonitrile as a white solid (800 mg, yield: 72%). LCMSA026: No mass signal, Rt: 1.747 min, Purity 100%.

### (b) (2-chloro-4-methylphenyl)(phenyl)methanamine

This compound was synthesized from 2-chloro-4-methylbenzonitrile and phenylmagnesium bromide essentially as described in example 1 (e) (200 mg. yield 38%). LCMSA026: 232.0, Rt: 1.813 min, Purity 78%.

### (c) methyl 2-(2-isonicotinoylbenzofuran-5-yl)acetate

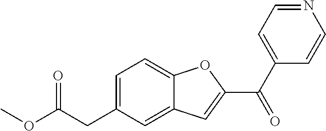 [SMILES: COC(=O)Cc1ccc2oc(C(=O)c3ccncc3)cc2c1]

K₂CO₃ (15.18 g, 110 mmol) was added to a solution of methyl 2-(3-formyl-4-hydroxyphenyl)acetate (9.7 g, 50 mmol) in DMF (160 mL) at rt. After stirring at rt for 30 min, 2-bromo-1-(pyridin-4-yl)ethanone hydrobromide (14.05 g, 50 mmol) was added to the mixture. The mixture was stirred at rt overnight, and water was added. The reaction mixture was extracted with EtOAc (250 mL×3), and the combined extracts were dried over Na₂SO₄, and concentrated under reduced pressure. The resulting crude product was purified by flash column (petroleum ether/EtOAc=1/1 to 1/1) to give 3.9 grams of title compound (yield: 26%). LCMSA024: 296.1 [M+H]+; Rt: 1.237 min

### (d) 2-(2-isonicotinoylbenzofuran-5-yl)acetic acid

This compound was synthesized from methyl 2-(2-isonicotinoylbenzofuran-5-yl)acetate essentially as described in example 1 (j) (650 mg, 97.6%). LCMS-A027: 282.3 [M+H]+; Rt=1.10 min

### (e) N-((2-chloro-4-methylphenyl)(phenyl)methyl)-2-(2-isonicotinoylbenzofuran-5-yl)acetamide

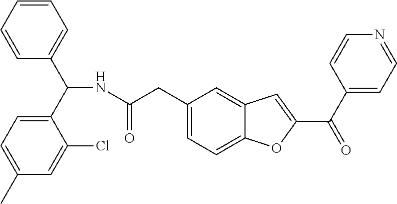 [SMILES: Cc1ccc(C(NC(=O)Cc2ccc3oc(C(=O)c4ccncc4)cc3c2)c2ccccc2)c(Cl)c1]

HOBt (28 mg, 0.21 mmol), EDCI (40 mg, 0.21 mmol), DIPEA (46 mg, 0.21 mmol) and (2-chloro-4-methylphenyl)(phenyl)methanamine (49 mg, 0.21 mmol) were added to a solution of 2-(2-isonicotinoylbenzofuran-5-yl)acetic acid (50 mg, 0.18 mmol) in DMF (4 mL). The resulting mixture was stirred at rt overnight. Water (10 mL) was added to the mixture and the mixture was extracted with ethyl acetate (20 mL×3). The combined extracts were washed with brine (15 mL), LiCl solution (15 mL×3) and dried over Na₂SO₄. Removal of solvent under reduced pressure yielded crude product (80 mg), which was used in the next step without further purification. LCMSA024: 495.1 [M+H]⁺; Rt: 1.454 min.

### (e) N-((2-chloro-4-methylphenyl)(phenyl)methyl)-2-(2-(hydroxy(pyridin-4-yl)methyl)benzofuran-5-yl)acetamide

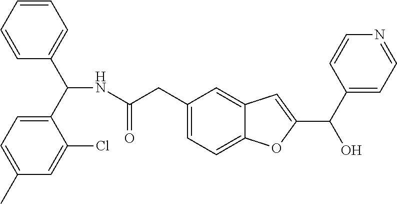 [SMILES: Cc1ccc(C(NC(=O)Cc2ccc3oc(C(O)c4ccncc4)cc3c2)c2ccccc2)c(Cl)c1]

NaBH₄ (12 mg, 0.32 mmol) was added to a stirred solution of N-((2-chloro-4-methylphenyl)(phenyl)methyl)-2-(2-isonicotinoylbenzofuran-5-yl)acetamide (80 mg, 0.16 mmol) in MeOH (10 mL). After stirring at rt for 2 h, the mixture was extracted with EtOAc (15 mL×3). The combined extracts were washed with brine (10 mL), dried over Na₂SO₄, and concentrated under reduced pressure. The resulting crude product was purified by flash column (petroleum ether/EtOAc=1/1 to CH2Cl2/EtOAc=4/1) to yield N-((2-chloro-4-methylphenyl)(phenyl)methyl)-2-(2-(hydroxy(pyridin-4-yl)methyl)benzofuran-5-yl)acetamideas white solid (36 mg, yield 48%). LCMSA022: 497.1 [M+H]⁺; Rt: 1.286 min., Purity 93%. ¹H NMR (CDCl₃, 400 MHz): δ 8.62 (d, J=5.6 Hz, 2H), 7.47-7.39 (m, 4H), 7.24-7.15 (m, 5H), 7.05-6.98 (m, 4H), 6.56 (s, 1H), 6.46 (d, J=8.0 Hz, 1H), 6.17 (d, J=8.0 Hz, 1H), 5.95 (s, 1H), 3.71 (s, 2H), 2.30 (s, 3H).

### Example 39

### 2-(2-((3,5-dimethylisoxazol-4-yl)(hydroxy)methyl)benzofuran-5-yl)-N-(1-(2,6-dimethylpyridin-3-yl)-4-methylpentyl)acetamide

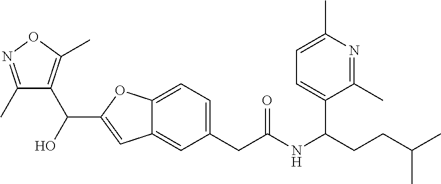 [SMILES: Cc1ccc(C(CCC(C)C)NC(=O)Cc2ccc3oc(C(O)c4c(C)noc4C)cc3c2)c(C)n1]

### (a) 2,6-dimethylnicotinonitrile

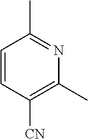

A mixture of 3-bromo-2,6-dimethylpyridine (1.1 g, 5.79 mmol) and CuCN (1.31 mg, 14.5 mmol) in DMF (40 ml) was stirred under nitrogen and refluxed overnight. The reaction mixture was then cooled to rt and filtered. The filtrate was diluted with water (300 ml) and extracted with EtOAc (200 ml×2). The combined extracts were dried over Na₂SO₄ and concentrated under reduced pressure to give the crude product. The residue was purified by silica-gel-column (petroleum ether:EtOAc=20:1) to obtain 2,6-dimethylnicotinonitrile (400 mg, 50.6%) as yellow solid. LC-MS (CP-0006802-035-00459): 133.1 [M+H]+; Rt: 1.19 min.

### (b) 1-(2,6-dimethylpyridin-3-yl)-4-methylpentan-1-amine

This compound was synthesized from 2,6-dimethylnicotinonitrile and isopentylmagnesium bromide essentially as described in example 1 (e) (500 mg, 6.2%). LC-MS (CP-0006802-104-00450): 207.1 [M+H]+; Rt: 1.26 min

### (c) 2-(2-((3,5-dimethylisoxazol-4-yl)(hydroxy)methyl)benzofuran-5-yl)-N-(1-(2,6-dimethylpyridin-3-yl)-4-methylpentyl)acetamide

This compound was synthesized from 1-(2,6-dimethylpyridin-3-yl)-4-methylpentan-1-amine and 2-(2-((3,5-dimethylisoxazol-4-yl)(hydroxy)methyl)benzofuran-5-yl)acetic acid essentially as described in example 1 (k) (8.4 mg, Yield: 15%). LC-MS (022): m/z 490.1, [M+H]⁺; Rt=1.27 min, purity 91.8%. ¹H NMR (DMSO, 400 MHz): δ 8.53 (d, J=6.4 Hz, 1H), 7.53 (d, J=6.4 Hz, 1H), 7.44 (s, 1H), 7.41 (d, J=8.0 Hz, 1H), 7.14 (d, J=6.8 Hz, 1H), 7.04 (d, J=6.4 Hz, 1H), 6.72 (s, 1H), 6.24 (d, J=3.6 Hz, 1H), 5.83 (d, J=3.2 Hz, 1H), 4.85-4.80 (m, 1H), 2.41 (s, 3H), 2.36 (s, 3H), 2.33 (s, 3H), 2.11 (s, 3H), 1.60 (m, 2H), 1.46 (m, 1H), 1.25 (m, 2H), 0.86 (m, 6H).

### Example 40

N-(1-(2-(dimethylamino)-4-methylphenyl)-4-methylpentyl)-2-(2-((3,5-dimethylisoxazol-4-yl)(hydroxy)methyl)benzofuran-5-yl)acetamide

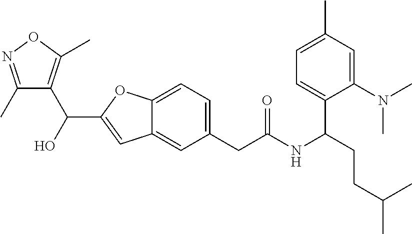 [SMILES: Cc1ccc(C(CCC(C)C)NC(=O)Cc2ccc3oc(C(O)c4c(C)noc4C)cc3c2)c(N(C)C)c1]

### (a) 2-(dimethylamino)-4-methylbenzonitrile

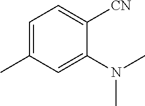

A mixture of 2-amino-4-methylbenzonitrile (400 mg, 3 mmol), HCHO (37% aq, 4.91 g, 60 mmol), AcOH (0.5 ml) and NaCNBH3 (378 mg, 9 mmol) in CH3CN (20 ml) was stirred at rt overnight. The mixture was then concentrated under reduced pressure, diluted with EtOAc (30 ml), washed with brine (40 ml), dried over sodium sulfate, filtered, concentrated under reduced pressure, and purified by Pre-TLC (Petroleum ether:EtOAc=7:1) to afford 2-(dimethylamino)-4-methylbenzonitrile (230 mg, 47%) as yellow oil. LC-MSA027: CP-0008579-060-01529-LCMSA027, m/z 161 [M+H]+; Rt=1.11 min. Purity 93% (214 nm).

### (b) 2-(1-amino-4-methylpentyl)-N,N,5-trimethylaniline

This compound was synthesized from 2-(dimethylamino)-4-methylbenzonitrile and isopentylmagnesium bromide essentially as described in example 1 (e) (120 mg, Yield: 56.8%). LC-MS: CP-0006802-142-01824, 234 [M+H]+; Rt=1.32 min, Purity 97% (214 nm).

### (c) N-(1-(2-(dimethylamino)-4-methylphenyl)-4-methylpentyl)-2-(2-((3,5-dimethylisoxazol-4-yl)(hydroxy)methyl)benzofuran-5-yl)acetamide

This compound was synthesized from 2-(1-amino-4-methylpentyl)-N,N,5-trimethylaniline and 2-(2-((3,5-dimethylisoxazol-4-yl)(hydroxy)methyl)benzofuran-5-yl)acetic acid essentially as described in example 1 (k) (20 mg, Yield: 40.0%). LC-MS024: m/z 518.7 [M+H]⁺; Rt=1.36 min, purity 91.8%. ¹H NMR (MeOD, 400 MHz): δ 7.50 (s, 1H), 7.37 (d, J=8 Hz, 1H), 7.22 (d, J=1.6 Hz, 1H), 7.12 (d, J=8 Hz, 1H), 7.00 (s, 1H), 6.88 (s, 1H), 6.70 (s, 1H), 5.88 (s, 1H), 5.33 (s, 1H), 3.61 (s, 2H), 2.57 (s, 6H), 2.37 (s, 3H), 2.28 (s, 3H), 2.20 (s, 3H), 1.69-1.66 (m, 2H), 1.52-1.48 (m, 1H), 1.18-1.06 (m, 2H), 0.84-0.80 (m, 6H).

### Example 41, 42, 42, 44

### 2-(2-((S)-(3,5-dimethylisoxazol-4-yl)(hydroxy)methyl)benzofuran-5-yl)-N-((S)-1-(3,5-dimethylpyridin-2-yl)-4-methylpentyl)acetamide, 2-(2-((S)-(3,5-dimethylisoxazol-4-yl)(hydroxy)methyl)benzofuran-5-yl)-N-((R)-1-(3,5-dimethylpyridin-2-yl)-4-methylpentyl)acetamide, 2-(2-((R)-(3,5-dimethylisoxazol-4-yl)(hydroxy)methyl)benzofuran-5-yl)-N-((R)-1-(3,5-dimethylpyridin-2-yl)-4-methylpentyl)acetamide, and 2-(2-((R)-(3,5-dimethylisoxazol-4-yl)(hydroxy)methyl)benzofuran-5-yl)-N-((S)-1-(3,5-dimethylpyridin-2-yl)-4-methylpentyl)acetamide

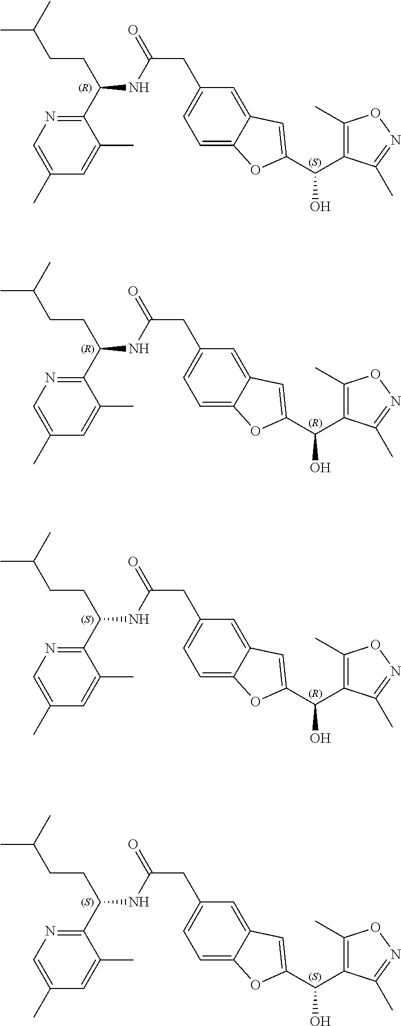

2-(2-((3,5-dimethylisoxazol-4-yl)(hydroxy)methyl)benzofuran-5-yl)-N-(1-(3,5-dimethylpyridin-2-yl)-4-methylpentyl)acetamide (650 mg) was resolved using the following method:

Instrument: Thar SFC Prep 200 (Thar Technologies, Waters); Column: ChiralPak AD-H, 50 mm I.D.×250 mm Length, 5 μm (Daicel Chemical Industries Co., Ltd); Column Temperature: 35° C. Mobile Phase: CO2/EtOH/DEA=70/30/0.1; Flow rate: 160 g/min; Back Pressure: 100 Bar; Wavelength: 214 nm; Cycle time: 12.1 min; Injection Volume: 3.0 mL; Load per injection: 87.5 mg; Feed solution: 700 mg dissolved in 24 mL MeOH
Said resolution yielded 4 diastereomers:
peak 1, 75 mg, RT: 5.8 min, 100% de, 100% purity
peak 2, 80 mg, RT: 6.9 min, 96.86% de, 98.43% purity
peak 3, 69 mg, RT: 8.3 min, 100% de, 100% purity
peak 4, 73 mg, RT: 14.2 min, 100% de, 100% purity

### Example 45

### 2-(2-((3,5-dimethylisoxazol-4-yl)(hydroxy)methyl)benzofuran-5-yl)-N-(1-(3,5-dimethylpyridin-2-yl)-1-deutero-4-methylpentyl)acetamide

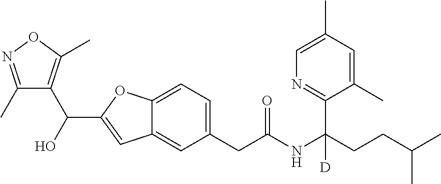 [SMILES: [2H]C(CCC(C)C)(NC(=O)Cc1ccc2oc(C(O)c3c(C)noc3C)cc2c1)c1ncc(C)cc1C]

### (a) 1-(3,5-dimethylpyridin-2-yl)-1-deutero-4-methylpentan-1-amine

This compound was synthesized from 4-(benzyloxy)-2-methylbenzonitrile and phenylmagnesium bromide essentially as described in example 1 (e) but using sodium borodeuteride instead of sodium borohydride (120 mg, yield 58%). LCMSA024: 208.3 [M+H]⁺; Rt: 1.293 min.

### (b) 2-(2-((3,5-dimethylisoxazol-4-yl)(hydroxy)methyl)benzofuran-5-yl)-N-(1-(3,5-dimethylpyridin-2-yl)-1-fluoro-4-methylpentyl)acetamide

This compound was synthesized from 1-(3,5-dimethylpyridin-2-yl)-1-deutero-4-methylpentan-1-amine and 2-(2-((3,5-dimethylisoxazol-4-yl)(hydroxy)methyl)benzofuran-5-yl)acetic acid essentially as described in example 1 (k) (65 mg, yield: 22%) as a white solid. LCMSA024: 491.3 [M+H]⁺; Rt: 1.399 min. ¹H NMR (CDCl₃, 400 MHz): δ 8.10 (s, 1H), 7.44 (s, 1H), 7.38 (d, J=8.4 Hz, 1H), 7.28 (s, 1H), 7.19 (dd, J=2.0 Hz, 8.8 Hz, 1H), 6.91 (br, 1H), 6.53 (s, 1H), 5.87 (s, 1H), 3.68-3.60 (m, 2H), 2.59 (s, 1H), 2.39 (s, 3H), 2.34 (s, 3H), 2.27 (s, 3H), 2.26 (s, 3H), 1.73-1.65 (m, 2H), 1.47-1.39 (m, 2H), 1.08-1.02 (m, 1H), 0.77 (s, 3H), 0.76 (s, 3H).

### Example 46

### 2-(2-((3,5-dimethylisoxazol-4-yl)(hydroxy)methyl)benzofuran-5-yl)-N-((2,5-dimethyloxazol-4-yl)(phenyl)methyl)acetamide

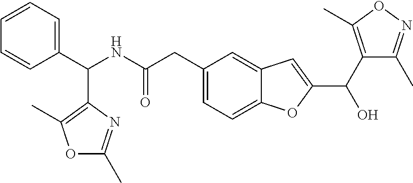 [SMILES: Cc1nc(C(NC(=O)Cc2ccc3oc(C(O)c4c(C)noc4C)cc3c2)c2ccccc2)c(C)o1]

### (a) (2,5-dimethyloxazol-4-yl)(phenyl)methanol

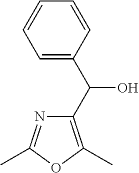

PhMgBr (2.4 mL, 1N in THF, 2.4 mmol) was added to a solution of 2,5-dimethyloxazole-4-carbaldehyde (150 mg, 1.2 mmol) in THF (6 mL) at 0° C. under N₂ atmosphere. After stirring at 0° C. for 3 h, the mixture was quenched with NH₄Cl (sat., 2 mL) and extracted with EtOAc (25 mL×3). The combined extracts were dried over Na₂SO₄ and concentrated under reduced pressure to yield a residue that was purified by flash column (petroleum ether/EtOAc=4/1) resulting in (2,5-dimethyloxazol-4-yl)(phenyl)methanol (115 mg, 47% yield) as an amber oil. LCMSA024: 204.1 [M+H]⁺; Rt=1.381 min; Purity: 100% (214 nm).

### (b) 4-(azido(phenyl)methyl)-2,5-dimethyloxazole

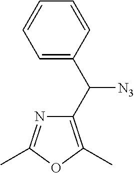 [SMILES: Cc1nc(C(N=[N+]=[N-])c2ccccc2)c(C)o1]

NaN₃ (52 mg, 0.8 mmol) was added to a solution of (2,5-dimethyloxazol-4-yl)(phenyl)methanol (80 mg, 0.4 mmol) in CH₂Cl₂ (6 mL), followed by addition of TFA (1 mL). The reaction mixture was stirred at rt overnight, and extracted with CH₂Cl₂ (15 mL×3). The combined extracts were washed with NaHCO₃ (10 mL×2), dried over Na₂SO₄, and concentrated under reduced pressure to yield 4-(azido(phenyl)methyl)-2,5-dimethyloxazole (86 mg, yield: 94%), which was used directly without and further purification. LCMSA024: 204.1 [M+H]⁺; Rt=1.713 min; Purity: 88% (214 nm).

### (c) (2,5-dimethyloxazol-4-yl)(phenyl)methanamine

This compound was synthesized from 4-(azido(phenyl)methyl)-2,5-dimethyloxazole essentially as described in example 37 (c) (90 mg, yield: 100%). LCMSA022: 186.2 [M−NH₂]^{±}; Rt=1.164 min; Purity: 92.7%(214 nm).

### (d) 2-(2-((3,5-dimethylisoxazol-4-yl)(hydroxy)methyl)benzofuran-5-yl)-N—((2,5-dimethyloxazol-4-yl)(phenyl)methyl)acetamide

This compound was synthesized from (2,5-dimethyloxazol-4-yl)(phenyl)methanamine and 2-(2-((3,5-dimethylisoxazol-4-yl)(hydroxy)methyl)benzofuran-5-yl)acetic acid essentially as described in example 1 (k) (4 mg, yield: 6%). LCMSA024: 486.2 [M+H]⁺; Rt=1.562 min; Purity=100%(214 nm, 254 nm). ¹H NMR (CDCl₃, 400 MHz): δ 7.45 (s, 1H), 7.38 (d, J=8.0 Hz, 1H), 7.34 (d, J=8.4 Hz, 1H), 7.25-7.24 (m, 2H), 7.22-7.21 (m, 1H), 7.19-7.16 (m, 2H), 6.51 (s, 1H), 6.16 (d, J=8.4 Hz, 1H), 5.84 (d, J=3.6 Hz, 1H), 3.69 (s, 2H), 2.41 (s, 3H), 2.38 (s, 3H), 2.32 (s, 3H), 2.24 (s, 3H).

### Example 47

### N-(1-(3,5-dimethylpyridin-2-yl)-4-methylpentyl)-2-(2-(hydroxy(pyridin-4-yl)methyl)benzofuran-5-yl)acetamide

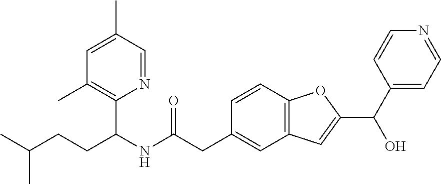 [SMILES: Cc1cnc(C(CCC(C)C)NC(=O)Cc2ccc3oc(C(O)c4ccncc4)cc3c2)c(C)c1]

### (a) Methyl 2-(2-(hydroxy(pyridin-4-yl)methyl)benzofuran-5-yl)acetate

i-PrMgCl (2.25 mL, 4.5 mmol) was added to a solution of methyl 2-(2-bromobenzofuran-5-yl)acetate (804 mg, 3 mmol) in 10 mL THF at 0° C. The mixture was stirred at 0° C. for 30 min. Then isonicotinaldehyde (482 mg, 4.5 mmol) was added to the mixture, the resulting mixture was stirred for 2 h. A saturated aqueous solution of NH₄Cl (20 mL) was added to the mixture, and the mixture was extracted with EtOAc (20 mL×3). The combined organic layers were washed with brine (20 mL), dried over Na₂SO₄, and concentrated under reduced pressure. The resulting residue was purified by silica gel column chromatography (30% EtOAc/petroleum ether) to afford methyl 2-(2-(hydroxy(pyridin-4-yl)methyl)benzofuran-5-yl)acetate (430 mg, yield 48.3%). LCMS A024: m/z 298.7 [M+H]⁺; Rt=1.14 min, purity 67%.

### (b) 2-(2-(Hydroxy(pyridin-4-yl)methyl)benzofuran-5-yl)acetic acid

NaOH (120 mg, 3 mmol) was added to a solution of methyl 2-(2-(hydroxy(pyridin-4-yl)methyl)benzofuran-5-yl)acetate (350 mg, 1.18 mmol) in 3 mL of MeOH and 1 mL of H₂O. After stirring at rt for 8 h, the aqueous phase was acidified to pH˜5 with con. HCl. The solution was extracted with EtOAc (3×10 mL). The combined extracts were dried over anhydrous sodium sulfate and filtered. The filtrate was concentrated to dryness under reduced pressure to obtain desired 2-(2-(hydroxy(pyridin-4-yl)methyl)benzofuran-5-yl)acetic acid as a ˜1:1 mixture with 2-(2-isonicotinoylbenzofuran-5-yl)acetic acid (300 mg, yield: 90%). 2-(2-(hydroxy(pyridin-4-yl)methyl)benzofuran-5-yl)acetic acid LCMS A024: m/z 284.7 [M+H]⁺; Rt=1.05 min, purity 51.36%; 2-(2-isonicotinoylbenzofuran-5-yl)acetic acid LCMS A024: m/z 282.7 [M+H]⁺; Rt=1.23 min, purity 48.64%.

### (c) 1-(3,5-dimethylpyridin-2-yl)-4-methylpentan-1-amine

To a solution of 3,5-dimethylpicolinonitrile (792 mg, 6 mmol) in 20 mL of THF under N₂ protection at 0° C. was dropped isopentylmagnesium bromide which was freshly prepared and used immediately (0.5N in THF, 21.6 mL). After the addition, the mixture was allowed to warm up to rt and stirred for 2 h. Then NaBH₄ (456 mg, 12 mmol) was added followed by addition of MeOH (15 mL). After stirring for 3 h, 15 mL of NH₄Cl (sat. 15 mL) aqueous solution was added to quench the reaction, and the mixture was extracted with EtOAc (20 mL×4). The combined EtOAc extracts were extracted with 1N HCl (15 mL×3). The pH value of the aqueous phase was adjusted to ˜11, and then EtOAc (20 mL×3) was used to extract the desired amine. The combined EtOAc extracts were dried over Na₂SO₄. Removal of solvent under reduced pressure yielded crude 1-(3,5-dimethylpyridin-2-yl)-4-methylpentan-1-amine (800 mg) as an amber oil, which was used directly with any further purification. LCMSA024: 207.2 [M+H]⁺; Rt=1.29 min; Purity=100% (254 nm).

### (d) N-(1-(3,5-dimethylpyridin-2-yl)-4-methylpentyl)-2-(2-(hydroxy(pyridin-4-yl)methyl)benzofuran-5-yl)acetamide

This compound was synthesized from 1-(3,5-dimethylpyridin-2-yl)-4-methylpentan-1-amine and 2-(2-(hydroxy(pyridin-4-yl)methyl)benzofuran-5-yl)acetic acid essentially as described in example 1 (k) (20 mg, yield: 27.8%). LC-MS A012 m/z 472.7 [M+H]⁺; Rₜ=1.40 min, purity 100%. 1H NMR (CDCL3, 400 MHz): δ 8.83-8.77 (m, 1H), 8.08 (s, 1H), 7.52-7.48 (m, 2H), 7.37 (d, J=8.4 Hz, 1H), 7.27-7.26 (m, 4H), 7.19-7.16 (m, 1H), 6.93 (s, 1H), 6.52 (s, 1H), 5.92 (s, 1H), 5.25-5.20 (m, 1H), 3.63 (d, J=2 Hz, 2H), 2.34 (s, 3H), 2.26 (s, 3H), 1.45-1.38 (m, 2H), 1.33-1.25 (m, 1H), 1.07-1.02 (m, 2H), 0.76 (d, J=6.8 Hz, 6H).

### Example 48

### 2-(2-((3,5-dimethylisoxazol-4-yl)(hydroxy)methyl)benzofuran-5-yl)-N-(1-(2,5-dimethyloxazol-4-yl)-4-methylpentyl)acetamide

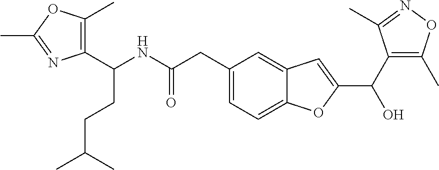

### (a) 1-(2,5-dimethyloxazol-4-yl)-4-methylpentan-1-ol

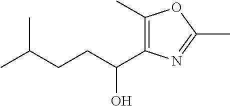

1-Bromo-3-methylbutane (400 mg, 2.64 mmol) and a catalytic amount of I₂ were added to a suspension of Mg (192 mg, 8 mmol) in THF (10 mL). The reaction was initiated by heating, and the remainder of 1-bromo-3-methylbutane (500 mg, 3.31 mmol) was added drop-wise. The mixture was stirred at rt for 4 h under nitrogen until most of Mg was consumed. 2,5-Dimethyloxazole-4-carbaldehyde (500 g, 4 mmol) was then added drop-wise, and the reaction mixture was stirred at rt for 16 h. NH₄Cl solution (10 mL) was then added and the mixture was extracted with EtOAc (10 mL×3). The combined extracts were dried over Na₂SO₄ and concentrated under reduced pressure to provide crude 1-(2,5-dimethyloxazol-4-yl)-4-methylpentan-1-ol (600 mg yield: 76.5%), which was used in the next step without further purification. LC-MS (022): 197.7 [M+H]⁺; Rt=1.340 min. Purity: 70% (214 nm)

### (b) 1-(2,5-dimethyloxazol-4-yl)-4-methylpentan-1-one

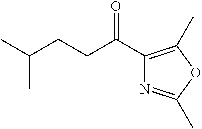

MnO₂ was added to a solution of 1-(2,5-dimethyloxazol-4-yl)-4-methylpentan-1-ol (600 mg, 3.04 mmol) dissolved in CH₂Cl₂ (6 mL), then the mixture was refluxed overnight. The precipitate that formed was filtered off and the filtrate was concentrated under reduced pressure to get crude product, which was purified by flash column (petroleum ether:EtOAc=20:1) to give 1-(2,5-dimethyloxazol-4-yl)-4-methylpentan-1-one (423 mg, yield: 71.2%). LC-MS (011): 196.7 [M+H]⁺; Rt=1.830 min. Purity: 90.5% (254 nm)

### (c) 1-(2,5-dimethyloxazol-4-yl)-4-methylpentan-1-one oxime

A solution of Na₂CO₃ (460 mg, 4.34 mmol) in H₂O (4 mL) was added to a solution of 1-(2,5-dimethyloxazol-4-yl)-4-methylpentan-1-one (423 mg, 2.17 mmol) and NH₂OH HCl (300 mg, 4.34 mmol) in EtOH (10 mL). The mixture was then refluxed overnight, and concentrated under reduce pressure. The resulting residue was poured into H₂O (10 mL) and extracted with EtOAc (10 mL×3). The combined EtOAc extracts were dried over Na₂SO₄, and concentrated under reduced pressure to get 1-(2,5-dimethyloxazol-4-yl)-4-methylpentan-1-one oxime (370 mg, yield: 81.3%), which was used in the next step without further purification. LC-MS (022): 211.7 [M+H]⁺; Rt=1.466 min. Purity: 84.4% (254 nm)

### (d) 1-(2,5-dimethyloxazol-4-yl)-4-methylpentan-1-amine

Ra—Ni (21 mg) was added to a solution of 1-(2,5-dimethyloxazol-4-yl)-4-methylpentan-1-one oxime (210 mg, 1 mmol) in EtOH (10 mL). The mixture was stirred overnight under hydrogen atmosphere (1 atm). The precipitate was filtered off and filtrate was concentrated under reduced pressure to provide 1-(2,5-dimethyloxazol-4-yl)-4-methylpentan-1-amine (150 mg, yield: 76.5%), which was used in the next step without further purification. LC-MS (026): 180.7 [M−NH₂]⁺; Rt=1.548 min. Purity: 90% (214 nm).

### (e) 2-(2-((3,5-dimethylisoxazol-4-yl)(hydroxy)methyl)benzofuran-5-yl)-N-(1-(2,5-dimethyloxazol-4-yl)-4-methylpentyl)acetamide

This compound was synthesized from 1-(2,5-dimethyloxazol-4-yl)-4-methylpentan-1-amine and 2-(2-((3,5-dimethylisoxazol-4-yl)(hydroxy)methyl)benzofuran-5-yl)acetic acid essentially as described in example 1 (k) (35 mg, yield: 73%). LC-MS (022): 480.2 [M+H]⁺; Rt=1.468 min. Purity: 93% (254 nm). ¹H NMR (CDCl₃, 400 MHz): δ 7.40 (d, J=1.2 Hz, 1H), 7.37 (d, J=8.4 Hz, 1H), 7.14 (dd, J=1.2 Hz, 8.0 Hz, 1H), 6.51 (s, 1H), 5.99 (d, J=8.0 Hz, 1H), 5.85 (d, J=4.4 Hz, 1H), 4.78 (q, J=8.4 Hz, 1H), 3.61 (d, J=15.6 Hz, 1H), 3.56 (d, J=15.6 Hz, 1H), 2.38 (s, 3H), 2.33 (d, J=2.4 Hz, 3H), 2.25 (s, 6H), 1.70-1.60 (m, 2H), 1.50-1.42 (m, 1H), 1.06-0.96 (m, 2H), 0.79 (d, J=6.8 Hz, 6H).

### Example 49

### 2-(4-((2-(2-((3,5-dimethylisoxazol-4-yl)(hydroxy)methyl)benzofuran-5-yl)acetamido)(phenyl)methyl)-3-methylphenoxy)acetic acid

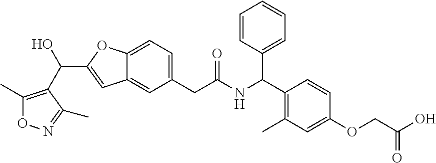 [SMILES: Cc1cc(OCC(=O)O)ccc1C(NC(=O)Cc1ccc2oc(C(O)c3c(C)noc3C)cc2c1)c1ccccc1]

### (a) ethyl 2-(4-((2-(2-((3,5-dimethylisoxazol-4-yl)(hydroxy)methyl)benzofuran-5-yl)acetamido)(phenyl)methyl)-3-methylphenoxy)acetate

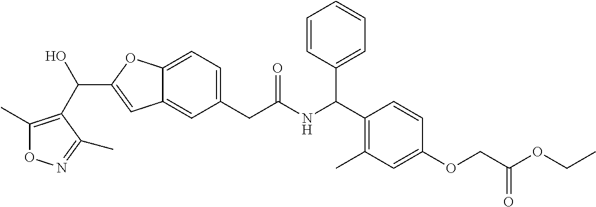 [SMILES: CCOC(=O)COc1ccc(C(NC(=O)Cc2ccc3oc(C(O)c4c(C)noc4C)cc3c2)c2ccccc2)c(C)c1]

This compound was synthesized from 2-(2-((3,5-dimethylisoxazol-4-yl)(hydroxy)methyl)benzofuran-5-yl)-N-((4-hydroxy-2-methylphenyl)(phenyl)methyl)acetamide essentially as described in example 36 (b). LC-MSA044: 583.7 [M+H]⁺; Rt=2.12 min, purity 82.04% (214 nm).

### (b) 2-(4-((2-(2-((3,5-dimethylisoxazol-4-yl)(hydroxy)methyl)benzofuran-5-yl)acetamido)(phenyl)methyl)-3-methylphenoxy)acetic acid

This compound was synthesized from ethyl 2-(4-((2-(2-((3,5-dimethylisoxazol-4-yl)(hydroxy)methyl)benzofuran-5-yl)acetamido)(phenyl)methyl)-3-methylphenoxy)acetate essentially as described in example 1 (j) (5 mg, yield: 17.8%). LC-MSA039: 555.7[M+H]⁺; Rt=1.324 min, purity 100% (214 nm). 1H NMR (CDCL3, 400 MHz): δ 7.42 (s, 1H), 7.39 (d, J=4.0 Hz, 2H), 7.26-7.22 (m, 1H), 7.17 (d, J=8.8 Hz, 1H), 7.05 (d, J=7.2 Hz, 2H), 6.74-6.69 (t, J=8.8 Hz, 2H), 6.57-6.51 (m, 2H), 6.31 (d, J=7.2 Hz, 1H), 5.89-5.85 (m, 2H), 5.34 (s, 1H), 4.60 (br, 2H), 3.70 (br, 2H), 2.40 (s, 3H), 2.24 (s, 3H), 2.18 (br, 3H).

### Example 50

### 2-(2-((3,5-dimethylisoxazol-4-yl)(hydroxy)methyl)benzofuran-5-yl)-N-((2-hydroxy-4,6-dimethoxyphenyl)(phenyl)methyl)acetamide

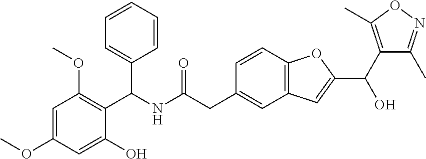

### (a) 2-(amino(phenyl)methyl)-3,5-dimethoxyphenol

This compound was synthesized from phenylmagnesium bromide and 2-hydroxy-4,6-dimethoxybenzonitrile essentially as described in example 1 (e) (60 mg, yield: 68.9%). LC-MS (036): 243.7 [M−OH]; Rt: 1.15 min.

### (b) 2-((2-(2-((3,5-dimethylisoxazol-4-yl)(hydroxy)methyl)benzofuran-5-yl)acetamido)(phenyl)methyl)-3,5-dimethoxyphenyl 2-(2-((3,5-dimethylisoxazol-4-yl)(hydroxy)methyl)benzofuran-5-yl)acetate

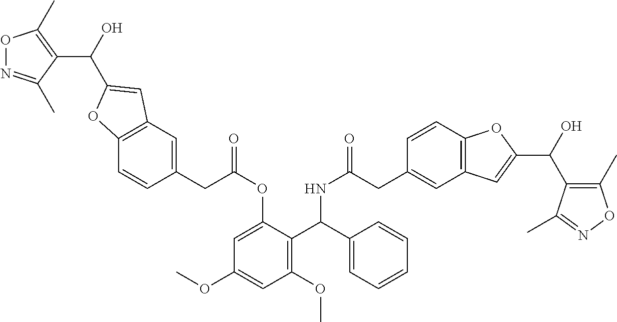 [SMILES: COc1cc(OC)c(C(NC(=O)Cc2ccc3oc(C(O)c4c(C)noc4C)cc3c2)c2ccccc2)c(OC(=O)Cc2ccc3oc(C(O)c4c(C)noc4C)cc3c2)c1]

This compound was synthesized from 2-(amino(phenyl)methyl)-3,5-dimethoxyphenol and 2-(2-((3,5-dimethylisoxazol-4-yl)(hydroxy)methyl)benzofuran-5-yl)acetic acid essentially as described in example 1 (k) (15 mg 7.8%) as solid. LC-MS (036): 809.2 [M+H]; Rt: 1.61 min.

### (c) 2-(2-((3,5-dimethylisoxazol-4-yl)(hydroxy)methyl)benzofuran-5-yl)-N-((2-hydroxy-4,6-dimethoxyphenyl)(phenyl)methyl)acetamide

This compound was synthesized from 2-((2-((2-((3,5-dimethylisoxazol-4-yl)(hydroxy)methyl)benzofuran-5-yl)acetamido)(phenyl)methyl)-3,5-dimethoxyphenyl 2-(2-((3,5-dimethylisoxazol-4-yl)(hydroxy)methyl)benzofuran-5-yl)acetate essentially as described in example 1 (j) (3 mg, yield 46.1%). LC-MSA039: 542.0 [M+H]⁺; Rt=1.65 min, purity 98% (254 nm). ¹H NMR (MeOD, 400 MHz): δ 7.55 (s, 1H), 7.48-7.46 (d, J=8.4 Hz, 1H), 7.26-7.24 (d, J=8.8 Hz, 1H), 7.16-7.10 (m, 5H), 6.76-6.73 (d, J=8.8 Hz, 2H), 6.0 (s, 2H), 5.90 (s, 1H), 3.74 (s, 2H), 3.72 (s, 3H), 3.46 (s, 3H), 2.40 (s, 3H), 2.23 (s, 3H).

### Example 51

### 2-(2-((3,5-dimethylisoxazol-4-yl)(hydroxy)methyl)-6-fluorofuro[3,2-b]pyridin-5-yl)-N-((2,4-dimethylphenyl)(phenyl)methyl)acetamide

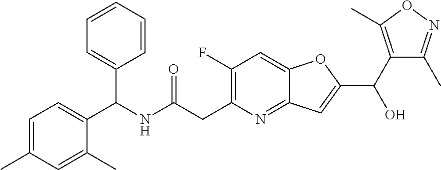

### (a) 5-(benzyloxy)-2-chloro-3-fluoropyridine

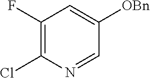 [SMILES: Fc1cc(OCc2ccccc2)cnc1Cl]

NaH (60% in mineral, 1.47 g, 36.7 mmol) was added portion-wise to a solution of 6-chloro-5-fluoropyridin-3-ol (4.5 g, 30.6 mmol) in DMF (40 ml) at 0° C. The reaction mixture was stirred at rt for 1 h, then benzyl bromide (6.24 g, 36.7 mmol) was added drop-wise and the mixture was stirred at rt for another 4 h. Water (100 ml) was added then slowly, and the mixture was extracted with EtOAc (300 ml). The extracts were washed with brine (400 ml), dried over Na₂SO₄, and concentrated under reduced pressure to yield Removal of solvents afforded 5-(benzyloxy)-2-chloro-3-fluoropyridine (6.9 g, yield: 95%) as a yellow solid. LCMSA036: 238[MH]⁺; Rt: 1.84 min

### (b) 5-(benzyloxy)-3-fluoro-2-methylpyridine

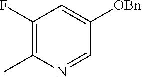 [SMILES: Cc1ncc(OCc2ccccc2)cc1F]

Pd(dppf)Cl₂ (1.9 g, 2.32 mmol) was added to a solution of 5-(benzyloxy)-2-chloro-3-fluoropyridine (5.5 g, 23.2 mmol), 2,4,6-trimethyl-1,3,5,2,4,6-trioxatriborinane (7.1 g, 46.4 mmol), and K₂CO₃ (6.4 g, 46.4 mmol) in dioxane (100 ml) and water (10 ml) at rt. The reaction mixture stirred at 100° C. under N₂ atmosphere overnight. Water (200 ml) was then added, and the mixture was extracted with EtOAc (300 ml). The EtOAc phase was washed with brine (400 ml), dried over Na₂SO₄, and concentrated under reduced pressure. The resulting residue was purified by silica gel column (Petroleum ether:EtOAc=20:1) to afford 5-(benzyloxy)-3-fluoro-2-methylpyridine (2.4 g, yield: 48%) as a white solid. LCMSA036: 218[MH]⁺; Rt: 1.70 min

### (c) methyl 2-(5-(benzyloxy)-3-fluoropyridin-2-yl)acetate

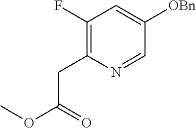 [SMILES: COC(=O)Cc1ncc(OCc2ccccc2)cc1F]

LDA (2M, 9.1 ml, 18.3 mmol) was added to a solution of 5-(benzyloxy)-3-fluoro-2-methylpyridine (2.65 g, 12.2 mmol) and TMEDA (2.1 g, 18.3 mmol) in THF (50 ml) at −78° C. under N₂ atmosphere. The reaction mixture was stirred at −78° C. for 1 h, then dimethyl carbonate (4.4 g, 48.8 mmol) was added drop-wise and the mixture was stirred at −78° C. for an additional 2 h. Water (100 ml) was then added, and the mixture was extracted with EtOAc (200 ml). The organic phase was separated and washed with brine (400 ml), dried over Na₂SO₄, and concentrated under reduced pressure. The resulting residue was purified by silica gel column (Petroleum ether:EtOAc=10:1) to afford methyl 2-(5-(benzyloxy)-3-fluoropyridin-2-yl)acetate (2 g, yield: 59%) as a white solid. LCMSA036: 276[MH]⁺; Rt: 1.71 min

### (d) methyl 2-(3-fluoro-5-hydroxypyridin-2-yl)acetate

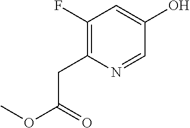

A mixture of methyl 2-(5-(benzyloxy)-3-fluoropyridin-2-yl)acetate (2 g, 7.3 mmol) and 10% Pd/C (200 mg) in MeOH (50 mL) was stirred at rt under H₂ atmosphere for 4 h. The reaction was then filtered through a pad of Celite. The filtrate was concentrated under reduced pressure to afford desired product methyl 2-(3-fluoro-5-hydroxypyridin-2-yl)acetate (1.3 g, yield: 97%) as a yellow solid. LCMSA020: 186 [M+H]⁺; Rt: 1.13 min

### (e) methyl 2-(3-fluoro-5-hydroxy-6-iodopyridin-2-yl)acetate

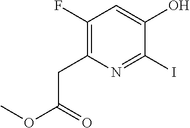

A mixture of methyl 2-(3-fluoro-5-hydroxypyridin-2-yl)acetate (1.2 g, 6.5 mmol) and Na₂CO₃ (1.03 g, 9.8 mmol) in water (50 ml) was stirred at rt for 30 min. I₂ (1.65 g, 6.5 mmol) was added, and the mixture was stirred at rt for 2 h. The reaction mixture was then acidified to pH˜6-7 with diluted hydrochloric acid. The mixture was extracted with EtOAc (300 ml). The combined extracts were washed with brine (400 ml), dried over Na₂SO₄, and concentrated under reduced pressure to yield methyl 2-(3-fluoro-5-hydroxy-6-iodopyridin-2-yl)acetate (1.7 g, 84%) as a red solid, which was used in the next step without further purification. LCMSA027: 312[M+H]⁺; Rt: 1.14 min

### (f) methyl 2-(2-((3,5-dimethylisoxazol-4-yl)(hydroxy)methyl)-6-fluorofuro[3,2-b]pyridin-5-yl)acetate

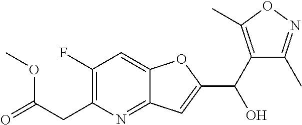 [SMILES: COC(=O)Cc1nc2cc(C(O)c3c(C)noc3C)oc2cc1F]

A mixture of methyl 2-(3-fluoro-5-hydroxy-6-iodopyridin-2-yl)acetate (100 mg, 0.32 mmol), 1-(3,5-dimethylisoxazol-4-yl)prop-2-yn-1-ol (73 mg, 0.48 mmol), Pd(PPh₃)₂Cl₂ (22 mg, 0.03 mmol) and CuI (6 mg, 0.03 mmol) in Et₃N (7 mL) was stirred under N₂ atmosphere at 90° C. overnight. Water (10 ml) was then added, and the mixture was extracted with EtOAc (30 ml). The combined extracts were washed with brine (40 ml), dried over Na2SO4, and concentrated under reduced pressure. The resulting residue was purified by P-TLC (Petroleum ether:EtOAc=5:1) to afford methyl 2-(2-((3,5-dimethylisoxazol-4-yl)(hydroxy)methyl)-6-fluorofuro[3,2-b]pyridin-5-yl)acetate (70 mg, yield: 65%) as a yellow oil. LCMSA020: 335[M+H]⁺; Rt: 1.45 min

### (g) 2-(2-((3,5-dimethylisoxazol-4-yl)(hydroxy)methyl)-6-fluorofuro[3,2-b]pyridin-5-yl)acetic acid

This compound was synthesized from methyl 2-(2-((3,5-dimethylisoxazol-4-yl)(hydroxy)methyl)-6-fluorofuro[3,2-b]pyridin-5-yl)acetate essentially as described in example 1 (j) (90 mg, crude). LCMSA039: 321[M+H]⁺; Rt: 1.05 min

### (h) 2-(2-((3,5-dimethylisoxazol-4-yl)(hydroxy)methyl)-6-fluorofuro[3,2-b]pyridin-5-yl)-N-((2,4-dimethylphenyl)(phenyl)methyl)acetamide

This compound was synthesized from 2-(2-((3,5-dimethylisoxazol-4-yl)(hydroxy)methyl)-6-fluorofuro[3,2-b]pyridin-5-yl)acetic acid and (2,4-dimethylphenyl)(phenyl)methanamine essentially as described in example 1 (j) (10 mg, yield: 7%). LCMSA020: 514[M+H]⁺; Rt: 1.72 min. ¹H NMR (MeOD, 400 MHz): δ 9.65 (d, J=8.0 Hz 1H), 7.38-7.25 (m, 7H), 7.07 (s, 1H), 7.01-6.95 (m, 3H), 6.58-6.56 (m, 1H), 5.92 (s, 1H), 2.61 (d, J=3.2 Hz, 3H), 2.34-2.31 (m, 6H), 2.20 (s, 3H)

### Example 52

### 2-(2-((3,5-dimethylisoxazol-4-yl)(hydroxy)methyl)benzofuran-5-yl)-1-(1-(4-fluorophenyl)-6-methyl-3,4-dihydroisoquinolin-2(1H)-yl)ethanone

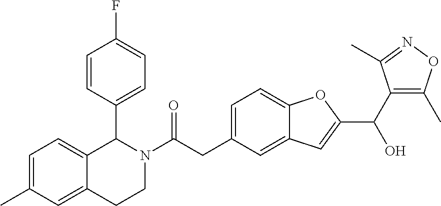

### (a) 1-(4-fluorophenyl)-6-methyl-1,2,3,4-tetrahydroisoquinoline

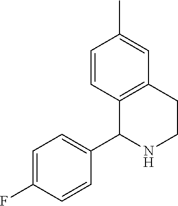 [SMILES: Cc1ccc2c(c1)CCNC2c1ccc(F)cc1]

A solution of 2-(m-tolyl)ethanamine (1.35 g, 10 mmol) and 4-fluorobenzaldehyde (1.24 g, 10 mmol) in EtOH (20 mL) was refluxed for 2 h. The reaction mixture was concentrated under reduced pressure, and trifluoromethanesulfonic acid (20 mL) was added to the residue. The resulting solution was stirred at 60° C. overnight. After cooling to rt, the mixture was poured into ice water and basified with 1N NaOH to pH˜11. The mixture was extracted with CH₂Cl₂ (30 mL×3). The combined extracts were washed with brine (10 mL), dried over Na₂SO₄, and concentrated under reduced pressure. The resulting residue was purified by flash column (petroleum ether/EtOAc 5/1) to give 1-(4-fluorophenyl)-6-methyl-1,2,3,4-tetrahydroisoquinoline (935 mg, yield: 32%).

### (b) 2-(2-((3,5-dimethylisoxazol-4-yl)(hydroxy)methyl)benzofuran-5-yl)-1-(1-(4-fluorophenyl)-6-methyl-3,4-dihydroisoquinolin-2(1H)-yl)ethanone

This compound was synthesized from 1-(4-fluorophenyl)-6-methyl-1,2,3,4-tetrahydroisoquinoline and 2-(2-((3,5-dimethylisoxazol-4-yl)(hydroxy)methyl)benzofuran-5-yl)acetic acid essentially as described in example 1 (k) (53 mg, yield: 38.9%). LC-MS (020): 525.2 [M+H]⁺; Rt: 1.95 min. ¹H-NMR (MeOD, 400 MHz): δ 7.42-7.37 (m, 2H), 7.20-7.15 (m, 3H), 7.01-6.89 (m, 5H), 6.81 (s, 1H), 6.67 (s, 1H), 5.87 (s, 1H), 3.96-3.94 (m, 3H), 3.33-3.32 (m, 1H), 2.66-2.64 (m, 2H), 2.38 (s, 3H), 2.31 (s, 3H), 2.21 (s, 3H).

### Example 53

### N—((S)-(2,4-dimethylphenyl)(phenyl)methyl)-2-(2-(hydroxy(pyridin-4-yl)methyl)furo[3,2-b]pyridin-5-yl)acetamide

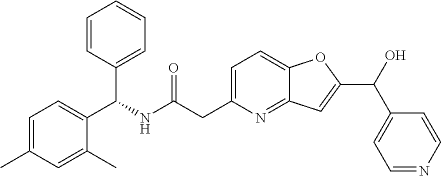 [SMILES: Cc1ccc([C@@H](NC(=O)Cc2ccc3oc(C(O)c4ccncc4)cc3n2)c2ccccc2)c(C)c1]

### (a) Ethyl 2-(5-hydroxy-6-iodopyridin-2-yl)acetate

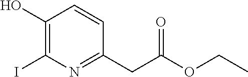

Iodine (716.3 mg, 2.82 mmol) was added to a solution of ethyl 2-(5-hydroxypyridin-2-yl)acetate (510 mg, 2.82 mmol) and sodium carbonate (627.5 mg, 5.92 mmol) in water (20 mL) under nitrogen atmosphere, and the mixture was stirred at rt for 2 h. The reaction mixture was extracted with EtOAc, washed with brine, and dried over sodium sulfate. Removal of solvent gave 600 mg of crude ethyl 2-(5-hydroxy-6-iodopyridin-2-yl)acetate, which was carried through without further purification. LCMS-027: 308.0 [M+H]⁺; Rt: 1.11 min

### (b) 1-(pyridin-4-yl)prop-2-yn-1-ol

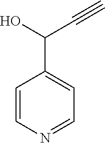

TBAF (1M solution, 4.87 mL, 4.87 mmol) was added to a solution of 1-(pyridin-4-yl)-3-(trimethylsilyl)prop-2-yn-1-ol (500 mg, 2.43 mmol) in THF (10 ml) the under N₂ atmosphere. Then the mixture was stirred at rt for 2 h, extracted with EtOAc (3×40 mL). The combined extracts were concentrated under reduced pressure to yield 1-(pyridin-4-yl)prop-2-yn-1-ol (392 mg, crude), which was used in the next step without further purification. LC-MS (027): 134.1 [M+H]⁺; Rt: 0.24 min.

### (c) ethyl 2-(2-isonicotinoylfuro[3,2-b]pyridin-5-yl)acetate

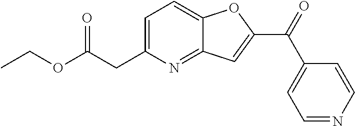 [SMILES: CCOC(=O)Cc1ccc2oc(C(=O)c3ccncc3)cc2n1]

This compound was synthesized from ethyl 2-(5-hydroxy-6-iodopyridin-2-yl)acetate and 1-(pyridin-4-yl)prop-2-yn-1-ol essentially as described in example 51 (f) (21 mg, 9.37%). LC-MS (036): 311.1 [M+H]⁺; Rt: 1.43 min; Purity: 67.2% (254 nm).

### (d) 2-(2-isonicotinoylfuro[3,2-b]pyridin-5-yl)acetic acid

This compound was synthesized from ethyl 2-(2-isonicotinoylfuro[3,2-b]pyridin-5-yl)acetate essentially as described in example 1(j). LC-MS (039): 283.2 [M+H]⁺; Rt: 1.08 min; Purity: 69.4%

### (e) (S)—N-((2,4-dimethylphenyl)(phenyl)methyl)-2-(2-isonicotinoylfuro[3,2-b]pyridin-5-yl)acetamide

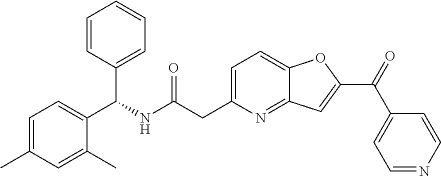 [SMILES: Cc1ccc([C@@H](NC(=O)Cc2ccc3oc(C(=O)c4ccncc4)cc3n2)c2ccccc2)c(C)c1]

This compound was synthesized from (S)-(2,4-dimethylphenyl)(phenyl)methanamine and 2-(2-isonicotinoylfuro[3,2-b]pyridin-5-yl)acetic acid essentially as described in example 1(k) (9 mg, 12%). LC-MS (039): 476.2 [M+H]⁺; Rt: 1.70 min; Purity: 51.0%

### (f) N—((S)-(2,4-dimethylphenyl)(phenyl)methyl)-2-(2-(hydroxy(pyridin-4-yl)methyl)furo[3,2-b]pyridin-5-yl)acetamide

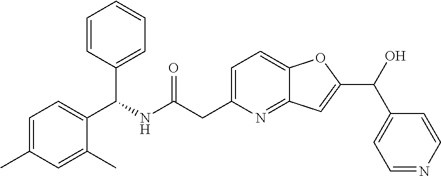 [SMILES: Cc1ccc([C@@H](NC(=O)Cc2ccc3oc(C(O)c4ccncc4)cc3n2)c2ccccc2)c(C)c1]

This compound was synthesized from (S)—N-((2,4-dimethylphenyl)(phenyl)methyl)-2-(2-isonicotinoylfuro[3,2-b]pyridin-5-yl)acetamide essentially as described in example 38 (f) (3 mg, 34%). LC-MS (039): 478.3 [M+H]⁺; Rt: 1.16 min; Purity: 100%. ¹H-NMR (MeOD, 400 MHz): δ 8.57 (d, J=6 Hz, 2H), 7.83 (d, J=8.8 Hz, 1H), 7.61 (d, J=4.8 Hz, 2H), 7.31-7.19 (m, 6H), 7.04-6.97 (m, 3H), 6.87 (s, 1H), 6.32 (s, 1H), 6.03 (s, 1H), 3.90 (s, 2H), 2.29 (s, 3H), 2.21 (s, 3H)

### Example 54

### 2-(2-((S)-(3,5-dimethylisoxazol-4-yl)(hydroxy)methyl)benzofuran-5-yl)-N-((4-isocyano-2-methylphenyl)(phenyl)methyl)acetamide

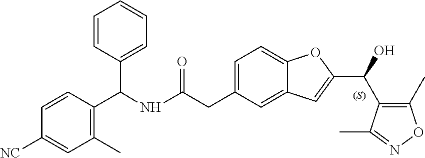

### (a) (4-bromo-2-methylphenyl)(phenyl)methanamine

To a stirred solution of 4-bromo-2-methylbenzonitrile (400 mg, 2.0 mmol) in THF (5 mL) was added dropwise a solution of phenylmagnesium bromide (1M in THF, 2.5 mL) at 0° C. under nitrogen. The cooling bath was removed and the reaction mixture was allowed to warm to rt. After 1 h stirring at rt, the reaction mixture was heated to 40° C. for 16 h. Once the reaction mixture was allowed to cool to rt, the solvent was removed under reduced pressure. To the resulting oil was added methanol (5 mL) and the reaction mixture was cooled to 0° C. Sodium borohydride (81 mg, 2.1 mmol) was added portionwise to the reaction mixture. After the addition was complete, the cooling bath was removed and the reaction mixture was allowed to warm to rt and stirred for 18 h. The solvent was removed under reduced pressure and the remaining residue was slurried in ethyl acetate (50 mL) and water (30 mL). The layers were separated and the ethyl acetate layer was washed with water (20 mL), brine (20 mL), dried over Na₂SO₄, and concentrated. The resulting oil was purified by on a silica gel column (1:1 hexanes/EtOAc) to obtain the title compound (180 mg, 32%) as a yellow oil. LCMS-TFA: 276 [M+H]⁺; Rₜ: 3.937 min. ¹H NMR (400 MHz, CDCl₃) δ ppm 7.45 (d, J=8.0 Hz, 1H), 7.36 (d, J=8.0 Hz, 2H), 7.27 (m, 6H), 5.31 (s, 1H), 2.21 (s, 3H).

### (b) N-((4-bromo-2-methylphenyl)(phenyl)methyl)-2-(2-((S)-(3,5-dimethylisoxazol-4-yl)(hydroxy)methyl)benzofuran-5-yl)acetamide

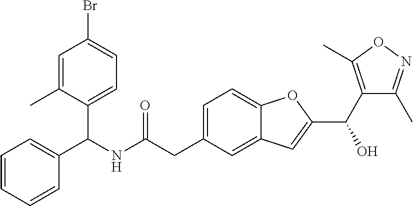 [SMILES: Cc1cc(Br)ccc1C(NC(=O)Cc1ccc2oc([C@@H](O)c3c(C)noc3C)cc2c1)c1ccccc1]

To a stirred suspension of (S)-2-(2-((3,5-dimethylisoxazol-4-yl)(hydroxyl)methyl)benzofuran-5-yl)acetic acid (34 mg, 0.11 mmol) and HATU (52 mg, 0.13 mmol) in CH₂Cl₂ was added DIPEA (30 mg, 0.2 mmol). The resulting solution was allowed to stir at rt for 20 min and then (4-bromo-2-methylphenyl)(phenyl)methanamine (43 mg, 0.16 mmol) was added to the solution. The resulting mixture was stirred at rt for 14 h. CH₂Cl₂ (20 mL) was added to the mixture and the mixture was washed with 50% aqueous sodium bicarbonate solution (10 mL). After separating the layers, the aqueous layer was back extracted with CH₂Cl₂ (20 mL). The CH₂Cl₂ extracts were combined and solution was concentrated under reduced pressure. The title compound was isolated as a white solid (43 mg, 68%). LCMS-AMF: 561 [M+H]⁺; Rₜ: 6.048 min.

### c) N-((4-cyano-2-methylphenyl)(phenyl)methyl)-2-(2-((S)-(3,5-dimethylisoxazol-4-yl)(hydroxy)methyl)benzofuran-5-yl)acetamide

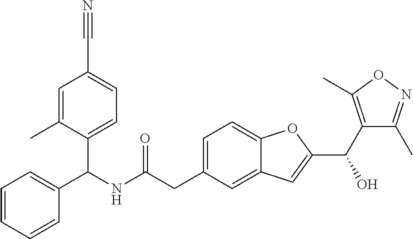 [SMILES: Cc1cc(C#N)ccc1C(NC(=O)Cc1ccc2oc([C@@H](O)c3c(C)noc3C)cc2c1)c1ccccc1]

To a stirred solution of N-((4-bromo-2-methylphenyl)(phenyl)methyl)-2-(2-((S)-(3,5-dimethylisoxazol-4-yl)(hydroxy)methyl)benzofuran-5-yl)acetamide (43 mg, 0.08 mmol) in DMF (1 mL) was added cupric (I) cyanide (8 mg, 0.09 mmol) and the suspension was sealed and heated to 200° C. for 2 h. The reaction mixture was filtered through a pad of Celite and washed with CH₂Cl₂ (15 mL), EtOAc (10 mL), MeOH (10 mL) and MeCN (10 mL). The washes were combined and concentrated under reduced pressure. The concentrated residue was purified by preparative HPLC to obtain the title compound (6 mg, 15%) as a yellow solid. LCMS-AMF: 506 [M+H]⁺; Rₜ: 5.436 min. ¹H NMR (400 MHz, CDCl₃) δ ppm 7.43 (m, 4H), 7.18 (d, J=8.0 Hz, 1H), 7.10 (d, J=8.0 Hz, 1H), 7.00 (t, J=4.0 Hz, 2H), 6.56 (s, 1H), 6.33 (d, J=8.0 Hz, 1H), 5.93 (d, J=8.0 Hz, 1H), 5.89 (s, 1H), 3.72 (s, 2H), 2.49 (bs, 1H), 2.40 (s, 3H), 2.27 (s, 3H), 2.23 (s, 3H).

### Example 55

### 2-(2-((S)-(3,5-dimethylisoxazol-4-yl)(hydroxy)methyl)benzofuran-5-yl)-N-((3-methylpyridin-4-yl)(phenyl)methyl)acetamide

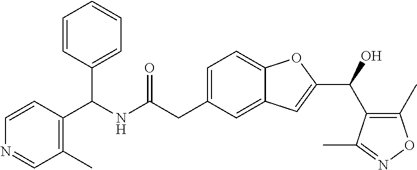 [SMILES: Cc1cnccc1C(NC(=O)Cc1ccc2oc([C@@H](O)c3c(C)noc3C)cc2c1)c1ccccc1]

### a) (3-methylpyridin-4-yl)(phenyl)methanamine

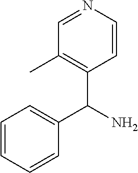

To a stirred solution of 3-methylisonicotinonitrile (240 mg, 2.0 mmol) in THF (3 mL) was added dropwise a solution of phenylmagnesium bromide (1M in THF, 2.5 mL) at 0° C. under nitrogen. The cooling bath was removed and the reaction mixture was allowed to warm to rt. After 1.5 h stirring at rt, the reaction mixture was heated to 40° C. for 14 h. Once the reaction mixture was allowed to cool to rt, the solvent was removed under reduced pressure. To the resulting oil was added methanol (3 mL) and the reaction mixture was cooled to 0° C. Sodium borohydride (80 mg, 2.0 mmol) was added portionwise to the reaction mixture. After the addition was complete, the cooling bath was removed and the reaction mixture was allowed to warm to rt and stirred for 12 h. The solvent was removed under reduced pressure and the remaining residue was slurried in ethyl acetate (40 mL) and water (25 mL). The layers were separated and the ethyl acetate layer was washed with water (20 mL), dried over Na₂SO₄, and concentrated. The resulting oil was purified by on a silica gel column (1:1 hexanes/EtOAc) to obtain the title compound (150 mg, 37%) as a waxy solid. LCMS-TFA: 199[M+H]⁺; Rₜ: 1.880 min. ¹H NMR (400 MHz, CDCl₃) δ ppm 8.49 (d, J=8.0 Hz, 1H), 8.32 (s, 1H), 7.57 (d, J=4.0 Hz, 1H), 7.29 (m, 5H), 5.30 (s, 1H), 2.17 (s, 3H).

### b) 2-(2-((S)-(3,5-dimethylisoxazol-4-yl)(hydroxy)methyl)benzofuran-5-yl)-N-((3-methylpyridin-4-yl)(phenyl)methyl)acetamide

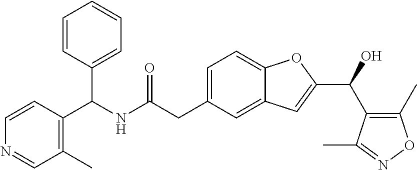 [SMILES: Cc1cnccc1C(NC(=O)Cc1ccc2oc([C@@H](O)c3c(C)noc3C)cc2c1)c1ccccc1]

To a stirred suspension of (S)-2-(2-((3,5-dimethylisoxazol-4-yl)(hydroxyl)methyl)benzofuran-5-yl)acetic acid (40 mg, 0.13 mmol) and HATU (66 mg, 0.17 mmol) in CH₂Cl₂ (1 mL) was added DIPEA (37 mg, 0.27 mmol). The resulting mixture was allowed to stir at rt for 15 min and then (3-methylpyridin-4-yl)(phenyl)methanamine (40 mg, 0.2 mmol) was added to the solution. The resulting mixture was stirred at rt for 12 h. CH₂Cl₂ (20 mL) was added to the mixture and the mixture was washed with 50% aqueous sodium bicarbonate solution (15 mL). After separating the layers, the aqueous layer was back extracted with CH₂Cl₂ (15 mL). The CH₂Cl₂ extracts were combined, dried over Na₂SO₄ and the solution was concentrated under reduced pressure. The concentrated residue was purified by preparative HPLC to obtain the title compound (6 mg, 10%) as a white solid. LCMS-TFA: 482 [M+H]⁺; Rₜ: 3.943 min. ¹H NMR (400 MHz, CDCl₃) δ ppm 8.33 (d, J=4.0 Hz, 2H), 7.43 (d, J=8.0 Hz, 2H), 7.17 (m, 1H), 7.01 (m, 2H), 6.88 (s, 1H), 6.57 (d, J=4.0 Hz, 1H), 6.28 (d, J=8.0 Hz, 1H), 5.98 (d, J=8.0 Hz, 1H), 5.88 (s, 1H), 3.72 (s, 2H), 2.39 (s, 3H), 2.25 (s, 3H), 2.14 (s, 3H).

### Example 56

### 2-(2-((3,5-dimethylisoxazol-4-yl)(hydroxy)methyl)benzofuran-5-yl)-1-(5-phenyl-2,3-dihydrobenzo[f][1,4]oxazepin-4(5H)-yl)ethanone

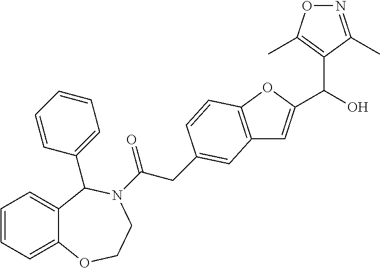 [SMILES: Cc1noc(C)c1C(O)c1cc2cc(CC(=O)N3CCOc4ccccc4C3c3ccccc3)ccc2o1]

To a solution of 5-phenyl-2,3,4,5-tetrahydrobenzo[f][1,4]oxazepine (36 mg, 0.16 mmol), THF (1 mL), DMAP (39 mg, 0.32 mmol) and 2-(2-((3,5-dimethylisoxazol-4-yl)(hydroxy)methyl)benzofuran-5-yl)acetic acid (57.8 mg, 0.19 mmol), 184 mg T3P solution (50% w/w) was added were added dropwise and stirred at rt for 2 h. After the reaction was complete it was quenched with 1N sodium hydroxide. The organics were diluted with ethyl acetate and washed with water brine solution. After drying over sodium sulfate, the solvent was concentrated under reduced pressure and the oil was purified by flash chromatography. (Hexanes/EtOAc gradient) to obtain the title compound (65 mg, yield 80%) as a white solid. LCMS: 509 [M+H]⁺; Rₜ=5.77 min. ¹H NMR (400 MHz, CDCl₃) δ ppm 7.34-7.31 (m, 2H), 7.19-7.09 (m, 4H), 6.97 (d, J=7.2 Hz, 2H), 6.89 (s, 1H), 6.81 (d, J=8.0 Hz, 1H), 6.67 (d, J=8.0 Hz, 1H), 6.46 (d, J=8.0 Hz, 1H), 6.46 (s, 1H), 6.28 (d, J=8.0 Hz, 1H), 5.90 (d, J=8.0 Hz, 1H), 5.77 (s, 1H), 3.61 (s, 2H), 2.82 (br, 1H), 2.30 (s, 3H), 2.20 (s, 3H), 2.37-2.38 (m, 3H), 2.25-2.25 (m, 3H).

Following essentially the procedure as described in Example 55, the compound in Table 1 was prepared.

TABLE 1

| Example | Structure/Name | NMR | LCMS |
|---|---|---|---|
| 57 | 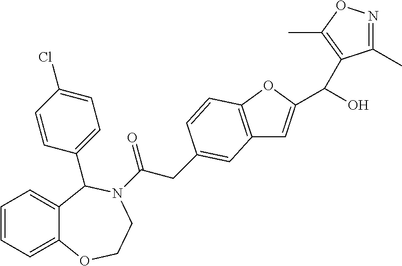 [SMILES: Cc1noc(C)c1C(O)c1cc2cc(CC(=O)N3CCOc4ccccc4C3c3ccc(Cl)cc3)ccc2o1] | ¹H NMR (400 MHz, CDCl₃-d) d ppm 7.36-7.44 (m, 2 H) 7.27- 7.25 (m, 3 H) 7.05-7.16 (m, 2 H) 6.99-7.04 (m, 1 H) 6.93 (d, J = 8.06 Hz, 1 H) 6.69 (dd, J = 8.06, 4.15 Hz, 1 H) 6.48-6.56 (m, 1 H) 6.15 (s, 1 H) 5.87 (t, J = 3.91 Hz, 1 H) 4.55-4.6 (m, 1 H) 4.13-4.21 (m, 1 H) 3.91-4.10 (m, 1 H) 3.78- 3.91 (m, 1 H) 3.45-3.57 (m, 1 H) 3.35-3.45 (m, 1 H) 2.99- 3.09 (m, 1 H) 2.46 (d, J = 3.42 Hz, 1 H) 2.34-2.42 (m, 3 H) 2.21- 2.27 (m, 3 H) | LCMS-P1: 543 [M + H]⁺; Rₜ = 6.177 min |
| | 1-(5-(4-chlorophenyl)-2,3- | | |
| | dihydrobenzo[f][1,4]oxazepin- | | |
| | 4(5H)-yl)-2-(2-((3,5- | | |
| | dimethylisoxazol-4- | | |
| | yl)(hydroxy)methyl)benzofuran- | | |
| | 5-yl)ethanone | | |

### Example 58

### 2-(2-((3,5-dimethylisoxazol-4-yl)(hydroxy)methyl)benzofuran-5-yl)-N-((4-(hydroxymethyl)-2-methylphenyl)(phenyl)methyl)acetamide

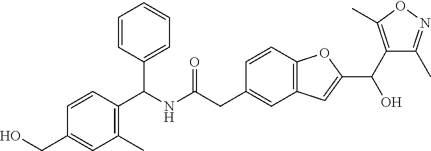 [SMILES: Cc1cc(CO)ccc1C(NC(=O)Cc1ccc2oc(C(O)c3c(C)noc3C)cc2c1)c1ccccc1]

### a) methyl 4-cyano-3-methylbenzoate

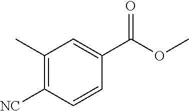

This compound was synthesized from methyl 4-bromo-3-methylbenzoate essentially as described in example 38 (a) (4 g, yield 46%). LC-MS (038): 176.1 [M+H]⁺; Rt=1.698 min.

### b) 4-(hydroxymethyl)-2-methylbenzonitrile

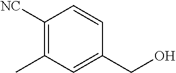

LiCl (722 mg, 17 mmol), and NaBH₄ (651 mg, 17 mmol) were added portion-wise to a solution of methyl 4-cyano-3-methylbenzoate (2 g, 11 mmol) in 50 mL of MeOH. After stirring at RT overnight, the mixture was quenched with NH₄Cl (aq.) (10 mL). The mixture was then extracted with EtOAc (20 mL*3). The combined extracts were washed with brine (10 mL), dried over Na₂SO₄, and concentrated under reduced pressure. The resulting residue was purified by flash column (petroleum ether/EtOAc=10/1 to 3/1) to give 4-(hydroxymethyl)-2-methylbenzonitrile (1.4 g, yield: 87%). LC-MS (038): 148.1 [M+H]⁺; Rt=1.454 min.

### c) (4-(amino(phenyl)methyl)-3-methylphenyl)methanol

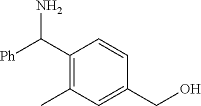 [SMILES: Cc1cc(CO)ccc1C(N)c1ccccc1]

This compound was synthesized from 4-(hydroxymethyl)-2-methylbenzonitrile and phenylmagnesium bromide essentially as described in example 1 (e) (347 mg, Yield: 90.1%). LC-MS (036): 211 [M−NH₂]⁺; Rt=1.13 min.

### d) 2-(2-((3,5-dimethylisoxazol-4-yl)(hydroxy)methyl)benzofuran-5-yl)-N-((4-(hydroxymethyl)-2-methylphenyl)(phenyl)methyl)acetamide

This compound was synthesized from 4(4-(amino(phenyl)methyl)-3-methylphenyl)methanol and 2-(2-((3,5-dimethylisoxazol-4-yl)(hydroxy)methyl)benzofuran-5-yl)acetic acid essentially as described in example 1 (k) (85 mg, yield: 83%). LC-MS (044): 511 [M+H]⁺; Rt=1.67 min. ¹H NMR (DMSO, 400 MHz): δ 8.89 (d, J=8.4 Hz, 1H), 7.47-7.07 (m, 11H), 6.73 (s, 1H), 6.21-6.17 (m, 2H), 5.82 (d, J=4 Hz, 1H), 5.08 (t, J=5.6 Hz, 1H), 4.42 (d, J=6.4 Hz, 2H), 3.58 (s, 2H), 2.34 (s, 3H), 2.17 (s, 3H), 2.12 (s, 3H).

### Examples 59 and 60

### 1-(6-chloro-1-phenyl-3,4-dihydroisoquinolin-2(1H)-yl)-2-(2-((3,5-dimethylisoxazol-4-yl)(hydroxy)methyl)benzofuran-5-yl)ethanone and 1-(8-chloro-1-phenyl-3,4-dihydroisoquinolin-2(1H)-yl)-2-(2-((3,5-dimethylisoxazol-4-yl)(hydroxy)methyl)benzofuran-5-yl)ethanone

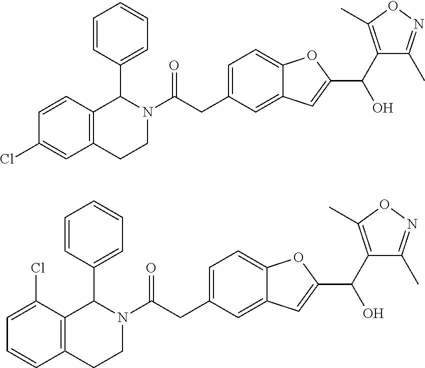 [SMILES: Cc1noc(C)c1C(O)c1cc2cc(CC(=O)N3CCc4cc(Cl)ccc4C3c3ccccc3)ccc2o1 ; Cc1noc(C)c1C(O)c1cc2cc(CC(=O)N3CCc4cccc(Cl)c4C3c3ccccc3)ccc2o1]

### a) N-benzylidene-2-(3-chlorophenyl)ethanamine

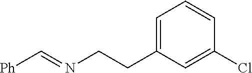 [SMILES: Clc1cccc(CC/N=C/c2ccccc2)c1]

A solution of 2-(3-chlorophenyl)ethanamine (1.55 g, 10 mmol) and benzaldehyde (1.06 g, 10 mmol) in 20 mL of EtOH was refluxed for 2 h. EtOH was then removed under reduced pressure and the residue was put under vacuum to remove all the volatile solvent and it was carried through without further purification (2.5 g, yield 100%).

### b) 6-chloro-1-phenyl-1,2,3,4-tetrahydroisoquino line and 8-chloro-1-phenyl-1,2,3,4-tetrahydroisoquinoline

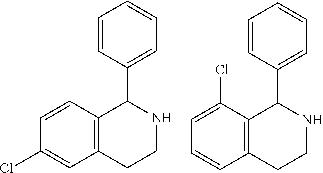

A solution of N-benzylidene-2-(3-chlorophenyl)ethanamine (2.4 g, 10 mmol) in 25 mL of trifluoromethanesulfonic acid was stirred at 90° C. overnight. After cooling to rt, the mixture was poured into ice-water and neutralized with 1 N NaOH to pH˜8. The mixture was extracted with CH₂Cl₂ (30 mL×3). The combined extracts were washed with brine (10 mL), dried over Na₂SO₄, and concentrated under reduced pressure. The resulting residue was purified by flash column (petroleum ether/EtOAc=4/1) to give 800 mg of 6-chloro-1-phenyl-1,2,3,4-tetrahydroisoquinoline and 8-chloro-1-phenyl-1,2,3,4-tetrahydroisoquinoline as a mixture (in a 2:1 ratio). LC-MS (038): 244.1 [M+H]⁺; Rt: 1.32 min for 8-chloro-1-phenyl-1,2,3,4-tetrahydroisoquinoline and 1.35 min for 6-chloro-1-phenyl-1,2,3,4-tetrahydroisoquinoline.

### c) 1-(6-chloro-1-phenyl-3,4-dihydroisoquinolin-2(1H)-yl)-2-(2-((3,5-dimethylisoxazol-4-yl)(hydroxy)methyl)benzofuran-5-yl)ethanone and 1-(8-chloro-1-phenyl-3,4-dihydroisoquinolin-2(1H)-yl)-2-(2-((3,5-dimethylisoxazol-4-yl)(hydroxy)methyl)benzofuran-5-yl)ethanone

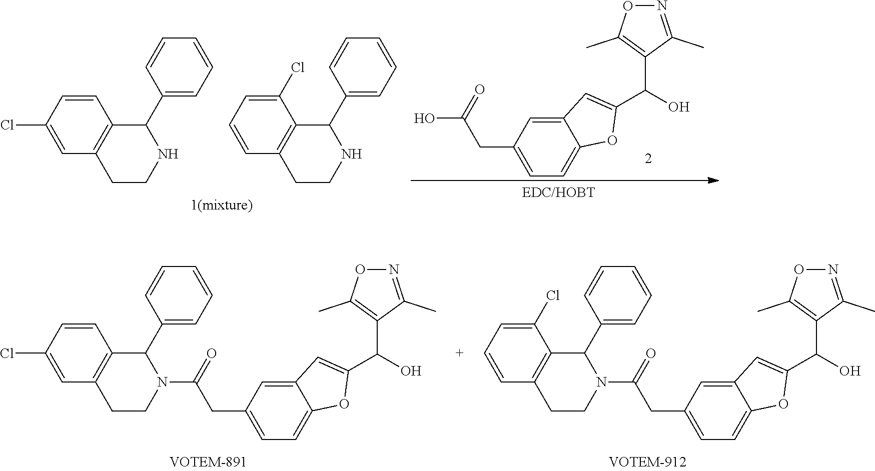

These compound were synthesized from 6-chloro-1-phenyl-1,2,3,4-tetrahydroisoquinoline and 8-chloro-1-phenyl-1,2,3,4-tetrahydroisoquinoline and 2-(2-((3,5-dimethylisoxazol-4-yl)(hydroxy)methyl)benzofuran-5-yl)acetic acid essentially as described in example 1 (k):

1-(6-chloro-1-phenyl-3,4-dihydroisoquinolin-2(1H)-yl)-2-(2-((3,5-dimethylisoxazol-4-yl)(hydroxy)methyl)benzofuran-5-yl)ethanone: 45 mg; LC-MS (020): 527.2 [M+H]⁺; Rt: 1.94 min. ¹H NMR (MeOD, 400 MHz): δ 7.42-7.37 (m, 2H), 7.29-7.27 (m, 3H), 7.20-7.06 (m, 6H), 6.87 (s, 1H), 6.68 (s, 1H), 5.87 (s, 1H), 3.97-3.95 (m, 3H), 3.33-3.32 (m, 1H), 2.71-2.68 (m, 2H), 2.38 (s, 3H), 2.21 (s, 3H).

1-(8-chloro-1-phenyl-3,4-dihydroisoquinolin-2(1H)-yl)-2-(2-((3,5-dimethylisoxazol-4-yl)(hydroxy)methyl)benzofuran-5-yl)ethanone: 25 mg; LC-MS (020): 527.2 [M+H]⁺; Rt: 1.90 min. ¹H NMR (MeOD, 400 MHz): δ 7.45-7.37 (m, 2H), 7.31-7.25 (m, 5H), 7.20-7.07 (m, 5H), 6.68 (s, 1H), 5.87 (s, 1H), 4.02-3.92 (m, 3H), 3.33-3.29 (m, 1H), 2.73-2.68 (m, 2H), 2.38 (s, 3H), 2.21 (s, 3H).

### Example 61

### 2-(2-((3,5-dimethylisoxazol-4-yl)(hydroxy)methyl)benzofuran-5-yl)-1-(6-methoxy-1-phenyl-3,4-dihydroisoquinolin-2(1H)-yl)ethanone

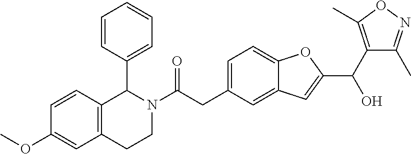

### a) N-benzylidene-2-(3-methoxyphenyl)ethanamine

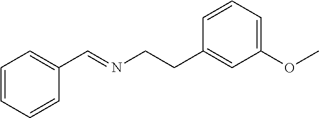 [SMILES: COc1cccc(CC/N=C/c2ccccc2)c1]

This compound was synthesized from 2-(3-methoxyphenyl)ethanamine and benzaldehyde essentially as described in example 58 and 59 (a) (8 g, crude).

### b) 6-methoxy-1-phenyl-1,2,3,4-tetrahydroisoquinoline

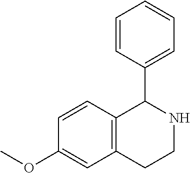 [SMILES: COc1ccc2c(c1)CCNC2c1ccccc1]

A solution of N-benzylidene-2-(3-methoxyphenyl)ethanamine (2 g, 8.4 mmol) in TFA (8 ml) was irradiated at 150 W in a microwave oven at 90° C. for 10 min. The mixture was then poured into 10 ml ice-water, basified to pH˜8-9 with 2N NaOH, and extracted with CH₂Cl₂ (50 ml). The combined extracts were washed with 20 ml brine, dried over sodium sulfate, and concentrated under reduced pressure. The resulting residue was recrystallized from MeOH and petroleum ether to afford 6-methoxy-1-phenyl-1,2,3,4-tetrahydroisoquinoline (0.68 g, yield: 34%) as white solid. LCMSA (044): 240 [M+H]⁺; Rt: 1.75 min.

### c) 2-(2-((3,5-dimethylisoxazol-4-yl)(hydroxy)methyl)benzofuran-5-yl)-1-(6-methoxy-1-phenyl-3,4-dihydroisoquinolin-2(1H)-yl)ethanone

This compound was synthesized from 6-methoxy-1-phenyl-1,2,3,4-tetrahydroisoquinoline and 2-(2-((3,5-dimethylisoxazol-4-yl)(hydroxy)methyl)benzofuran-5-yl)acetic acid essentially as described in example 1 (k) (yield: 27.8%). LC-MS (020): 523.2 [M+H]⁺; Rt: 1.86 min. ¹H NMR (MeOD, 400 MHz): δ 7.43-7.37 (m, 2H), 7.26-7.23 (m, 3H), 7.18-7.16 (m, 3H), 6.99-6.97 (m, 1H), 6.81-6.67 (m, 4H), 5.87 (s, 1H), 3.96-3.95 (m, 3H), 3.78 (s, 3H), 3.37-3.32 (m, 1H), 2.66-2.62 (m, 2H), 2.38 (s, 3H), 2.21 (s, 3H).

### Example 62

### 2-(2-((3,5-dimethylisoxazol-4-yl)(hydroxy)methyl)benzofuran-5-yl)-N-((2,4-dimethylphenyl)(4-fluorophenyl)methyl)acetamide

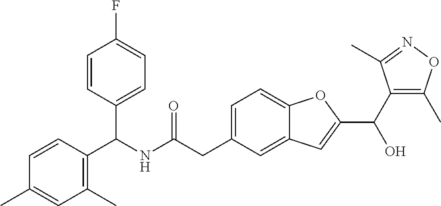

### a) (2,4-dimethylphenyl)(4-fluorophenyl)methanamine

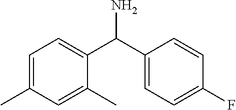 [SMILES: Cc1ccc(C(N)c2ccc(F)cc2)c(C)c1]

4-Fluoro phenyl magnesium bromide (0.8 M in THF, 14.3 mL, 11.45 mmol) was added very slowly to a solution of 2,4-dimethylbenzonitrile (1.0 g, 7.63 mmol) in anhydrous THF (15 mL) at 0° C. After 10 min the reaction mixture was warmed to rt, and stirred for 1 h. The reaction mixture was then cooled to 0° C., and MeOH (5 mL) was added very slowly, followed by sodium borohydride (0.433 g, 11.45 mmol). The resulted mixture was refluxed for 4 h and then stirred at rt overnight. Crushed ice was then added to the reaction mixture. The reaction mixture was extracted with ethyl acetate (40 mL), and the combined extracts were washed with brine (15 mL), dried over Na₂SO₄ and concentrated under reduced pressure to obtained a crude product which was purified using silica gel chromatography using 20% EtOAc:Hexanes to yield (2,4-dimethylphenyl)(4-fluorophenyl)methanamine (0.500 g, 28.73%) as a light yellow semi solid. ¹H NMR (400 MHz, DMSO) δ 6.90-7.37 (m, 7H), 5.19 (s, 1H), 2.22 (s, 3H), 2.13 (s, 5H).

### b) 2-(2-((3,5-dimethylisoxazol-4-yl)(hydroxy)methyl)benzofuran-5-yl)-N-((2,4-dimethylphenyl)(4-fluorophenyl)methyl)acetamide

This compound was synthesized from (2,4-dimethylphenyl)(4-fluorophenyl)methanamine and 2-(2-((3,5-dimethylisoxazol-4-yl)(hydroxy)methyl)benzofuran-5-yl)acetic acid essentially as described in example 1 (k) (50 mg, yield: 36.7%) as white solid. LC-MS (20), MS: 513.70 [M+H]⁺; Rt=1.885 min. ¹H NMR (MeOD, 400 MHz): δ 7.51 (s, 1H), 7.39 (d, J=8.4 Hz, 1H), 7.23 (d, J=8.4 Hz, 1H), 7.16 (t, J=5.6 Hz, 2H), 7.02 (t, J=8.8 Hz, 3H), 6.95 (s, 2H), 6.71 (s, 1H), 6.29 (s, 1H), 5.89 (s, 1H), 3.67 (s, 2H), 2.39 (s, 3H), 2.28 (s, 3H), 2.22 (s, 3H), 2.17 (s, 3H).

### Example 63

### 1-(1,8-dimethyl-5-phenyl-2,3-dihydro-1H-benzo[e][1,4]diazepin-4(5H)-yl)-2-(2-((3,5-dimethylisoxazol-4-yl)(hydroxyl)methyl)benzofuran-5-yl)ethanone

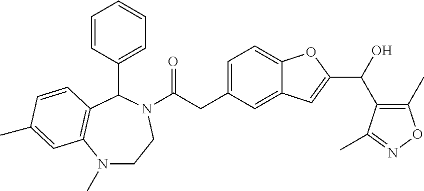

### a) (2-fluoro-4-methylphenyl)(phenyl)methanol

To a stirred solution of 2-fluoro-4-methylbenzaldehyde (1.0 g, 7.24 mmol) in THF (50 mL) was added dropwise a solution of phenylmagnesium bromide (1M in THF, 7.6 mL) at −78° C. under nitrogen. The cooling bath was removed and the reaction mixture was allowed to warm to 0° C. After 2 h, the reaction mixture was diluted with water (60 mL) and diethyl ether (80 mL). The layers were separated and the aqueous layer was back extracted with diethyl ether (50 mL). After separating the layers, the diethyl ether extracts were dried over Na₂SO₄ and concentrated under reduced pressure. The resulting oil (1.56 g, 99%) was used directly in the next step without further purification. LCMS-AMF: 199 (—OH frag.)[no M+H ion]⁺; Rₜ: 5.373 min.

### b) (2-fluoro-4-methylphenyl)(phenyl)methanone

To a stirred solution of (2-fluoro-4-methylphenyl)(phenyl)methanol (1.56 g, 7.24 mmol) in CH₂Cl₂ (50 mL) was added Dess-Martin periodinane (3 g, 7.24 mmol) at rt. After 90 min of stirring at rt, the reaction mixture was quenched with saturated aqueous sodium bicarbonate solution (20 mL). The layers were separated and the aqueous layer was extracted with CH₂Cl₂ (40 mL). The CH₂Cl₂ extracts were combined, washed with brine (20 mL), dried over Na₂SO₄, and concentrated under reduced pressure. The resulting oil was purified by on a silica gel column (1:1 hexanes/EtOAc) to obtain the title compound (1.13 g, 73%) as a white crystalline solid. LCMS-AMF: 215 [M+H]⁺; Rₜ: 5.919 min. ¹H NMR (400 MHz, CDCl₃) δ ppm 7.83 (d, J=8.0 Hz, 2H), 7.65 (m, 1H), 7.45 (m, 3H), 7.07 (d, J=8.0 Hz, 1H), 6.98 (d, J=12.0 Hz, 1H), 2.44 (s, 3H).

### c) 1,8-dimethyl-5-phenyl-2,3-dihydro-1H-benzo[e][1,4]diazepine

To a stirred solution of (2-fluoro-4-methylphenyl)(phenyl)methanone (250 mg, 1.17 mmol) in EtOH (2.5 mL) was added N-methylethylenediamine (340 mg, 4.67 mmol) at rt. The reaction solution was irradiated for 20 min in the microwave reactor at 180° C. The reaction mixture was concentrated under reduced pressure. The resulting oil was diluted with EtOAc (40 mL) and water (20 mL). After the layers were separated, the aqueous layer was extracted twice with EtOAc (40 mL). The EtOAc extracts were combined, washed with brine (40 mL), dried over Na₂SO₄, and concentrated under reduced pressure. The resulting oil was purified by on a silica gel column (1:1 hexanes/EtOAc) to obtain the title compound (79 mg, 27%) as a yellow solid. LCMS-AMF: 251 [M+H]⁺; Rₜ: 6.051 min. ¹H NMR (400 MHz, CDCl₃) δ ppm 7.56 (m, 2H), 7.35 (m, 3H), 6.91 (d, J=8.0 Hz, 1H), 6.76 (m, 2H), 3.74 (dd, J=8.0, 4.0 Hz, 2H), 3.61 (dd, J=8.0, 4.0 Hz, 2H), 2.79 (s, 3H), 2.39 (s, 3H).

### d) 1,8-dimethyl-5-phenyl-2,3,4,5-tetrahydro-1H-benzo[e][1,4]diazepine

To a stirred solution of 1,8-dimethyl-5-phenyl-2,3-dihydro-1H-benzo[e][1,4]diazepine (70 mg, 0.28 mmol) in MeOH (1 mL) and AcOH (17 mg, 0.28 mmol) was added sodium borohydride (11 mg, 0.28 mmol) portionwise at rt. The addition of sodium borohydride to the reaction mixture was continued until the starting material was observed to be consumed by LCMS. Once the reaction was determined to be complete by LCMS, the reaction mixture was concentrated under reduced pressure. The resulting residue was stirred in saturated aqueous sodium bicarbonate solution (10 mL) for 10 min. The mixture was diluted with EtOAc (30 mL) and water (20 mL) and the layers were later separated. The aqueous layer was extracted with EtOAc (30 mL). The EtOAc extracts were combined, dried over Na₂SO₄, and concentrated under reduced pressure. The resulting oil was purified by on a silica gel column (5% MeOH in CH₂Cl₂) to obtain the title compound (29 mg, 41%) as a yellow solid. LCMS-AMF: 253 [M+H]⁺; Rₜ: 6.202 min. ¹H NMR (400 MHz, CDCl₃) δ ppm 7.34 (m, 3H), 7.28 (m, 2H), 6.80 (s, 1H), 6.59 (d, J=8.0 Hz, 1H), 6.48 (d, J=8.0 Hz, 1H), 5.20 (s, 1H), 3.16 (m, 3H), 2.93 (s, 3H), 2.83 (m, 1H), 2.31 (s, 3H).

### e) 1-(1,8-dimethyl-5-phenyl-2,3-dihydro-1H-benzo[e][1,4]diazepin-4(5H)-yl)-2-(2-((3,5-dimethylisoxazol-4-yl)(hydroxyl)methyl)benzofuran-5-yl)ethanone

To a stirred suspension of 2-(2-((3,5-dimethylisoxazol-4-yl)(hydroxyl)methyl)benzofuran-5-yl)acetic acid (20 mg, 0.07 mmol) and HATU (28 mg, 0.07 mmol) in CH₂Cl₂ (0.5 mL) was added DIPEA (15 mg, 0.1 mmol). The resulting mixture was allowed to stir at rt for 20 min and then a solution of 1,8-dimethyl-5-phenyl-2,3,4,5-tetrahydro-1H-benzo[e][1,4]diazepine (17 mg, 0.07 mmol) in CH₂Cl₂ (0.5 mL) was added to the solution. The resulting mixture was stirred at rt for 13 h. CH₂Cl₂ (20 mL) was added to the mixture and the mixture was washed with 50% aqueous sodium bicarbonate solution (10 mL). After separating the layers, the aqueous layer was back extracted with CH₂Cl₂ (20 mL). The CH₂Cl₂ extracts were combined and concentrated under reduced pressure. The concentrated residue was purified by preparative HPLC to obtain the title compound (17 mg, 50%) as a white solid. LCMS-AMF: 536 [M+H]⁺; Rₜ: 6.328 min. ¹H NMR (400 MHz, CDCl₃) δ ppm 7.38-6.95 (m, 10H), 6.63 (m, 2H), 6.49 (s, 1H), 5.82 (s, 1H), 3.83 (s, 2H), 3.73 (m, 1H), 3.27 (m, 2H), 2.94 (m, 1H), 2.77 (s, 3H), 2.35 (s, 3H), 2.34 (s, 3H), 2.17 (s, 3H).

### Biological Data

As stated above, the compounds according to Formula (I) are RORγ modulators, and are useful in the treatment of diseases mediated by RORγ. The biological activities of the compounds according to Formula (I) can be determined using any suitable assay for determining the activity of a candidate compound as a RORγ modulator, as well as tissue and in vivo models.

### Dual Fluorescence Energy Transfer (FRET) Assay

This assay is based on the knowledge that nuclear receptors interact with cofactors (transcription factors) in a ligand dependent manner RORγ is a typical nuclear receptor in that it has an AF2 domain in the ligand binding domain (LBD) which interacts with co-activators. The sites of interaction have been mapped to the LXXLL motifs in the co-activator SRC1(2) sequences. Short peptide sequences containing the LXXLL motif mimic the behavior of full-length co-activator.

The assay measures ligand-mediated interaction of the co-activator peptide with the purified bacterial-expressed RORγ ligand binding domain (RORγ-LBD) to indirectly assess ligand binding. RORγ has a basal level of interaction with the co-activator SRC1(2) in the absence of ligand, thus it is possible to find ligands that inhibit or enhance the RORγ/SRC1(2) interaction.

### Materials

### Generation of RORγ-LBD Bacterial Expression Plasmid

Human RORγ Ligand Binding Domain (RORγ-LBD) was expressed in E. coli strain BL21(DE3) as an amino-terminal polyhistidine tagged fusion protein. DNA encoding this recombinant protein was sub-cloned into a modified pET21a expression vector (Novagen). A modified polyhistidine tag (MKKHHHHHHLVPRGS) (SEQ ID No: 1) was fused in frame to residues 263-518 of the human RORγ sequence.

### Protein Purification

Approximately 50 g E. coli cell pellet was resuspended in 300 mL of lysis buffer (30 mM imidazole pH 7.0 and 150 mM NaCl). Cells were lysed by sonication and cell debris was removed by centrifugation for 30 min at 20,000 g at 4° C. The cleared supernatant was filtered through a 0.45 μM cellulose acetate membrane filter. The clarified lysate was loaded onto a column (XK-26) packed with ProBond Nickel Chelating resin (InVitrogen), pre-equilibrated with 30 mM imidazole pH 7.0 and 150 mM NaCl. After washing to baseline absorbance with the equilibration buffer, the column was developed with a gradient from 30 to 500 mM imidazole pH 7.0. Column fractions containing the RORγ-LBD protein were pooled and concentrated to a volume of 5 mL. The concentrated protein was loaded onto a Superdex 200 column pre-equilibrated with 20 mM Tris-Cl pH 7.2 and 200 mM NaCl. The fractions containing the desired RORγ-LBD protein were pooled together.

### Protein Biotinylation

Purified RORγ-LBD was buffer exchanged by exhaustive dialysis [3 changes of at least 20 volumes (>8000×)] against PBS [100 mM NaPhosphate, pH 8 and 150 mM NaCl]. The concentration of RORγ-LBD was approximately 30 μM in PBS. Five-fold molar excess of NHS-LC-Biotin (Pierce) was added in a minimal volume of PBS. This solution was incubated with occasional gentle mixing for 60 min at rt. The modified RORγ-LBD was dialyzed against 2 buffer changes—TBS pH 8.0 containing 5 mM DTT, 2 mM EDTA and 2% sucrose—each at least 20 times of the volume. The modified protein was distributed into aliquots, frozen on dry ice and stored at −80° C. The biotinylated RORγ-LBD was subjected to mass spectrometric analysis to reveal the extent of modification by the biotinylation reagent. In general, approximately 95% of the protein had at least a single site of biotinylation and the overall extent of biotinylation followed a normal distribution of multiple sites ranged from one to five.

A biotinylated peptide corresponding to amino acid 676 to 700 (CPSSHSSLTERHKILHRLLQEGSPS) (SEQ ID No: 2) of the co-activator steroid receptor coactivator SRC1(2) was generated using similar method.

### Assay

Preparation of Europium labeled SRC1(2) peptide: biotinylated SRC1(2) solution was prepared by adding an appropriate amount of biotinylated SRC1(2) from the 100 μM stock solution to a buffer containing 10 mM of freshly added DTT from solid to give a final concentration of 40 nM. An appropriate amount of Europium labeled Streptavidin was then added to the biotinylated SRC1(2) solution in a tube to give a final concentration of 10 nM. The tube was inverted gently and incubated for 15 min at rt. Twenty-fold excess biotin from the 10 mM stock solution was added and the tube was inverted gently and incubated for 10 min at rt.

Preparation of APC labeled RORγ-LBD: biotinylated RORγ-LBD solution was prepared by adding an appropriate amount of biotinylated RORγ-LBD from the stock solution to a buffer containing 10 mM of freshly added DTT from solid to give a final concentration of 40 nM. An appropriate amount of APC labeled Streptavidin was then added to the biotinylated RORγ-LBD solution in a tube to give a final concentration of 20 nM. The tube was inverted gently and incubated for 15 min at rt. Twenty-fold excess biotin from the 10 mM stock solution was then added and the tube was inverted gently and incubated for 10 min at rt.

Equal volumes of the above-described Europium labeled SRC1(2) peptide and the APC labeled RORγ-LBD were gently mixed together to give 20 nM RORγ-LBD, 10 nM APC-Strepavidin, 20 nM SRC1(2) and 5 nM Europium-Streptavidin. The reaction mixtures were incubated for 5 min. Using a Thermo Combi Multidrop 384 stacker unit, 25 μL of the reaction mixtures per well was added to the 384-well assay plates containing 1 μL of test compound per well in 100% DMSO. The plates were incubated for 1 hour and then read on ViewLux in Lance mode for EU/APC.

### Results

All exemplified compounds (Examples 1-63) were tested in the dual FRET assay described above and were found to have a pIC₅₀ between 5 and 9.

1. A compound according to Formula (I) or a pharmaceutically acceptable salt thereof: 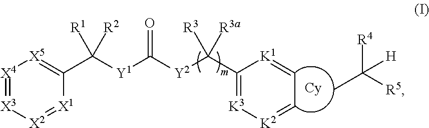
wherein:
m is 0, 1, or 2;
X¹, X², X³, X⁴, and X⁵ are each independently selected from N, N⁺—O⁻, CH, and CR⁶, wherein 0-3 of X¹, X², X³, X⁴, and X⁵ are N or N⁺—O⁻ and 0-3 of X¹, X², X³, X⁴, and X⁵ are CR⁶;
one of Y¹ and Y² is O or NR⁸ and the other is a bond;
or X¹ is CR⁶, Y¹ is NR⁸, Y² is a bond, and R⁶ and R⁸ taken together with the atoms to which they are attached form a five to seven membered ring, optionally containing an additional heteroatom selected from oxygen, nitrogen, and sulfur, which ring is optionally substituted by (C₁-C₄)alkyl;
K¹, K², and K³ are each independently selected from N, CH, and CR⁶, wherein 0-2 of K¹, K², and K³ are N and 0-2 of K¹, K², and K³ are CR⁶;
R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;
R² is hydrogen, (C₁-C₆)alkyl, or (C₁-C₆)haloalkyl;
or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;
R³ and R^{3a} are each independently hydrogen, hydroxyl, (C₁-C₆)alkyl, (C₁-C₆)haloalkyl, halogen, (C₁-C₆)alkoxy, amino, (C₁-C₄)alkylamino, or ((C₁-C₄)alkyl)((C₁-C₄)alkyl)amino;
R⁴ is hydroxyl or amino;
R⁵ is phenyl or 5- or 6-membered heteroaryl, wherein said phenyl or heteroaryl is optionally substituted one, two, or three times, independently, by (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)CO₂R⁷, —((C₀-C₃)alkyl)CONR⁷R⁸, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;
each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;
R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;
R⁸ is hydrogen, (C₁-C₆)alkyl, or (C₁-C₆)haloalkyl;
or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;
R⁹ is (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl; and
Cy taken together with the two carbon atoms of the phenyl or heteroaryl group to which it is fused comprises a five or six membered ring, optionally containing one, two, or three heteroatoms independently selected from oxygen, nitrogen, and sulfur, which ring is optionally substituted one or two times, independently, by R⁹;
provided that the compound of Formula (I) is not: 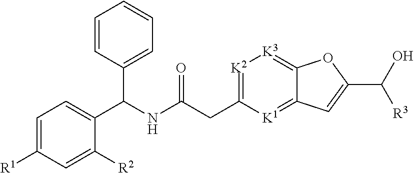
wherein:
K¹, K², and K³ are each independently selected from N and CH, wherein 0-2 of K¹, K², and K³ are N;
R¹ is F, Cl, —CH₃, or —OCH₃;
R² is —CH₃, —CN, —N(CH₃)₂, or —OCH₃; and
R³ is phenyl or 5- or 6-membered heteroaryl, wherein said phenyl or heteroaryl is optionally substituted one, two or three times, independently, by (C₁-C₄)alkyl, (C₁-C₄)haloalkyl, (C₃-C₆)cycloalkyl, halogen, cyano, hydroxyl, hydroxy(C₁-C₆)alkyl, (C₁-C₄)alkoxy, —((C₀-C₃)alkyl)CO₂(C₁-C₄)alkyl, —((C₀-C₃)alkyl)CONH₂, —((C₀-C₃)alkyl)CONH(C₁-C₄)alkyl, —((C₀-C₃)alkyl)CON((C₁-C₄)alkyl)((C₁-C₄)alkyl), or (C₁-C₄)alkoxy(C₁-C₆)alkyl.

2. The compound or pharmaceutically acceptable salt according to claim 1, wherein m is 1.

3. The compound or pharmaceutically acceptable salt according to claim 1, wherein X¹, X², X³, X⁴, and X⁵ are each independently selected from CH and CR⁶, wherein 0-3 of X¹, X², X³, X⁴, and X⁵ are CR⁶.

4. The compound or pharmaceutically acceptable salt according to claim 1, wherein X¹ is a carbon atom substituted by halogen, (C₁-C₄)alkyl, (C₁-C₄)haloalkyl, cyano, (C₁-C₄)alkoxy, or ((C₁-C₄)alkyl)((C₁-C₄)alkyl)amino, and X², X³, X⁴, and X⁵ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino, wherein 2-4 of X², X³, X⁴, and X⁵ are a carbon atom substituted by hydrogen.

5. The compound or pharmaceutically acceptable salt according to claim 1, wherein Y¹ is NH or NCH₃ and Y² is a bond.

6. The compound or pharmaceutically acceptable salt according to claim 1, wherein K¹, K², and K³ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino, wherein 1-3 of K¹, K², and K³ are a carbon atom substituted by hydrogen.

7. The compound or pharmaceutically acceptable salt according to claim 1, wherein R¹ is (C₃-C₆)alkyl, (C₃-C₆)cycloalkyl, (C₁-C₆)alkoxy(C₁-C₂)alkyl, phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein said phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl is optionally substituted one or two times, independently, by halogen, (C₁-C₄)alkyl, (C₁-C₄)haloalkyl, cyano (C₁-C₄)alkoxy, or ((C₁-C₄)alkyl)((C₁-C₄)alkyl)amino.

8. The compound or pharmaceutically acceptable salt according to claim 1, wherein R¹ is phenyl or pyridinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino.

9. The compound or pharmaceutically acceptable salt according to claim 1, wherein R² is hydrogen or methyl.

10. The compound or pharmaceutically acceptable salt according to claim 1, wherein R³ and R^{3a} are each independently hydrogen or methyl.

11. The compound or pharmaceutically acceptable salt according to claim 1, wherein R⁴ is hydroxyl.

12. The compound or pharmaceutically acceptable salt according to claim 1, wherein R⁴ is amino.

13. The compound or pharmaceutically acceptable salt according to claim 1, wherein R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino.

14. The compound according to claim 1 having Formula (Ia): 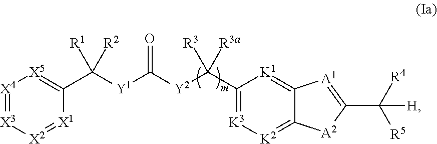
wherein:
m is 1;
X¹, X², X³, X⁴, and X⁵ are each independently selected from N, N⁺—O⁻, CH, and CR⁶, wherein 0-2 of X¹, X², X³, X⁴, and X⁵ are N or N⁺—O⁻ and 0-3 of X¹, X², X³, X⁴, and X⁵ are CR⁶;
Y¹ is NH or NCH₃ and Y² is a bond;
K¹, K², and K³ are each independently selected from N, CH, and CR⁶, wherein 0-1 of K¹, K², and K³ are N and 0-1 of K¹, K², and K³ are CR⁶;
A¹ is N, CH, or CR⁹;
A² is O, S, NH, NR⁷, NC(O)R⁷, NCO₂R⁷, or NC(O)NR⁷R⁸;
R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;
R² is hydrogen, (C₁-C₆)alkyl, or (C₁-C₆)haloalkyl;
or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;
R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;
R⁴ is hydroxyl or amino;
R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;
each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;
R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;
R⁸ is hydrogen, (C₁-C₆)alkyl, or (C₁-C₆)haloalkyl;
or R⁷ and R⁸ taken together with the nitrogen atom to which they are attached form a four to eight membered ring, optionally containing an additional heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂H, —CO₂(C₁-C₄)alkyl, CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷; and
R⁹ is (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;
or a pharmaceutically acceptable salt thereof.

15. The compound or pharmaceutically acceptable salt according to claim 14, wherein:
X¹ is a carbon atom substituted by halogen, (C₁-C₄)alkyl, (C₁-C₄)haloalkyl, cyano, (C₁-C₄)alkoxy, or ((C₁-C₄)alkyl)((C₁-C₄)alkyl)amino, and X², X³, X⁴, and X⁵ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino, wherein 2-4 of X², X³, X⁴, and X⁵ are a carbon atom substituted by hydrogen;
Y¹ is NH and Y² is a bond;
K¹, K², and K³ are each independently CH;
A¹ is N or CH;
A² is O, S, NH, or N((C₁-C₄)alkyl);
R¹ is phenyl optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;
R² is hydrogen;
R³ and R^{3a} are each independently hydrogen or methyl;
R⁴ is hydroxyl; and
R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, each of which is optionally substituted one or two times, independently, by (C₁-C₄)alkyl.

16. The compound according to claim 1 having Formula (Ib): 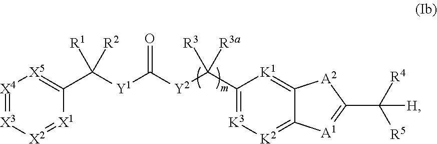
wherein:
m is 1;
X¹, X², X³, X⁴, and X⁵ are each independently selected from N, N⁺—O⁻, CH, and CR⁶, wherein 0-2 of X¹, X², X³, X⁴, and X⁵ are N or N⁺—O⁻ and 0-3 of X¹, X², X³, X⁴, and X⁵ are CR⁶;
Y¹ is NH or NCH₃ and Y² is a bond;
K¹, K², and K³ are each independently selected from N, CH, and CR⁶, wherein 0-1 of K¹, K², and K³ are N and 0-1 of K¹, K², and K³ are CR⁶;
A¹ is N, CH, or CR⁹;
A² is O, S, NH, NR⁷, NC(O)R⁷, NCO₂R⁷, or NC(O)NR⁷R⁸;
R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;
R² is hydrogen, (C₁-C₆)alkyl, or (C₁-C₆)haloalkyl;
or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;
R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;
R⁴ is hydroxyl or amino;
R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;
each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;
R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;
R⁸ is hydrogen, (C₁-C₆)alkyl, or (C₁-C₆)haloalkyl;
or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino; and
R⁹ is (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;
or a pharmaceutically acceptable salt thereof.

17. The compound or pharmaceutically acceptable salt according to claim 16, wherein:
X¹ is a carbon atom substituted by halogen, (C₁-C₄)alkyl, (C₁-C₄)haloalkyl, cyano, (C₁-C₄)alkoxy, or ((C₁-C₄)alkyl)((C₁-C₄)alkyl)amino, and X², X³, X⁴, and X⁵ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino, wherein 2-4 of X², X³, X⁴, and X⁵ are a carbon atom substituted by hydrogen;
Y¹ is NH and Y² is a bond;
K¹, K², and K³ are each independently CH;
A¹ is N or CH;
A² is O, S, NH, or N((C₁-C₄)alkyl);
R¹ is phenyl optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;
R² is hydrogen;
R³ and R^{3a} are each independently hydrogen or methyl;
R⁴ is hydroxyl; and
R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, each of which is optionally substituted one or two times, independently, by (C₁-C₄)alkyl.

18. The compound according to claim 1 having Formula (Ic): 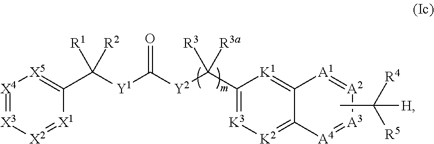
wherein:
m is 1;
X¹, X², X³, X⁴, and X⁵ are each independently selected from N, N⁺—O⁻, CH, and CR⁶, wherein 0-2 of X¹, X², X³, X⁴, and X⁵ are N or N⁺—O⁻ and 0-3 of X¹, X², X³, X⁴, and X⁵ are CR⁶;
Y¹ is NH or NCH₃ and Y² is a bond;
K¹, K², and K³ are each independently selected from N, CH, and CR⁶, wherein 0-1 of K¹, K², and K³ are N and 0-1 of K¹, K², and K³ are CR⁶;
A¹, A², A³, and A⁴ are each independently selected from N, C, CH, and CR⁹, wherein 0-2 of A¹, A², A³, and A⁴ are N, 0-1 of A¹, A², A³, and A⁴ are CR⁹, and 1 of A¹, A², A³, and A⁴ is C to which CHR⁴R⁵ is attached;
R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;
R² is hydrogen, (C₁-C₆)alkyl, or (C₁-C₆)haloalkyl;
or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;
R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;
R⁴ is hydroxyl or amino;
R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or (C₁-C₄)alkyl)((C₁-C₄)alkyl)amino;
each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;
R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;
R⁸ is hydrogen, (C₁-C₆)alkyl, or (C₁-C₆)haloalkyl; and
R⁹ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;
or a pharmaceutically acceptable salt thereof.

19. The compound according to claim 1 having Formula (Id): 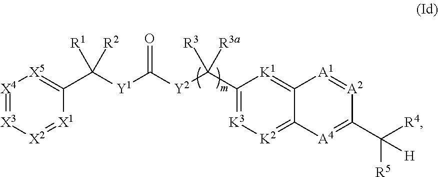
wherein:
m is 1;
X¹, X², X³, X⁴, and X⁵ are each independently selected from N, N⁺—O⁻, CH, and CR⁶, wherein 0-2 of X¹, X², X³, X⁴, and X⁵ are N or N⁺—O⁻ and 0-3 of X¹, X², X³, X⁴, and X⁵ are CR⁶;
Y¹ is NH or NCH₃ and Y² is a bond;
K¹, K², and K³ are each independently selected from N, CH, and CR⁶, wherein 0-1 of K¹, K², and K³ are N and 0-1 of K¹, K², and K³ are CR⁶;
A¹, A², and A⁴ are each independently selected from N, CH, and CR⁹, wherein 0-2 of A¹, A², and A⁴ are N, and 0-1 of A¹, A², and A⁴ are CR⁹;
R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;
R² is hydrogen, (C₁-C₆)alkyl, or (C₁-C₆)haloalkyl;
or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;
R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;
R⁴ is hydroxyl or amino;
R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;
each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl
R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;
R⁸ is hydrogen, (C₁-C₆)alkyl, or (C₁-C₆)haloalkyl; and
R⁹ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)NHCO₂R⁷,
—((C₀-C₃)alkyl)N((C₁-C₄)alkyl)CO₂R⁷, —((C₀-C₃)alkyl)NHC(O)R⁷,
—((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;
or a pharmaceutically acceptable salt thereof.

20-22. (canceled)

23. A pharmaceutical composition comprising the compound, or pharmaceutically acceptable salt thereof, according to claim 1 and a pharmaceutically acceptable excipient.

24-34. (canceled)
